SEQUENCE LISTING (35 sequences)

SEQ ID NO: 1 - DNA, 10600 bp, Artificial Sequence
  TMV infectious clone containing CEL I gene
gtatttttacaacaattaccaacaacaacaaacaacaaacaacattacaattactatttacaattacaatggcatacacacagacagctaccacatcagctttgctggacactgtccgaggaaacaactccttggtcaatgatctagcaaagcgtcgtctttacgacacagcggttgaagagtttaacgctcgtgaccgcaggcccaaggtgaacttttcaaaagtaataagcgaggagcagacgcttattgctacccgggcgtatccagaattccaaattacattttataacacgcaaaatgccgtgcattcgcttgcaggtggattgcgatctttagaactggaatatctgatgatgcaaattccctacggatcattgacttatgacataggcgggaattttgcatcgcatctgttcaagggacgagcatatgtacactgctgcatgcccaacctggacgttcgagacatcatgcggcacgaaggccagaaagacagtattgaactatacctttctaggctagagagaggggggaaaacagtccccaacttccaaaaggaagcatttgacagatacgcagaaattcctgaagacgctgtctgtcacaatactttccagacaatgcgacatcagccgatgcagcaatcaggcagagtgtatgccattgcgctacacagcatatatgacataccagccgatgagttcggggcggcactcttgaggaaaaatgtccatacgtgctatgccgctttccacttctctgagaacctgcttcttgaagattcatacgtcaatttggacgaaatcaacgcgtgtttttcgcgcgatggagacaagttgaccttttcttttgcatcagagagtactcttaattattgtcatagttattctaatattcttaagtatgtgtgcaaaacttacttcccggcctctaatagagaggtttacatgaaggagtttttagtcaccagagttaatacctggttttgtaagttttctagaatagatacttttcttttgtacaaaggtgtggcccataaaagtgtagatagtgagcagttttatactgcaatggaagacgcatggcattacaaaaagactcttgcaatgtgcaacagcgagagaatcctccttgaggattcatcatcagtcaattactggtttcccaaaatgagggatatggtcatcgtaccattattcgacatttctttggagactagtaagaggacgcgcaaggaagtcttagtgtccaaggatttcgtgtttacagtgcttaaccacattcgaacataccaggcgaaagctcttacatacgcaaatgttttgtcctttgtcgaatcgattcgatcgagggtaatcattaacggtgtgacagcgaggtccgaatgggatgtggacaaatctttgttacaatccttgtccatgacgttttacctgcatactaagcttgccgttctaaaggatgacttactgattagcaagtttagtctcggttcgaaaacggtgtgccagcatgtgtgggatgagatttcgctggcgtttgggaacgcatttccctccgtgaaagagaggctcttgaacaggaaacttatcagagtggcaggcgacgcattagagatcagggtgcctgatctatatgtgaccttccacgacagattagtgactgagtacaaggcctctgtggacatgcctgcgcttgacattaggaagaagatggaagaaacggaagtgatgtacaatgcactttcagagttatcggtgttaagggagtctgacaaattcgatgttgatgttttttcccagatgtgccaatctttggaagttgacccaatgacggcagcgaaggttatagtcgcggtcatgagcaatgagagcggtctgactctcacatttgaacgacctactgaggcgaatgttgcgctagctttacaggatcaagagaaggcttcagaaggtgctttggtagttacctcaagagaagttgaagaaccgtccatgaagggttcgatggccagaggagagttacaattagctggtcttgctggagatcatccggagtcgtcctattctaagaacgaggagatagagtctttagagcagtttcatatggcaacggcagattcgttaattcgtaagcagatgagctcgattgtgtacacgggtccgattaaagttcagcaaatgaaaaactttatcgatagcctggtagcatcactatctgctgcggtgtcgaatctcgtcaagatcctcaaagatacagctgctattgaccttgaaacccgtcaaaagtttggagtcttggatgttgcatctaggaagtggttaatcaaaccaacggccaagagtcatgcatggggtgttgttgaaacccacgcgaggaagtatcatgtggcgcttttggaatatgatgagcagggtgtggtgacatgcgatgattggagaagagtagctgtcagctctgagtctgttgtttattccgacatggcgaaactcagaactctgcgcagactgcttcgaaacggagaaccgcatgtcagtagcgcaaaggttgttcttgtggacggagttccgggctgtgggaaaaccaaagaaattctttccagggttaattttgatgaagatctaattttagtacctgggaagcaagccgcggaaatgatcagaagacgtgcgaattcctcagggattattgtggccacgaaggacaacgttaaaaccgttgattctttcatgatgaattttgggaaaagcacacgctgtcagttcaagaggttattcattgatgaagggttgatgttgcatactggttgtgttaattttcttgtggcgatgtcattgtgcgaaattgcatatgtttacggagacacacagcagattccatacatcaatagagtttcaggattcccgtaccccgcccattttgccaaattggaagttgacgaggtggagacacgcagaactactctccgttgtccagccgatgtcacacattatctgaacaggagatatgagggctttgtcatgagcacttcttcggttaaaaagtctgtttcgcaggagatggtcggcggagccgccgtgatcaatccgatctcaaaacccttgcatggcaagatcctgacttttacccaatcggataaagaagctctgctttcaagagggtattcagatgttcacactgtgcatgaagtgcaaggcgagacatactctgatgtttcactagttaggttaacccctacaccagtctccatcattgcaggagacagcccacatgttttggtcgcattgtcaaggcacacctgttcgctcaagtactacactgttgttatggatcctttagttagtatcattagagatctagagaaacttagctcgtacttgttagatatgtataaggtcgatgcaggaacacaatagcaattacagattgactcggtgttcaaaggttccaatctttttgttgcagcgccaaagactggtgatatttctgatatgcagttttactatgataagtgtctcccaggcaacagcaccatgatgaataattttgatgctgttaccatgaggttgactgacatttcattgaatgtcaaagattgcatattggatatgtctaagtctgttgctgcgcctaaggatcaaatcaaaccactaatacctatggtacgaacggcggcagaaatgccacgccagactggactattggaaaatttagtggcgatgattaaaaggaactttaacgcacccgagttgtctggcatcattgatattgaaaatactgcatctttagttgtagataagttttttgatagttatttgcttaaagaaaaaagaaaaccaaataaaaatgtttctttgttcagtagagagtctctcaatagatggttagaaaagcaggaacaggtaacaataggccagctcgcagattttgattttgtagatttgccagcagttgatcagtacagacacatgattaaagcacaacccaagcaaaaattggacacttcaatccaaacggagtacccggctttgcagacgattgtgtaccattcaaaaaagatcaatgcaatatttggcccgttgtttagtgagcttactaggcaattactggacagtgttgattcgagcagatttttgtttttcacaagaaagacaccagcgcagattgaggatttcttcggagatctcgacagtcatgtgccgatggatgtcttggagctggatatatcaaaatacgacaaatctcagaatgaattccactgtgcagtagaatacgagatctggcgaagattgggttttgaagacttcttgggagaagtttggaaacaagggcatagaaagaccaccctcaaggattataccgcaggtataaaaacttgcatctggtatcaaagaaagagcggggacgtcacgacgttcattggaaacactgtgatcattgctgcatgtttggcctcgatgcttccgatggagaaaataatcaaaggagccttttgcggtgacgatagtctgctgtactttccaaagggttgtgagtttccggatgtgcaacactccgcgaatcttatgtggaattttgaagcaaaactgtttaaaaaacagtatggatacttttgcggaagatatgtaatacatcacgacagaggatgcattgtgtattacgatcccctaaagttgatctcgaaacttggtgctaaacacatcaaggattgggaacacttggaggagttcagaaggtctctttgtgatgttgctgtttcgttgaacaattgtgcgtattacacacagttggacgacgctgtatgggaggttcataagaccgcccctccaggttcgtttgtttataaaagtctggtgaagtatttgtctgataaagttctttttagaagtttgtttatagatggctctagttgttaaaggaaaagtgaatatcaatgagtttatcgacctgacaaaaatggagaagatcttaccgtcgatgtttacccctgtaaagagtgttatgtgttccaaagttgataaaataatggttcatgagaatgagtcattgtcagaggtgaaccttcttaaaggagttaagcttattgatagtggatacgtctgtttagccggtttggtcgtcacgggcgagtggaacttgcctgacaattgcagaggaggtgtgagcgtgtgtctggtggacaaaaggatggaaagagccgacgaggccactctcggatcttactacacagcagctgcaaagaaaagatttcagttcaaggtcgttcccaattatgctataaccacccaggacgcgatgaaaaacgtctggcaagttttagttaatattagaaatgtgaagatgtcagcgggtttctgtccgctttctctggagtttgtgtcggtgtgtattgtttatagaaataatataaaattaggtttgagagagaagattacaaacgtgagagacggagggcccatggaacttacagaagaagtcgttgatgagttcatggaagatgtccctatgtcgatcaggcttgcaaagtttcgatctcgaaccggaaaaaagagtgatgtccgcaaagggaaaaatagtagtaatgatcggtcagtgccgaacaagaactatagaaatgttaaggattttggaggaatgagttttaaaaagaataatttaatcgatgatgattcggaggctactgtcgccgaatcggattcgttttaaatagatcttacagtatcactactccatctcagttcgtgttcttgtcattaattaaatgacgcgattatattctgtgttctttcttttgttggctcttgtagttgaaccgggtgttagagcctggagcaaagaaggccatgtcatgacatgtcaaattgcgcaggatctgttggagccagaagcagcacatgctgtaaagatgctgttaccggactatgctaatggcaacttatcgtcgctgtgtgtgtggcctgatcaaattcgacactggtacaagtacaggtggactagctctctccatttcatcgatacacctgatcaagcctgttcatttgattaccagagagactgtcatgatccacatggagggaaggacatgtgtgttgctggagccattcaaaatttcacatctcagcttggacatttccgccatggaacatctgatcgtcgatataatatgacagaggctttgttatttttatcccacttcatgggagatattcatcagcctatgcatgttggatttacaagtgatatgggaggaaacagtatagatttgcgctggtttcgccacaaatccaacctgcaccatgtttgggatagagagattattcttacagctgcagcagattaccatggtaaggatatgcactctctcctacaagacatacagaggaactttacagagggtagttggttgcaagatgttgaatcctggaaggaatgtgatgatatctctacttgcgccaataagtatgctaaggagagtataaaactagcctgtaactggggttacaaagatgttgaatctggcgaaactctgtcagataaatacttcaacacaagaatgccaattgtcatgaaacggatagctcagggtggaatccgtttatccatgattttgaaccgagttcttggaagctccgcagatcattctttggcatgacctaggccagtagtttggtttaaacccaactgcgaggggtagtcaagatgcataataaataacggattgtgtccgtaatcacacgtggtgcgtacgataacgcatagtgtttttccctccacttaaatcgaagggttgtgtcttggatcgcgcgggtcaaatgtatatggttcatatacatccgcaggcacgtaataaagcgaggggttcgggtcgaggtcggctgtgaaactcgaaaaggttccggaaaacaaaaaagagatggtaggtaatagtgttaataataagaaaataaataatagtggtaagaaaggtttgaaagttgaggaaattgaggataatgtaagtgatgacgagtctatcgcgtcatcgagtacgttttaatcaatatgccttatacaatcaactctccgagccaatttgtttacttaagttccgcttatgcagatcctgtgcagctgatcaatctgtgtacaaatgcattgggtaaccagtttcaaacgcaacaagctaggacaacagtccaacagcaatttgcggatgcctggaaacctgtgcctagtatgacagtgagatttcctgcatcggatttctatgtgtatagatataattcgacgcttgatccgttgatcacggcgttattaaatagcttcgatactagaaatagaataatagaggttgataatcaacccgcaccgaatactactgaaatcgttaacgcgactcagagggtagacgatgcgactgtagctataagggcttcaatcaataatttggctaatgaactggttcgtggaactggcatgttcaatcaagcaagctttgagactgctagtggacttgtctggaccacaactccggctacttagctattgttgtgagatttcctaaaataaagtcactgaagacttaaaattcagggtggctgataccaaaatcagcagtggttgttcgtccacttaaatataacgattgtcatatctggatccaacagttaaaccatgtgatggtgtatactgtggtatggcgtaaaacaacggaaaagtcgctgaagacttaaaattcagggtggctgataccaaaatcagcagtggttgttcgtccacttaaaaataacgattgtcatatctggatccaacagttaaaccatgtgatggtgtatactgtggtatggcgtaaaacaacggagaggttcgaatcctcccctaaccgcgggtagcggcccaggtacccggatgtgttttccgggctgatgagtccgtgaggacgaaacccggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcaagcttaatacgactcactata

SEQ ID NO: 2 - DNA, 10624 bp, Artificial Sequence
  TMV infectous clone containing CEL I gene fused to a 6HIS encoding sequence
gtatttttacaacaattaccaacaacaacaaacaacaaacaacattacaattactatttacaattacaatggcatacacacagacagctaccacatcagctttgctggacactgtccgaggaaacaactccttggtcaatgatctagcaaagcgtcgtctttacgacacagcggttgaagagtttaacgctcgtgaccgcaggcccaaggtgaacttttcaaaagtaataagcgaggagcagacgcttattgctacccgggcgtatccagaattccaaattacattttataacacgcaaaatgccgtgcattcgcttgcaggtggattgcgatctttagaactggaatatctgatgatgcaaattccctacggatcattgacttatgacataggcgggaattttgcatcgcatctgttcaagggacgagcatatgtacactgctgcatgcccaacctggacgttcgagacatcatgcggcacgaaggccagaaagacagtattgaactatacctttctaggctagagagaggggggaaaacagtccccaacttccaaaaggaagcatttgacagatacgcagaaattcctgaagacgctgtctgtcacaatactttccagacaatgcgacatcagccgatgcagcaatcaggcagagtgtatgccattgcgctacacagcatatatgacataccagccgatgagttcggggcggcactcttgaggaaaaatgtccatacgtgctatgccgctttccacttctctgagaacctgcttcttgaagattcatacgtcaatttggacgaaatcaacgcgtgtttttcgcgcgatggagacaagttgaccttttcttttgcatcagagagtactcttaattattgtcatagttattctaatattcttaagtatgtgtgcaaaacttacttcccggcctctaatagagaggtttacatgaaggagtttttagtcaccagagttaatacctggttttgtaagttttctagaatagatacttttcttttgtacaaaggtgtggcccataaaagtgtagatagtgagcagttttatactgcaatggaagacgcatggcattacaaaaagactcttgcaatgtgcaacagcgagagaatcctccttgaggattcatcatcagtcaattactggtttcccaaaatgagggatatggtcatcgtaccattattcgacatttctttggagactagtaagaggacgcgcaaggaagtcttagtgtccaaggatttcgtgtttacagtgcttaaccacattcgaacataccaggcgaaagctcttacatacgcaaatgttttgtcctttgtcgaatcgattcgatcgagggtaatcattaacggtgtgacagcgaggtccgaatgggatgtggacaaatctttgttacaatccttgtccatgacgttttacctgcatactaagcttgccgttctaaaggatgacttactgattagcaagtttagtctcggttcgaaaacggtgtgccagcatgtgtgggatgagatttcgctggcgtttgggaacgcatttccctccgtgaaagagaggctcttgaacaggaaacttatcagagtggcaggcgacgcattagagatcagggtgcctgatctatatgtgaccttccacgacagattagtgactgagtacaaggcctctgtggacatgcctgcgcttgacattaggaagaagatggaagaaacggaagtgatgtacaatgcactttcagagttatcggtgttaagggagtctgacaaattcgatgttgatgttttttcccagatgtgccaatctttggaagttgacccaatgacggcagcgaaggttatagtcgcggtcatgagcaatgagagcggtctgactctcacatttgaacgacctactgaggcgaatgttgcgctagctttacaggatcaagagaaggcttcagaaggtgctttggtagttacctcaagagaagttgaagaaccgtccatgaagggttcgatggccagaggagagttacaattagctggtcttgctggagatcatccggagtcgtcctattctaagaacgaggagatagagtctttagagcagtttcatatggcaacggcagattcgttaattcgtaagcagatgagctcgattgtgtacacgggtccgattaaagttcagcaaatgaaaaactttatcgatagcctggtagcatcactatctgctgcggtgtcgaatctcgtcaagatcctcaaagatacagctgctattgaccttgaaacccgtcaaaagtttggagtcttggatgttgcatctaggaagtggttaatcaaaccaacggccaagagtcatgcatggggtgttgttgaaacccacgcgaggaagtatcatgtggcgcttttggaatatgatgagcagggtgtggtgacatgcgatgattggagaagagtagctgtcagctctgagtctgttgtttattccgacatggcgaaactcagaactctgcgcagactgcttcgaaacggagaaccgcatgtcagtagcgcaaaggttgttcttgtggacggagttccgggctgtgggaaaaccaaagaaattctttccagggttaattttgatgaagatctaattttagtacctgggaagcaagccgcggaaatgatcagaagacgtgcgaattcctcagggattattgtggccacgaaggacaacgttaaaaccgttgattctttcatgatgaattttgggaaaagcacacgctgtcagttcaagaggttattcattgatgaagggttgatgttgcatactggttgtgttaattttcttgtggcgatgtcattgtgcgaaattgcatatgtttacggagacacacagcagattccatacatcaatagagtttcaggattcccgtaccccgcccattttgccaaattggaagttgacgaggtggagacacgcagaactactctccgttgtccagccgatgtcacacattatctgaacaggagatatgagggctttgtcatgagcacttcttcggttaaaaagtctgtttcgcaggagatggtcggcggagccgccgtgatcaatccgatctcaaaacccttgcatggcaagatcctgacttttacccaatcggataaagaagctctgctttcaagagggtattcagatgttcacactgtgcatgaagtgcaaggcgagacatactctgatgtttcactagttaggttaacccctacaccagtctccatcattgcaggagacagcccacatgttttggtcgcattgtcaaggcacacctgttcgctcaagtactacactgttgttatggatcctttagttagtatcattagagatctagagaaacttagctcgtacttgttagatatgtataaggtcgatgcaggaacacaatagcaattacagattgactcggtgttcaaaggttccaatctttttgttgcagcgccaaagactggtgatatttctgatatgcagttttactatgataagtgtctcccaggcaacagcaccatgatgaataattttgatgctgttaccatgaggttgactgacatttcattgaatgtcaaagattgcatattggatatgtctaagtctgttgctgcgcctaaggatcaaatcaaaccactaatacctatggtacgaacggcggcagaaatgccacgccagactggactattggaaaatttagtggcgatgattaaaaggaactttaacgcacccgagttgtctggcatcattgatattgaaaatactgcatctttagttgtagataagttttttgatagttatttgcttaaagaaaaaagaaaaccaaataaaaatgtttctttgttcagtagagagtctctcaatagatggttagaaaagcaggaacaggtaacaataggccagctcgcagattttgattttgtagatttgccagcagttgatcagtacagacacatgattaaagcacaacccaagcaaaaattggacacttcaatccaaacggagtacccggctttgcagacgattgtgtaccattcaaaaaagatcaatgcaatatttggcccgttgtttagtgagcttactaggcaattactggacagtgttgattcgagcagatttttgtttttcacaagaaagacaccagcgcagattgaggatttcttcggagatctcgacagtcatgtgccgatggatgtcttggagctggatatatcaaaatacgacaaatctcagaatgaattccactgtgcagtagaatacgagatctggcgaagattgggttttgaagacttcttgggagaagtttggaaacaagggcatagaaagaccaccctcaaggattataccgcaggtataaaaacttgcatctggtatcaaagaaagagcggggacgtcacgacgttcattggaaacactgtgatcattgctgcatgtttggcctcgatgcttccgatggagaaaataatcaaaggagccttttgcggtgacgatagtctgctgtactttccaaagggttgtgagtttccggatgtgcaacactccgcgaatcttatgtggaattttgaagcaaaactgtttaaaaaacagtatggatacttttgcggaagatatgtaatacatcacgacagaggatgcattgtgtattacgatcccctaaagttgatctcgaaacttggtgctaaacacatcaaggattgggaacacttggaggagttcagaaggtctctttgtgatgttgctgtttcgttgaacaattgtgcgtattacacacagttggacgacgctgtatgggaggttcataagaccgcccctccaggttcgtttgtttataaaagtctggtgaagtatttgtctgataaagttctttttagaagtttgtttatagatggctctagttgttaaaggaaaagtgaatatcaatgagtttatcgacctgacaaaaatggagaagatcttaccgtcgatgtttacccctgtaaagagtgttatgtgttccaaagttgataaaataatggttcatgagaatgagtcattgtcagaggtgaaccttcttaaaggagttaagcttattgatagtggatacgtctgtttagccggtttggtcgtcacgggcgagtggaacttgcctgacaattgcagaggaggtgtgagcgtgtgtctggtggacaaaaggatggaaagagccgacgaggccactctcggatcttactacacagcagctgcaaagaaaagatttcagttcaaggtcgttcccaattatgctataaccacccaggacgcgatgaaaaacgtctggcaagttttagttaatattagaaatgtgaagatgtcagcgggtttctgtccgctttctctggagtttgtgtcggtgtgtattgtttatagaaataatataaaattaggtttgagagagaagattacaaacgtgagagacggagggcccatggaacttacagaagaagtcgttgatgagttcatggaagatgtccctatgtcgatcaggcttgcaaagtttcgatctcgaaccggaaaaaagagtgatgtccgcaaagggaaaaatagtagtaatgatcggtcagtgccgaacaagaactatagaaatgttaaggattttggaggaatgagttttaaaaagaataatttaatcgatgatgattcggaggctactgtcgccgaatcggattcgttttaaatagatcttacagtatcactactccatctcagttcgtgttcttgtcattaattaaatgacgcgattatattctgtgttctttcttttgttggctcttgtagttgaaccgggtgttagagcctggagcaaagaaggccatgtcatgacatgtcaaattgcgcaggatctgttggagccagaagcagcacatgctgtaaagatgctgttaccggactatgctaatggcaacttatcgtcgctgtgtgtgtggcctgatcaaattcgacactggtacaagtacaggtggactagctctctccatttcatcgatacacctgatcaagcctgttcatttgattaccagagagactgtcatgatccacatggagggaaggacatgtgtgttgctggagccattcaaaatttcacatctcagcttggacatttccgccatggaacatctgatcgtcgatataatatgacagaggctttgttatttttatcccacttcatgggagatattcatcagcctatgcatgttggatttacaagtgatatgggaggaaacagtatagatttgcgctggtttcgccacaaatccaacctgcaccatgtttgggatagagagattattcttacagctgcagcagattaccatggtaaggatatgcactctctcctacaagacatacagaggaactttacagagggtagttggttgcaagatgttgaatcctggaaggaatgtgatgatatctctacttgcgccaataagtatgctaaggagagtataaaactagcctgtaactggggttacaaagatgttgaatctggcgaaactctgtcagataaatacttcaacacaagaatgccaattgtcatgaaacggatagctcagggtggaatccgtttatccatgattttgaaccgagttcttggaagctccgcagatcattctttggcaggaggtcaccatcaccatcaccattgacctaggccagtagtttggtttaaacccaactgcgaggggtagtcaagatgcataataaataacggattgtgtccgtaatcacacgtggtgcgtacgataacgcatagtgtttttccctccacttaaatcgaagggttgtgtcttggatcgcgcgggtcaaatgtatatggttcatatacatccgcaggcacgtaataaagcgaggggttcgggtcgaggtcggctgtgaaactcgaaaaggttccggaaaacaaaaaagagatggtaggtaatagtgttaataataagaaaataaataatagtggtaagaaaggtttgaaagttgaggaaattgaggataatgtaagtgatgacgagtctatcgcgtcatcgagtacgttttaatcaatatgccttatacaatcaactctccgagccaatttgtttacttaagttccgcttatgcagatcctgtgcagctgatcaatctgtgtacaaatgcattgggtaaccagtttcaaacgcaacaagctaggacaacagtccaacagcaatttgcggatgcctggaaacctgtgcctagtatgacagtgagatttcctgcatcggatttctatgtgtatagatataattcgacgcttgatccgttgatcacggcgttattaaatagcttcgatactagaaatagaataatagaggttgataatcaacccgcaccgaatactactgaaatcgttaacgcgactcagagggtagacgatgcgactgtagctataagggcttcaatcaataatttggctaatgaactggttcgtggaactggcatgttcaatcaagcaagctttgagactgctagtggacttgtctggaccacaactccggctacttagctattgttgtgagatttcctaaaataaagtcactgaagacttaaaattcagggtggctgataccaaaatcagcagtggttgttcgtccacttaaatataacgattgtcatatctggatccaacagttaaaccatgtgatggtgtatactgtggtatggcgtaaaacaacggaaaagtcgctgaagacttaaaattcagggtggctgataccaaaatcagcagtggttgttcgtccacttaaaaataacgattgtcatatctggatccaacagttaaaccatgtgatggtgtatactgtggtatggcgtaaaacaacggagaggttcgaatcctcccctaaccgcgggtagcggcccaggtacccggatgtgttttccgggctgatgagtccgtgaggacgaaacccggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcaagcttaatacgactcactata

SEQ ID NO: 3 - DNA, 891 bp, Apium graveolens
atgacgcgattatattctgtgttctttcttttgttggctcttgtagttgaaccgggtgttagagcctggagcaaagaaggccatgtcatgacatgtcaaattgcgcaggatctgttggagccagaagcagcacatgctgtaaagatgctgttaccggactatgctaatggcaacttatcgtcgctgtgtgtgtggcctgatcaaattcgacactggtacaagtacaggtggactagctctctccatttcatcgatacacctgatcaagcctgttcatttgattaccagagagactgtcatgatccacatggagggaaggacatgtgtgttgctggagccattcaaaatttcacatctcagcttggacatttccgccatggaacatctgatcgtcgatataatatgacagaggctttgttatttttatcccacttcatgggagatattcatcagcctatgcatgttggatttacaagtgatatgggaggaaacagtatagatttgcgctggtttcgccacaaatccaacctgcaccatgtttgggatagagagattattcttacagctgcagcagattaccatggtaaggatatgcactctctcctacaagacatacagaggaactttacagagggtagttggttgcaagatgttgaatcctggaaggaatgtgatgatatctctacttgcgccaataagtatgctaaggagagtataaaactagcctgtaactggggttacaaagatgttgaatctggcgaaactctgtcagataaatacttcaacacaagaatgccaattgtcatgaaacggatagctcagggtggaatccgtttatccatgattttgaaccgagttcttggaagctccgcagatcattctttggcatga

SEQ ID NO: 4 - DNA, 915 bp, Apium graveolens
atgacgcgattatattctgtgttctttcttttgttggctcttgtagttgaaccgggtgttagagcctggagcaaagaaggccatgtcatgacatgtcaaattgcgcaggatctgttggagccagaagcagcacatgctgtaaagatgctgttaccggactatgctaatggcaacttatcgtcgctgtgtgtgtggcctgatcaaattcgacactggtacaagtacaggtggactagctctctccatttcatcgatacacctgatcaagcctgttcatttgattaccagagagactgtcatgatccacatggagggaaggacatgtgtgttgctggagccattcaaaatttcacatctcagcttggacatttccgccatggaacatctgatcgtcgatataatatgacagaggctttgttatttttatcccacttcatgggagatattcatcagcctatgcatgttggatttacaagtgatatgggaggaaacagtatagatttgcgctggtttcgccacaaatccaacctgcaccatgtttgggatagagagattattcttacagctgcagcagattaccatggtaaggatatgcactctctcctacaagacatacagaggaactttacagagggtagttggttgcaagatgttgaatcctggaaggaatgtgatgatatctctacttgcgccaataagtatgctaaggagagtataaaactagcctgtaactggggttacaaagatgttgaatctggcgaaactctgtcagataaatacttcaacacaagaatgccaattgtcatgaaacggatagctcagggtggaatccgtttatccatgattttgaaccgagttcttggaagctccgcagatcattctttggcaggaggtcaccatcaccatcaccattga

SEQ ID NO: 5 - DNA, 717 bp, Artificial Sequence
  This construct was derived by GRAMMR shuffling in accordance with the methodogy of the present invention.
atgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaaacggcatgactttttcaagagtgccatgcccgaaggttatgtacaggaacgcactatatttttcaaggatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataa

SEQ ID NO: 6 - DNA, 717 bp, Artificial Sequence
  This construct was derived by GRAMMR shuffling in accordance with the methodogy of the present invention.
atgagtaaaggagaagaacttttcactggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttctcttatggtgttcaatgcttttcaagatacccagatcatatgaaacggcatgactttttcaagagtgccatgcccgaaggttatgtacaggaacgcactatatctttcaaagatgacgggaactacaagacacgtgctgaagtcaagtttgaaggtgatacccttgttaatagaatcgagttaaaaggtattgattttaaagaagatggaaacattcttggacacaaactcgagtacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataa

SEQ ID NO: 7 - DNA, 717 bp, Artificial Sequence
  This construct was derived by GRAMMR shuffling in accordance with the methodogy of the present invention.
atgagtaaaggagaagaacttttcactggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatagaatcgagttaaaaggtattgattttaaagaagatggaaacattctcggacacaaattggaatacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgaactatacaaataa

SEQ ID NO: 8 - DNA, 717 bp, Artificial Sequence
  This construct was derived by GRAMMR shuffling in accordance with the methodogy of the present invention
atgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaaacggcatgactttttcaagagtgccatgcccgaaggttatgtacaggaaagaactatatttttcaaggatgacgggaactacaagacacgtgctgaagtcaagtttgaaggtgatacccttgttaatagaatcgagttaaaaggtattgattttaaagaagatggaaacattctcggacacaaactcgagtacaactataactcacacaatgtatacatcatggcagacaaacaaaagaatggaatcaaagttaacttcaaaattcgccacaacattgaagatggatccgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataa

SEQ ID NO: 9 - DNA, 807 bp, Tobacco mosaic virus
atggctctagttgttaaaggaaaagtgaatatcaatgagtttatcgacctgacaaaaatggagaagatcttaccgtcgatgtttacccctgtaaagagtgttatgtgttccaaagttgataaaataatggttcatgagaatgagtcattgtcaggggtgaaccttcttaaaggagttaagcttattgatagtggatacgtctgtttagccggtttggtcgtcacgggcgagtggaacttgcctgacaattgcagaggaggtgtgagcgtgtgtctggtggacaaaaggatggaaagagccgacgaggccactctcggatcttactacacagcagctgcaaagaaaagatttcagttcaaggtcgttcccaattatgctataaccacccaggacgcgatgaaaaacgtctggcaagttttagttaatattagaaatgtgaagatgtcagcgggtttctgtccgctttctctggagtttgtgtcggtgtgtattgtttatagaaataatataaaattaggtttgagagagaagattacaaacgtgagagacggagggcccatggaacttacagaagaagtcgttgatgagttcatggaagatgtccctatgtcgatcaggcttgcaaagtttcgatctcgaaccggaaaaaagagtgatgtccgcaaagggaaaaatagtagtagtgatcggtcagtgccgaacaagaactatagaaatgttaaggattttggaggaatgagttttaaaaagaataatttaatcgatgatgattcggaggctactgtcgccgaatcggattcgttttaa

SEQ ID NO: 10 - DNA, 793 bp, Tomato mosaic virus
atggctctagttgttaaaggtaaggtaaatattaatgagtttatcgatctgtcaaagtctgagaaacttctcccgtcgatgttcacgcctgtaaagagtgttatggtttcaaaggttgataagattatggtccatgaaaatgaatcattgtctgaagtaaatctcttaaaaggtgtaaaacttatagaaggtgggtatgtttgcttagttggtcttgttgtgtccggtgagtggaatttaccagataattgccgtggtggtgtgagtgtctgcatggttgacaagagaatggaaagagcggacgaagccacactggggtcatattacactgctgctgctaaaaagcggtttcagtttaaagtggtcccaaattacggtattactacaaaggatgcagaaaagaacatatggcaggtcttagtaaatattaaaaatgtaaaaatgagtgcgggctactgccctttgtcattagaatttgtgtctgtgtgtattgtttataaaaataatataaaattgggtttgagggagaaagtaacgagtgtgaacgatggaggacccatggaactttcagaagaagttgttgatgagttcatggagaatgttccaatgtcggttagactcgcaaagtttcgaaccaaatcctcaaaaagaggtccgaaaaataataataatttaggtaaggggcgttcaggcggaaggcctaaaccaaaaagttttgatgaagttgaaaaagagtttgataatttgattgaagatgaagccgagacgtcggtcgcggattctgattcgtatt

SEQ ID NO: 11 - DNA, 795 bp, Artificial Sequence
  This construct was derived by GRAMMR shuffling in accordance with the methodogy of the present invention
atggctctagttgttaaaggtaaggtaaatattaatgagtctatcgatctgtcaaagtctgagaaacttctcccgtcgatgttcacgcctgtaaagagtgttatggtttcaaaggttgataagattatggtccatgaaaatgaatcattgtctgaagtaaatctcttaaaaggtgtaaaacttatagaaggtgggtatgtttgcttagttggtcttgttgtgtccggtgagtggaatttaccagataattgccgtggtggtgtgagtgtctgcatggttgacaagagaatggaaagagcggacgaagccacactggggtcatattacactgctgctgctaaaaagcggtttcagttcaaggtcgttcccaattatgctataaccacccaggatgcagaaaagaacatatggcaggtcttagtaaatattaaaaatgtaaaaatgagtgcgggctactaccctttgtcattagaatttgtgtctgtgtgtattgtttataaaaataatataaaattgggtttgagggagaaagtaacgagtgtgaacgatggaggacccatggaactttcagaagaagttgttgatgagttcatggagaatgttccaatgtcgatcaggcttgcaaagtttcgaaccaaatcctcaaaaagaggtccgaaaaataataataatttaggtaaggggcgttcaggcggaaggcctaaaccaagaagttttgatgaagttgaaaaagagtttgataatttgattgaagatgaagccgagacgtcggtcgcggattctgattcgtattaa

SEQ ID NO: 12 - DNA, 795 bp, Artificial Sequence
  This construct was derived by GRAMMR shuffling in accordance with the methodogy of the present invention.
atggctctagttgttaaaggtaaggtaaatattaatgagtttatcgatctgtcaaagtctgagaaacttctcccgtcgatgttcacgcctgtaaagagtgttatggtttcaaaggttgataagattatggtccatgaaaatgaatcattgtctgaagtaaatctcttaaaaggtgtaaaacttatagaaggtgggtatgtttgcttagttggtcttgttgtgtccggtgtgtggaatttaccagataattgccgtggtggtgtgagtgtctgcatggttgacaagagaatggaaagagcggacgaggccacactcggatcttactacactgctgctgctaaaaagcggtttcagttcaaggtcgttcccaattatgctataaccacccaggatgcagaaaagaacatatggcaggtcttagtaaatattaaaaatgtaaaaatgagtgcgggctactgccctttgtcattagaatttgtgtctgtgtgtattgtttataaaaataatataaaattgggtttgagggagaaagtaacgagtgtgaacgatggaggacccatggaactttcagaagaagttgttgatgagttcatggagaatgttccaatgtcggttagactcgcaaagtttcgaaccaaatcctcaaaaagaggtccgaaaaataataataatttaggtaaggggcgttcaggcggaaggcctaaaccaaaaagttttgatgaagttggaaaagagtttgataatttgattgaagatgaagccgagacgtcggtcgcggattctgattcgtattaa

SEQ ID NO: 13 - DNA, 795 bp, Artificial Sequence
  This construct was derived by GRAMMR shuffling in accordance with the methodogy of the present invention.
atggctctagttgttaaaggtaaggtaaatattaatgagtttatcgatctgtcaaagtctgagaaacttctcccgtcgatgttcacgcctgtaaggagtgttatggtttcaaaggttgataagattatggtccatgaaaatgaatcattgtctgaagtaaatctcttaaaaggtgtaaaacttatagaaggtgggtatgtttgcttagttggtcttgttgtgtccggtgagtggaatttaccagataattgccgtggtggtgtgagtgtctgcatggttgacaagagaatggaaagagcggacgaagccacactggggtcatattacactgctgctgctaaaaagcggtttcagtttaaagtggtcccaaattacggtattactacccaggacgcgatgaaaaacgtctggcaggtcttagtaaatattaaaaatgtaaaaatgagtgcgggctactgccctttgtcattagaatttgtgtctgtgtgtattgtttataaaaataatataaaattgggtttgagggagaaagtaacgagtgtgaacgatggaggacccatggaactttcagaagaagttgttgatgagttcatggagaatgttccaatgtcgatcagactcgcaaagtttcgaaccaaatcctcaaaaagaggtccgaaaaataataataatttaggtaaggggcgttcaggcggaaggcctaaaccaaaaagttttgatgaagttgaaaaagagtttgataatttgattgaagatgaagccgagacgtcggtcgcggattctgattcgtattaa

SEQ ID NO: 14 - DNA, 796 bp, Artificial Sequence
  This construct was derived by GRAMMR shuffling in accordance with the methodogy of the present invention.
atggctctagttgttaaaggtaaggtaaatattaatgagtttatcgatctgtcaaagtctgagaaacttctcccgtcgatgttcacgcctgtaaagagtgttatggtttcaaaggttgataagattatggtccatgaaaatgaatcattgtctgaagtaaatctcttaaaaggtgttaagcttattgatagtggatacgtctgtttagccggtttggtcgtcacgggcgagtggaatttaccagataattgccgtggtggtgtgagtgtctgcatggttgacaagagaatggaaagagcggacgaagccacactggggtcatattacactgctgctgctaaaaagcggtttcagttcaaggtcgttcccaaattacggtattactacccaggatgcagaaaagaacatatggcaggtcttagtaaatattaaaaatgtaaaaatgagtgcgggctactgcccgctttctctggagtttgtgtctgtgtgtattgtttataaaaataatataaaattgggtttgagggagaaagtaacgagtgtgaacgatggaggacccatggaactttcagaagaagttgttgatgagttcatggagaatgttccaatgtcggttagactcgcaaagtttcgaaccaaatcctcaaaaagaggtccgaaaaataataataatttaggtaaggggcgttcaggcggaaggcctaaaccaaaaagttttgatgaagttgaaaaagagtttgataatttgattgaggatgattcggaggctactgtcgccgattctgattcgtattaa

SEQ ID NO: 15 - DNA, 795 bp, Artificial Sequence
  This construct was derived by GRAMMR shuffling in accordance with the methodogy of the present invention.
atggctctagttgttaaaggaaaagtgaatattaatgagtttatcgatctgtcaaagtctgagaaacttctcccgtcgatgttcacgcctgtaaagagtgttatggtttcaaaggttgataagattatggtccatgaaaatgaatcattgtctgaagtaaatctcttaaaaggtgtaaaacttatagaaggtgggtatgtttgcttagttggtcttgttgtgtccggcgagtggaatttaccagataattgccgtggtggtgtgagtgtctgcatggttgacaagagaatggaaagagcggacgaagccacactggggtcatattacactgctgctgcaaagaaaagatttcagttcaaggtcgttcccaattatgctataaccacccaggatgcagaaaagaacatatggcgggtcttagtaaatattaaaaatgtaaaaatgagtgcgggctactgcccgctttctctggagtttgtgtctgtgtgtattgtttataaaaataatataaaattgggtttgagggagaaagtaacgagtgtgaacgatgaaggacccatggaactttcagaagaagttgttgatgagttcatggagaatgttccaatgtcgatcaggctcgcaaagtttcgaaccaaatcctcaaaaagaggtccgaaaaataataataatttaggtaaggggcgttcaggcggaaggcctaaaccaaaaagttttgatgaagttgaaaaagagtttgataatttgattgaagatgaagccgagacgtcggtcgcggattctgattcgtactaa

SEQ ID NO: 16 - DNA, 888 bp, Selaginella lepidophylla
atggcaacgaccaagacgagcgggatggcgctggctttgctcctcgtcgccgccctggccgtgggagctgcggcctgggggaaagagggccatcgcctcacttgtatggtcgccgagccctttctaagctctgaatccaagcaagctgtggaggagcttctctctggaagagatctcccggacttgtgttcatgggccgatcagattcgaagatcgtataagtttagatggactggtcctttgcactacatcgatactccagacaacctctgcacctatgactatgatcgtgactgccacgattcccatgggaagaaggacgtgtgtgtcgctggtgggatcaacaattactcgtcgcagctggaaacgtttctagattcagagagctcgtcgtataacttgaccgaggcgctgctcttcctggctcactttgtcggggatatacaccagcccttgcacgtagcatttacgagtgatgccggaggcaatggcgtgcacgtccgctggtttggacgaaaggccaacttgcatcacgtctgggatacagaatttatttctagagccaatcgtgtgtactaccacgacatttccaagatgctccggaacattaccaggagcataactaagaagaatttcaatagttggagcagatgtaagactgatccggcggcttgtattgatagttatgcgacagaaagtatagatgcttcttgcaactgggcatacaaagacgcacccgacggaagctctctagatgatgattacttctcttcacgccttccaattgttgagcagcgtcttgctcaagggggcgtcaggctggcgtcaatactcaacaggatttttggaggagcaaagtcgaacaggtccagtcgctcaagcatgtag

SEQ ID NO: 17 - DNA, 3637 bp, Artificial Sequence
  Encodes cycle 3 GFP
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgggtaccgatgagtaaaggagaagaacttttcactggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgaactatacaaataagaattcctgcagcccgggggatccactagttctagagcggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 18 - DNA, 796 bp, Tobamovirus Cg
atgtcttacgagcctaaagtgagcgacttccttgctcttacgaaaaaggaggaaattttacccaaggctcttacgaggttaaagactgtctctattagtactaaggatgttatatctgttaaggattctgagtccctgtgtgatatagatttactagttaatgtgccattagataagtatagatatgtgggtgttttaggtgttgtttttaccggtgagtggttagtgccggatttcgttaaaggtggagtaacagtgagcgtgattgacaaacggcttgagaactccaaagagtgcataattggtacgtacagagctgctgcgaaagacaaaaggttccagttcaagctggttccaaattacttcgtgtctgttgcagatgccaagcgaaaaccgtggcaagttcatgtgcgtattcaaaatttaaggattgaagctggatggcaacctctggccttagaggtggtttctgttgctatggtcactaataacgtggttgttaagggtttgagagaaaaggtcatcgcagtgaatgatccgaatgtcgaaggtttcgaaggcgtggttgacgatttcgtcgattcggtcgcagcattcaaggcggttgacactttcagaaagaaaaagaaaaggattggaggaaaggatgtaaataataataagtttagatatagaccggagagatacgccggtcaggattcgttaaattataaagaagaaaacgtcttacaacatcacgaactcgaatcagtaccagtatttcgcagcgacgtgggcagagcccacagcgatgctt

SEQ ID NO: 19 - DNA, 823 bp, Tobamovirus Ob
atgtcaaaggctattgtcaagatcgatgaattcattaaattatccaagtctgaagaggttttaccttctgcattcacaagaatgaagtcggtcagagtctcaacagtggataagataatggccaaagagaatgacaatatttccgaagtagatttacttaagggtgttaagttagttaaaaatggttatgtttgtttagtaggtcttgtggtgtcaggagagtggaatttacccgacaactgcagaggtggtgtaagtatctgtctgatagacaaacgtatgcaacgtcataacgaagctactttaggttcgtacactaccaaagccagcaagaaaaacttttcgttcaagcttataccgaattactcgataacctctcaagatgctgaaaggcgtccttgggaagttatggtaaatattcgtggtgtggctatgtccgaaggttggtgtccattatccttagagttcgtttctgtttgtattgttcataaaaacaatgttagaaagggtctaagagagaaggtgactgccgtgtccgaagacgacgctatagaactcacagaagaggttgttgatgagtttatagaagccgtaccgatggcgcgacgtttgcagaacttgagaaaacccaagtacaacaaagaaaaagaaaataaaaatttgaataataaaaatagtataggagtttccaaacctgtcggtttggaaagaaataaagtaaggagtgtagttagaaaaggggttaggagtgatagtagtttaggtgtgactgatatgagtcaggacggtagctcaagcgagatatcatccgattcgtttattt

SEQ ID NO: 20 - DNA, 769 bp, Tobacco mosaic virus-U2
atggctgttagtctcagagatactgtcaaaattagcgagttcattgatctttcgaaacaggatgagatacttccggcattcatgactaaggtcaagagcgtcagaatatcgactgtggacaagattatggctgttaagaatgatagtctttctgatgtagatttacttaaaggtgttaagttagttaagaatgggtacgtgtgcttagctggtttggtagtgtctggggagtggaatctcccggacaactgccgtggtggtgtcagtgtttgtattgtagataagagaatgaaaaggagtaaggaggcaacgctgggtgcgtatcacgcccctgcttgcaaaaagaatttttcctttaagctaatccctaattattcaataacatccgaggatgctgagaagcacccatggcaagtattagtgaatatcaaaggagtggctatggaagaaggatactgtcctttatctttggagttcgtttcaatttgtgtagtacataaaaataatgtaagaaaaggtttgagggaacgtattttgagagtaacagacggctcgccaattgaactcactgaaaaagttgttgaggagttcatagatgaagtaccaatggctgtgaaactcgaaaggttccggaaaacaaaaaagagagtggtaggtaatagtgttaataataagaaaataaataatagtggtaagaaaggtttgaaagttgaggaaattgaggataatgtaagtgatgacgagtctatcgcgtcatcgagtacgtttt

SEQ ID NO: 21 - DNA, 808 bp, Artificial Sequence
  This construct was derived by GRAMMR shuffling in accordance with the methodogy of the present invention.
atggctctagttgttaaaggtaaggtaaatattaatgagtttatcgatctgtcaaagtctgagaaacttctcccgtcgatgttcacgcctgtaaagagtgttatggtttcaaaggttgataagattatggtccatgaaaatgaatcattgtctgaagtaaatctcttaaaaggtgtaaaacttatagaaggtgggtatgtttgcttagttggtcttgttgtgtccggtgagtggaatttaccagataattgccgtggtggtgtgagtgtctgcatggttgacaagagaatggaaagagcggacgaagccacactggggtcatattacactgctgctgctaaaaagcggtttcagtttaaagtggtcccaaattacggtattactacaaaggatgcagaaaagaacatatggcaagttcatgtgcgtattcaaaatttaaggattgaagctggatggcaacctctggccttagaggtggtttctgttgctatggtcactaataacgtggttgttaagggtttgagagaaaaggtcatcgcagtgaatgatccgaatgtcgaaggtttcgaaggcgtggttgacgatttcgtcgattcggtcgcagcattcaaggcggttgacactttcagaaagaaaaagaaaaggattggaggaaaggatgtaaataataataagtttagatatagaccggagagatacgccggtcaggattcgttaaattataaagaagaaaacgtcttacaacatcacgaactcgaatcagtaccagtatttcgcagcgacgtgggcagagcccacagcgatgctt

SEQ ID NO: 22 - DNA, 799 bp, Artificial Sequence
  This construct was derived by GRAMMR shuffling in accordance with the methodogy of the present invention.
atgtcttacgagcctaaagtgagcgacttccttgctcttacgaaaaaggaggaaattttacccaaggctcttacgaggttaaagactgtctctattagtactaaggatgttatatctgttaaggattctgagtccctgtgtgatatagatttactagttaatgtgccattagataagtatagatatgtgggtgttttaggtgttgtttttaccggtgagtggaatttaccagataattgccgtggtggtgtgagtgtctgcatggttgacaagagaatggaaagagcggacgaagccacactggggtcatattacactgctgctgcgaaagacaaaaggttccagttcaagctggttccaaattacttcgtgtctgttgcagatgccaagcgaaaaccgtggcaagttcatgtgcgtattcaaaatttaaggattgaagctggatggcaacctctggccttagaggtggtttctgttgctatggtcactaataacgtggttgttaagggtttgagagaaaaggtcatcgcagtgaatgatccgaatgtcgaaggtttcgaaggcgtggttgacgatttcgtcgattcggtcgcagcattcaaggcggttgacactttcagaaagaaaaagaaaaggattggaggaaaggatgtaaataataataagtttagatatagaccggagagatacgccggtcaggattcgttaaattataaagaagaaaacgtcttacaacatcacgaactcgaatcagtaccagtatttcgcagcgacgtgggcagagcccacagcgatgctt

SEQ ID NO: 23 - DNA, 823 bp, Artificial Sequence
  This construct was derived by GRAMMR shuffling in accordance with the methodogy of the present invention.
aaataaacgaatcggatgatatctcgcttgagctaccgtcctgactcatatcagtcacacctaaactactatcactcctaaccccttttctaactacactccttactttatttctttccaaaccgacaggtttggaaactcctatactatttttattattcaaatttttattttctttttctttgttgtacttgggttttctcaagttctgcaaacgtcgcgccatcggtacggcttctataaactcatcaacaacctcttctgtgagttctatagcgtcgtcttcggacacggcagtcaccttctctcttagaccctttctaacattgtttttatgaacaatacaaacagaaacgaactctaaggataatggacaccaaccttcggacatagccacaccacgaatatttaccataacttcccaaggacgcctttcagcatcttgagaggttatcgagtaattcggtataagcttgaacgaaaagtttttcttgctggctttggtagtgtacgaacctaaagtagcttcgttatgacgttgcatacgtttgtctatcagacagatacttacaccacctctgcagttgtcgggtaaattccactctcctgacaccacaagacctactaaacaaacataaccaccttctataagttttacaccttttaagagatttacttcagacaatgattcattctctttggccattatcttatccactgttgagactctgaccgacttcattcttgtgaatgcagaaggtaaaacctcttcagacttggataatttaatgaattcatcgatcttgacaatagcctttgacat

SEQ ID NO: 24 - DNA, 792 bp, Artificial Sequence
  This construct was derived by GRAMMR shuffling in accordance with the methodogy of the present invention.
aatacgaatcagaatccgcgaccgacgtctcggcttcatcttcaatcaaattatcaaactctttttcaacttcatcaaaactttttggtttaggccttccgcctgaacgccccttacctaaattattattatttttcggacctctttttgaggatttggttcgaaactttgcgagtctaaccgacattggaacattctccatgaactcatcaacaacctcttctgtgagttctatagcgtcgtcttcggacacggcagtcaccttctctcttagaccctttctaacattgtttttatgaacaatacaaacagaaacgaactctaatgacaaagggcagtagcccgcactcatttttacatttttaatatttactaagacctgccatatgttcttttctgcatcctttgtagtaataccgtaatttgggaccactttaaactgaaaccgctttttagcagcagcagtgtaatatgaccccagtgtggcttcgtccgctctttccattctcttgtcaaccatgcagacactcacaccaccacggcaattatctggtaaattccactctcctgacaccacaagacctactaaacaaacataaccatttttaactaacttaacacccttaagagatttacttcggacaatgattcattttcatggaccataatcttatcaacctttgaaaccataacactctttacaggcgtgaatgcagaaggtaaaacctcttcagactttgacagatcgataaactcattaatatttaccttacctttaacaactagagccat

SEQ ID NO: 25 - DNA, 769 bp, Artificial Sequence
  This construct was derived by GRAMMR shuffling in accordance with the methodogy of the present invention.
aatacgaatcagaatccgcgatagactcgtcatcacttacattatcctcaatttcctcaactttcaaacctttcttaccactattatttattttcttattattaacactattacctaccactctcttttttgttttccggaacctttcgagtttcacagccattggtacttcatctatgaactcatcaacaacttcttctgaaagttccatgggtcctccatcgttcacactcgttactttctccctcaaacccaattttatattatttttataaacaatacacacagacacaaattctaaagataaagggcagtatccttcttccatagccactcctttgatattcactaatacttgccatgggtgcttttctgcatcctcggatgttattgaataattagggaccactttaaactgaaaccgctttttagcagcaggggcgtgatacgcacccagcgttgcctccttactcctttccattctcttgtcaaccatgcagacactcacaccaccacggcagttgtccgggagattccactcaccggacacaacaagaccaactaagcaaacatacccaccttctataagttttacaccttttaagagatttacttcagacaatgattcattttcatggaccataatcttatcaacctttgaaaccataacactctttacaggcgtgaacatcgacgggagaagtttctcagactttgacagatcgataaactcattaatatttaccttacctttaacaactagagccat

SEQ ID NO: 26 - DNA, 772 bp, Artificial Sequence
  This construct was derived by GRAMMR shuffling in accordance with the methodogy of the present invention.
aatacgaatcagaatccgcgaccgacgtctcggcttcacttacattatcctcaatttcctcaactttcaaaactttcttaccactattatttattttcttattattaacactattacctaccactctcttttttgttttccggaacctttcgagtttcacagccattggtacttcatctatgaactcatcaacaactttttcagtgagttcaattggcgagccgtctgttactctcaaaatacgttccctcaaacccaattttatattatttttataaacaatacacacagacacaaattctaatgacaaagggcagtagcccgcactcatttttacatttttaatatttactaagacctgccatgggtgcttctcagcatcctcggatgttattgaataattagggattagcttaaaggaaaaattctttttgcaagcaggggcgtgatacgcacccagtgtggcttcgtccgctctttccattctcttgtcaaccatgcagacactcacaccaccacggcagttgtccgggagattccactcaccggacacaacaagaccaactaagcacacgtacccattcttaactaacttaacacctttaagtaaatctacatcagacaatgattcattttcatggaccataatcttatcaacctttgaaaccataacactctttacaggcgtgaacatcgacgggagaagtttctcagactttgacagatcgataaactcgctaattttgacagtatctctgagactaacagccat

SEQ ID NO: 27 - DNA, 805 bp, Artificial Sequence
  This construct was derived by GRAMMR shuffling in accordance with the methodogy of the present invention.
atggctctagttgttaaaggaaaagtgaatattaatgagtttatcgatctgtcaaagtctgagaaacttctcccgtcgatgttcacgcctgtaaagagtgttatggtttcaaaggttgataagattatggtccatgaaaatgaatcattgtctgaagtaaatctcttaaaaggtgtaaaacttatagaaggtgggtatgtttgcttagttggtcttgttgtgtccggtgagtggaatttaccagataattgccgtggtggtgtgagtgtctgcatggttgacaagagaatggaaagagcggacgaagccactctcggatcttactacacagcagctgcaaagaaaagatttcagttcaaggtcgttcccaattatgctataaccacccaggacgcgatgaaaaacgtctggcaagttttagttaatattagaaatgtgaagatgtcagcgggtttctgtccgctttctctggagtttgtgtctgtgtgtattgtttataaaaataatataaaattgggtttgagggagaaagtaacgagtgtgaacgatggaggacccatggaactttcagaagaagttgttgatgagttcatggaagatgtcccaatgtcggttagactcgcaaagtttcgatctcgaaccggaaaaaagagtgatgtccgcaaagggaaaaatagtagtagtgatcggtcagtgccgaacaagaactatagaaatgttaaggattttggaggaatgagttttaaaaagaataatttaatcgatgatgattcggagacgtcggtcgcggattctgattcgtatt

SEQ ID NO: 28 - DNA, 804 bp, Artificial Sequence
  This construct was derived by GRAMMR shuffling in accordance with the methodogy of the present invention.
atggctctagttgttaaaggaaaagtgaatatcaatgagtttatcgacctgacaaagtctgagaaacttctcccgtcgatgtttacccctgtaaagagtgttatggttccaaagttgataagattatggttcatgagaatgagtcattgtcaggggtgaaccttcttaaaggagttaagcttattgatagtggatacgtctgtttagccggtttggtcgtcacgggcgagtggaacttgcctgacaattgccgtggtggtgtgagcgtgtgtctggtggacaagagaatggaaagagcggacgaagccacactggggtcatattacactgctgctgctaaaaagcggtttcagttcaaggtcgttcccaattatgctataaccacccaggatgcagaaaagaacatatggcaggtcttagtaaatattaaaaatgtgaagatgagtgcgggctactgccctttgtcattagaatttgtgtcggtgtgtattgtttatagaaataatataaaattgggtttgagagagaaagtaacgagtgtgaacgatggagggcccatggaacttacagaagaagtcgttgatgagttcatggaagatgtccctatgtcgatcaggcttgcaaagtttcgatctcgaatcctcaaaaagagtgatgtccgcaaagggaaaaatagtagtagtgatcggtcagtgccgaacaagaactatagaaatgttaaggattttggaggaatgagttttaaaaagaataatttaatcgatgatgattcggaggctactgtcgcggattctgattcgtttt

SEQ ID NO: 29 - DNA, 717 bp, Aequorea victoria GFP ORF
atgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaaacggcatgactttttcaagagtgccatgcccgaaggttatgtacaggaaagaactatatttttcaaggatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataa

SEQ ID NO: 30 - DNA, 717 bp, Aequorea victoria GFP Cycle 3 ORF
atgagtaaaggagaagaacttttcactggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgaactatacaaataa

SEQ ID NO: 31 - DNA, 3637 bp, Artificial Sequence
  Plasmid encoding wild type Aequorea victoria GFP Cycle 3 ORF
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgggtaccgatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaaacggcatgactttttcaagagtgccatgcccgaaggttatgtacaggaaagaactatatttttcaaggatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataagaattcctgcagcccgggggatccactagttctagagcggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 32 - DNA, 3637 bp, Artificial Sequence
  Encodes Cycle 3 BFP gene
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgggtaccgatgagtaaaggagaagaacttttcactggagttgtcccaattcttgttgaattagatggtgatgttaatgggcacaaattttctgtcagtggagagggtgaaggtgatgctacatacggaaagcttacacttaaatttatttgcactactggaaaactacctgttccatggccaacacttgtcactactttctctcatggtgttcaatgcttttctcgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactttaactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgaactatacaaataagaattcctgcagcccgggggatccactagttctagagcggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 33 - DNA, 717 bp, Aequorea victoria BFP Cycle 3 ORF
atgagtaaaggagaagaacttttcactggagttgtcccaattcttgttgaattagatggtgatgttaatgggcacaaattttctgtcagtggagagggtgaaggtgatgctacatacggaaagcttacacttaaatttatttgcactactggaaaactacctgttccatggccaacacttgtcactactttctctcatggtgttcaatgcttttctcgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactttaactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgaactatacaaataa

SEQ ID NO: 34 - protein, 295 aa, Selaginella lepidophylla
MATTKTSGMALALLLVAALAVGAAAWGKEGHRLTCMVAEPFLSSESKQAVEELLSGRDLPDLCSWADQIRRSYKFRWTGPLHYIDTPDNLCTYDYDRDCHDSHGKKDVCVAGGINNYSSQLETFLDSESSSYNLTEALLFLAHFVGDIHQPLHVAFTSDAGGNGVHVRWFGRKANLHHVWDTEFISRANRVYYHDISKMLRNITRSITKKNFNSWSRCKTDPAACIDSYATESIDASCNWAYKDAPDGSSLDDDYFSSRLPIVEQRLAQGGVRLASILNRIFGGAKSNRSSRSSM

SEQ ID NO: 35 - protein, 19 aa, Apium graveolens fragment of Cel I expressed by TMV
DMCVAGAIQNFTSQLGHFR

(19) United States
(12) Patent Application Publication
(10) Pub. No.: US 2004/0142433 A1
(43) Pub. Date: Jul. 22, 2004

(54) Polynucleotide sequence variants

(76) Inventors: Hal S. Padgett, Vacaville, CA (US); Wayne P. Fitzmaurice, Vacaville, CA (US); John A. Lindbo, Vacaville, CA (US); Andrew A. Vaewhongs, Vacaville, CA (US); Fakhrieh S. Vojdani, Davis, CA (US); Mark L. Smith, Davis, CA (US)

Correspondence Address: JONDLE & ASSOCIATES P.C., 9085 EAST MINERAL CIRCLE, SUITE 200, CENTENNIAL, CO, 80112, US

(21) Appl. No.: 10/684,134

(22) Filed: Oct. 10, 2003

Related U.S. Application Data
(63) Continuation-in-part of application No. 10/637,758, filed on Aug. 8, 2003.
(63) Continuation-in-part of application No. 10/226,372, filed on Aug. 21, 2002.
(63) Continuation-in-part of application No. 10/280,913, filed on Oct. 25, 2002.
(63) Continuation-in-part of application No. 10/066,390, filed on Feb. 1, 2002.
(60) Provisional application No. 60/402,342, filed on Aug. 8, 2002.
(60) Provisional application No. 60/268,785, filed on Feb. 14, 2001.
(60) Provisional application No. 60/266,386, filed on Feb. 2, 2001.

(51) Int. Cl. C12P 19/34; A61K 48/00

(52) U.S. Cl. 435/91.2; 514/44

(57) ABSTRACT

We describe here an in vitro method of redistributing sequence variations between non-identical polynucleotide sequences, by making a heteroduplex polynucleotide from two non-identical polynucleotides; introducing a nick in one strand at or near a base pair mismatch site; removing mismatched base(s) from the mismatch site where the nick occurred; and using the opposite strand as template to replace the removed base(s) with bases that complement base(s) in the first strand. By this method, information is transferred from one strand to the other at sites of mismatch.

This application is a continuation-in-part of U.S. application Ser. No. 10/637,758, filed Aug. 8, 2003, which claims priority to U.S. Provisional Application No. 60/402,342, filed Aug. 8, 2002; U.S. application Ser. No. 10/226,372, filed Aug. 21, 2002, U.S. application Ser. No. 10/280,913 filed Oct. 25, 2002 and U.S. application Ser. No. 10/066,390, filed Feb. 1, 2002, which claims priority to U.S. Provisional Application No. 60/268,785, filed Feb. 14, 2001 and U.S. Provisional Application No. 60/266,386, filed Feb. 2, 2001, and all of which are incorporated herein by reference.

### FIELD OF THE INVENTION

The invention relates generally to molecular biology and more specifically to methods of generating populations of related nucleic acid molecules.

### BACKGROUND INFORMATION

DNA shuffling is a powerful tool for obtaining recombinants between two or more DNA sequences to evolve them in an accelerated manner. The parental, or input, DNAs for the process of DNA shuffling are typically mutants or variants of a given gene that have some improved character over the wild-type. The products of DNA shuffling represent a pool of essentially random reassortments of gene sequences from the parental nucleic acids that can then be analyzed for additive or synergistic effects resulting from new sequence combinations.

Recursive sequence reassortment is analogous to an evolutionary process where only variants with suitable properties are allowed to contribute their genetic material to the production of the next generation. Optimized variants are generated through DNA shuffling-mediated sequence reassortment followed by testing for incremental improvements in performance. Additional cycles of reassortment and testing lead to the generation of genes that contain new combinations of the genetic improvements identified in previous rounds of the process. Reassorting and combining beneficial genetic changes allows an optimized sequence to arise without having to individually generate and screen all possible sequence combinations.

This differs sharply from random mutagenesis, where subsequent improvements to an already improved sequence result largely from serendipity. For example, in order to obtain a protein that has a desired set of enhanced properties, it may be necessary to identify a mutant that contains a combination of various beneficial mutations. If no process is available for combining these beneficial genetic changes, further random mutagenesis will be required. However, random mutagenesis requires repeated cycles of generating and screening large numbers of mutants, resulting in a process that is tedious and highly labor intensive. Moreover, the rate at which sequences incur mutations with undesirable effects increases with the information content of a sequence. Hence, as the information content, library size, and mutagenesis rate increase, the ratio of deleterious mutations to beneficial mutations will increase, increasingly masking the selection of further improvements. Lastly, some computer simulations have suggested that point mutagenesis alone may often be too gradual to allow the large-scale block changes that are required for continued and dramatic sequence evolution.

There are a number of different techniques used for random mutagenesis. For example, one method utilizes error-prone polymerase chain reaction (PCR) for creating mutant genes in a library format, (Cadwell and Joyce, 1992; Gram et al., 1992). Another method is cassette mutagenesis (Arkin and Youvan, 1992; Delagrave et al., 1993; Delagrave and Youvan, 1993; Goldman and Youvan, 1992; Hermes et al., 1990; Oliphant et al., 1986; Stemmer et al., 1993) in which the specific region to be optimized is replaced with a synthetically mutagenized oligonucleotide.

Error-prone PCR uses low-fidelity polymerization conditions to introduce a low level of point mutations randomly over a sequence. A limitation to this method, however, is that published error-prone PCR protocols suffer from a low processivity of the polymerase, making this approach inefficient at producing random mutagenesis in an average-sized gene.

In oligonucleotide-directed random mutagenesis, a short sequence is replaced with a synthetically mutagenized oligonucleotide. To generate combinations of distant mutations, different sites must be addressed simultaneously by different oligonucleotides. The limited library size that is obtained in this manner, relative to the library size required to saturate all sites, requires that many rounds of selection are required for optimization. Mutagenesis with synthetic oligonucleotides requires sequencing of individual clones after each selection round followed by grouping them into families, arbitrarily choosing a single family, and reducing it to a consensus motif. Such a motif is resynthesized and reinserted into a single gene followed by additional selection. This step creates a statistical bottleneck, is labor intensive, and is not practical for many rounds of mutagenesis.

For these reasons, error-prone PCR and oligonucleotide-directed mutagenesis can be used for mutagenesis protocols that require relatively few cycles of sequence alteration, such as for sequence fine-tuning, but are limited in their usefulness for procedures requiring numerous mutagenesis and selection cycles, especially on large gene sequences.

As discussed above, prior methods for producing improved gene products from randomly mutated genes are of limited utility. One recognized method for producing a randomly reasserted gene sequences uses enzymes to cleave a long nucleotide chain into shorter pieces. The cleaving agents are then separated from the genetic material, and the material is amplified in such a manner that the genetic material is allowed to reassemble as chains of polynucleotides, where their reassembly is either random or according to a specific order. The method requires several rounds of amplification to assemble variants of genes that were broken into random fragments. ((Stemmer, 1994a; Stemmer, 1994b), U.S. Pat. No. 5,605,793, U.S. Pat. No. 5,811,238, U.S. Pat. No. 5,830,721, U.S. Pat. No. 5,928,905, U.S. Pat. No. 6,096,548, U.S. Pat. No. 6,117,679, U.S. Pat. No. 6,165,793, U.S. Pat. No. 6,153,410). A variation of this method uses primers and limited polymerase extensions to generate the fragments prior to reassembly (U.S. Pat. No. 5,965,408, U.S. Pat. No. 6,159,687).

However, both methods have limitations. These methods suffer from being technically complex. This limits the applicability of these methods to facilities that have sufficiently experienced staffs. In addition there are complications that arise from the reassembly of molecules from fragments, including unintended mutagenesis and the increasing difficulty of the reassembly of large target molecules of increasing size, which limits the utility of these methods for reassembling long polynucleotide strands.

Another limitation of these methods of fragmentation and reassembly-based gene shuffling is encountered when the parental template polynucleotides are increasingly heterogeneous. In the annealing step of those processes, the small polynucleotide fragments depend upon stabilizing forces that result from base-pairing interactions to anneal properly. As the small regions of annealing have limited stabilizing forces due to their short length, annealing of highly complementary sequences is favored over more divergent sequences. In such instances these methods have a strong tendency to regenerate the parental template polynucleotides due to annealing of complementary single-strands from a particular parental template. Therefore, the parental templates essentially reassemble themselves creating a background of unchanged polynucleotides in the library that increases the difficulty of detecting recombinant molecules. This problem becomes increasingly severe as the parental templates become more heterogeneous, that is, as the percentage of sequence identity between the parental templates decreases. This outcome was demonstrated by Kikuchi, et al., (Gene 243:133-137, 2000) who attempted to generate recombinants between xylE and nahH using the methods of family shuffling reported by Patten et al., 1997; Crameri et al., 1998; Harayama, 1998; Kumamaru et al., 1998; Chang et al., 1999; Hansson et al., 1999). Kikuchi, et al., found that essentially no recombinants (<1%) were generated. They also disclosed a method to improve the formation of chimeric genes by fragmentation and reassembly of single-stranded DNAs. Using this method, they obtained chimeric genes at a rate of 14 percent, with the other 86 percent being parental sequences.

The characteristic of low-efficiency recovery of recombinants limits the utility of these methods for generating novel polynucleotides from parental templates with a lower percentage of sequence identity, that is, parental templates that are more diverse. Accordingly, there is a need for a method of generating gene sequences that addresses these needs.

The present invention provides a method that satisfies the aforementioned needs, and provides related advantages as well.

### SUMMARY OF THE INVENTION

The present invention provides a method for reassorting mutations among related polynucleotides, in vitro, by forming heteroduplex molecules and then addressing the mismatches such that sequence information at sites of mismatch is transferred from one strand to the other. In one preferred embodiment, the mismatches are addressed by incubating the heteroduplex molecules in a reaction containing a mismatch nicking enzyme, a polymerase with a 3′ to 5′ proofreading activity in the presence of dNTPs, and a ligase. These respective activities act in concert such that, at a given site of mismatch, the heteroduplex is nicked, unpaired bases are excised from one of the strands, then replaced using the opposite strand as a template, and nicks are sealed. Output polynucleotides may be amplified before cloning, or cloned directly and tested for improved properties. Additional cycles of mismatch resolution reassortment and testing may lead to further improvement.

In one embodiment, an in vitro method of increasing homogeneity between two strands of the heteroduplex polynucleotide sequence, the method includes mixing the heteroduplex polynucleotide sequence with an effective amount of mismatch-directed strand cleavage activity, proofreading activity, and ligase activity; and allowing sufficient time for homogeneity between two strands of the heteroduplex polynucleotide sequence to result.

In another embodiment an in vitro method of increasing the number of complementary base pairs in a heteroduplex polynucleotide sequence where said heteroduplex polynucleotide sequence has at least two non-complementary nucleotide base pairs, the method includes mixing the heteroduplex polynucleotide sequence with an effective amount of mismatch-directed strand cleavage activity, proofreading activity, and ligase activity; and allowing sufficient time for a number of non-complementary nucleotide base pairs to be converted to complementary base pairs, wherein the homogeneity between the strands is increased by at least one complementary base pair.

In another embodiment an in vitro method of making a population of sequence variants from a heteroduplex polynucleotide sequence wherein said heteroduplex polynucleotide sequence has at least two non-complementary nucleotide base pairs, said method includes mixing copies of the heteroduplex polynucleotide sequence ith an effective amount of mismatch-directed strand cleavage activity, proofreading activity, and ligase activity; and allowing sufficient time for a number of non-complementary nucleotide base pairs to be converted to complementary base pairs, wherein a diverse population of polynucleotide sequences results.

In another embodiment an in vitro method of obtaining a polynucleotide sequence encoding a desired functional property, includes preparing at least one heteroduplex polynucleotide sequence; mixing copies of the heteroduplex polynucleotide sequence with an effective amount of mismatch-directed strand cleavage activity, proofreading activity, and ligase activity; and allowing sufficient time for the percentage of complementarity between strands of the heteroduplex polynucleotide sequence to increase, wherein sequence diversity in the population is increased; and screening or selecting a population of variants for the desired functional property.

In another embodiment an in vitro method of obtaining a polynucleotide encoding a desired functional property, includes preparing at least one heteroduplex polynucleotide, mixing copies of the heteroduplex polynucleotide sequence with an effective amount of mismatch-directed strand cleavage activity, proofreading activity, and ligase activity, allowing sufficient time for some or all of the mismatched nucleotide base pairs in the heteroduplex polynucleotide sequence to be converted to complementary bases, wherein a diverse population of polynucleotide sequences results, screening or selecting for a population of variants having a desired functional property, denaturing said population of variants to obtain a population of single stranded polynucleotide sequences, annealing said population of single stranded polynucleotide sequences to form a diverse population heteroduplex polynucleotide sequences, mixing the heteroduplex polynucleotide sequence with an effective amount of mismatch-directed strand cleavage activity, proofreading activity, and ligase activity, allowing sufficient time for some or all of the mismatched nucleotide base pairs in the heteroduplex polynucleotide sequence to be converted to matched base pairs, wherein a diverse population of polynucleotide sequences results, and screening or selecting for a population of variants having a desired functional property. DNA can be converted to RNA prior to screening by transcription of the DNA. A ligase activity can be added to seal the strands after proofreading.

One of the advantages of this method is that the sequence is either circular or linear. This allows for shuffling of nearly unlimited sequence length. The variant polynucleotide sequences have different amounts of complementarity. In the examples below, data is presented showing increasing the complementarity in a polynucleotide heteroduplex between two polynucleotides with sequence homology as low as 47%.

One advantage of the present invention over previous gene shuffleing methods such as that of Stemmer et al, is the ability to exchange sequences within an area of high occurrences of mismatches. Because the method of Stemmer et al requires reannealing of fragments, a considerably amount of identity is required, generally at least about 70%. The present invention is capable of cleaving and resolving in regions of much lower identity because the entire polynucleotide is generally merely nicked and held together than double stranded cleaved, denatured and reanealed.

This process can occur simultaneously at many sites and on either strand of a given heteroduplex DNA molecule. The result is a randomization of sequence differences among input strands to give a population of sequence variants that is more diverse than the population of starting sequences.

In another embodiment, a method of identifying a reasserted DNA molecule encoding a protein with a desired functional property, includes providing at least one single-stranded uracil-containing DNA molecule, which single-stranded uracil-containing DNA molecule, or a complementary strand thereto, encodes a protein; providing one or a plurality of non-identical single-stranded DNA molecules capable of hybridizing to the single-stranded uracil-containing DNA molecule, wherein said DNA molecules encode at least one additional variant of the protein; contacting the single-stranded uracil-containing DNA molecule with at least one single-stranded DNA molecule of step (b), thereby producing an annealed DNA molecule; incubating the annealed DNA molecule with a mismatch endonuclease, proofreading polymerase and a ligase, thereby producing a sequence-reassorted DNA strand annealed to the uracil-containing DNA molecule; amplifying the reassorted DNA strand under conditions wherein the uracil-containing DNA molecule is not amplified, thereby producing a population of reasserted DNA molecules; and, screening or selecting the population of reassorted DNA molecules to identify those that encode a polypeptide having the desired functional property, thereby identifying one or more DNA molecules(s) that encode a polypeptide with the desired functional property. This process can also occur using an RNA molecule as a template.

### BRIEF DESCRIPTION OF THE FIGURES

FIG. 1 depicts the process of Genetic Reassortment by Mismatch Resolution (GRAMMR). Reassortment is contemplated between two hypothetical polynucleotides differing at least two nucleotide positions. Annealing between the top strand of A and the bottom strand of B is shown which results in mismatches at the two positions. After the process of reassortment mismatch resolution, four distinct product polynucleotides are seen, the parental types A and B, and the reassorted products X and Y.

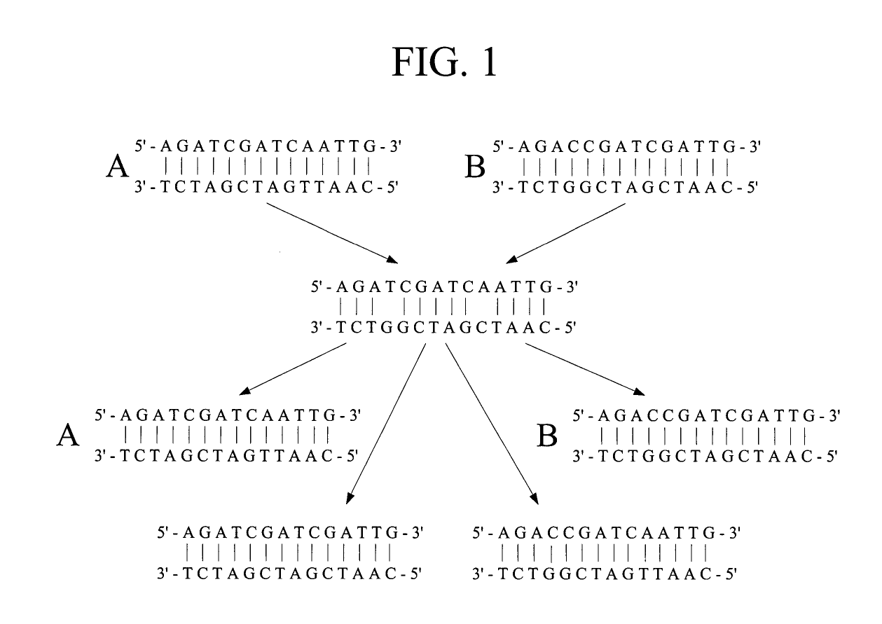

FIG. 2 depicts an exemplary partially complementary nucleic acid population of two molecules. FIG. 2A shows the sequence of two nucleic acid molecules “X” and “Y” having completely complementary top/bottom strands 1+/2− and 3+/4−, respectively. The positions of differing nucleotides between the nucleic acids X and Y are indicated (*). FIG. 2B shows possible combinations of single strands derived from nucleic acids X and Y after denaturing and annealing and indicates which of those combinations would comprise a partially complementary nucleic acid population of two.

FIG. 3 shows nucleic acid sequence for RES I endonuclease (SEQ ID NO: 16) as taught in Example 13.

FIG. 4 shows the corresponding amino acid sequence for RES I (SEQ ID NO: 34).

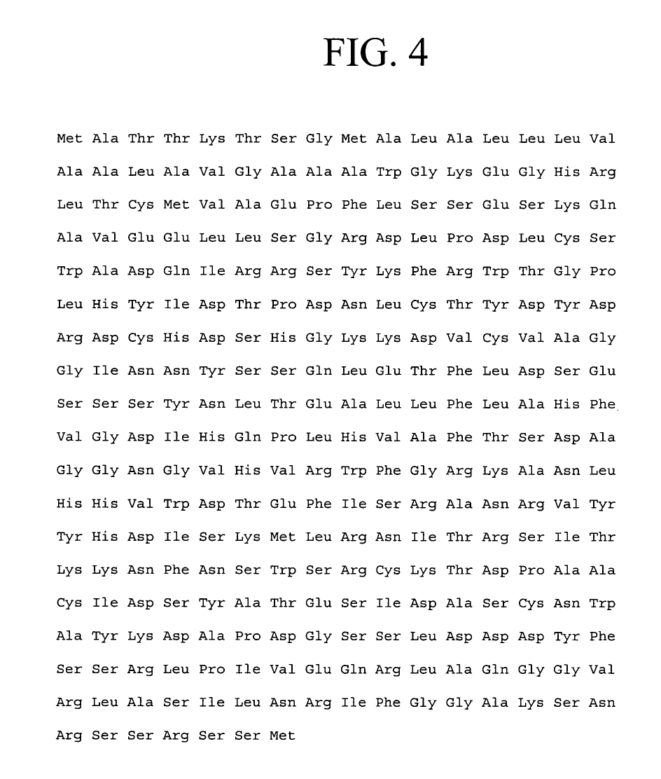

FIG. 5 shows the nucleic acid sequence for plasmid pBSC3BFP (SEQ ID NO: 32) as taught in Example 14.

FIG. 6 shows the nucleic acid sequence for the tobamovirus movement protein open reading frame of TMV-Cg (SEQ ID NO: 18) as taught in Example 15.

FIG. 7 shows the nucleic acid sequence for the tobamovirus movement protein open reading frame of TMV-Ob (SEQ ID NO: 19) as taught in Example 15.

FIG. 8 shows the nucleic acid sequence for the tobamovirus movement protein open reading frame of TMV-U2 (SEQ ID NO: 20) as taught in Example 15.

FIG. 9 shows a resultant clone from TMV-Cg and ToMv GRAMMR reaction (SEQ ID NO: 21) as taught in Example 15.

FIG. 10 shows a second resultant clone from a TMV-Cg and ToMv GRAMMR reaction (SEQ ID NO: 22) as taught in Example 15.

FIG. 11 shows a resultant clone from a TMV-Ob and ToMv GRAMMR reaction (SEQ ID NO: 23) as taught in Example 15.

FIG. 12 shows a second resultant clone from a TMV-Ob and ToMv GRAMMR reaction (SEQ ID NO: 24) as taught in Example 15.

FIG. 13 shows a resultant clone from a TMV-U2 and ToMv GRAMMR reaction (SEQ ID NO: 25) as taught in Example 15.

FIG. 14 shows a second resultant clone from a TMV-U2 and ToMv GRAMMR reaction (SEQ ID NO: 26) as taught in Example 15.

FIG. 15 shows a resultant clone from a TMV-U1 and ToMv GRAMMR reaction (SEQ ID NO: 27) as taught in Example 15.

FIG. 16 shows a second resultant clone from a TMV-U1 and ToMv GRAMMR reaction (SEQ ID NO: 28) as taught in Example 15.

FIG. 17 shows the nucleic acid sequence for the tobamovirus movement protein open reading frame of TMV (SEQ ID NO: 9) as taught in Example 15.

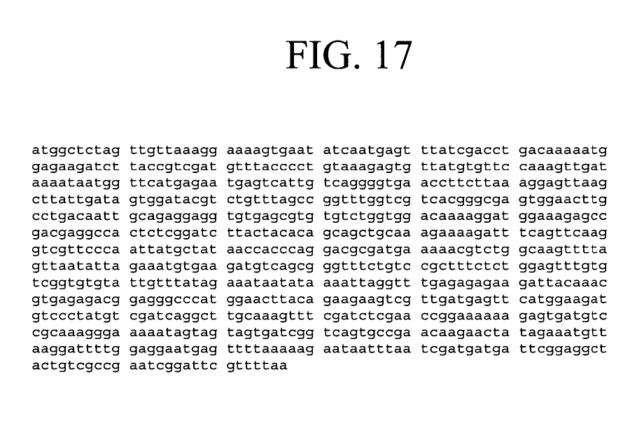

FIG. 18 shows the nucleic acid sequence for the tobamovirus movement protein open reading frame of ToMV (SEQ ID NO: 10) as taught in Example 15.

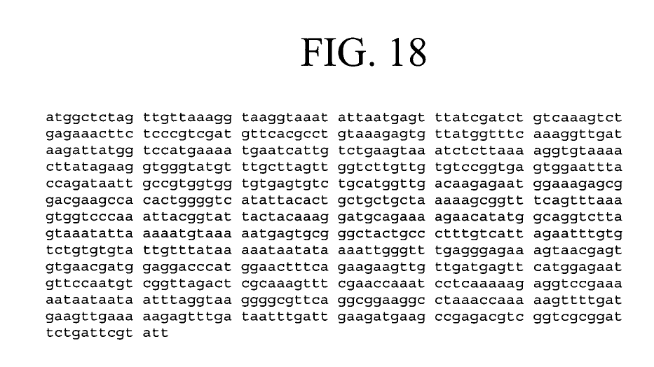

### DEFINITIONS

In order to provide a clear and consistent understanding of the specification and the claims, including the scope given herein to such terms, the following definitions are provided:

As used herein the term “amplification” refers to a process where the number of copies of a polynucleotide is increased. This may be done directly on the molecule or indirectly such as by placing it in a cell (e.g. by transformation, transinfection, etc.) and allowing the cell to naturally replicate the polynucleotide.

As used herein the term “annealing” refers to the formation of at least partially double stranded nucleic acid by hybridization of at least partially complementary nucleotide sequences. A partially double stranded nucleic acid can be due to the hybridization of a smaller nucleic acid strand to a longer nucleic acid strand, where the smaller nucleic acid is 100% identical to a portion of the larger nucleic acid. A partially double stranded nucleic acid can also be due to the hybridization of two nucleic acid strands that do not share 100% identity but have sufficient homology to hybridize under a particular set of hybridization conditions.

As used herein the term “clamp” refers to a unique nucleotide sequence added to one end of a polynucleotide, such as by incorporation of the clamp sequence into a PCR primer. The clamp sequences are intended to allow amplification only of polynucleotides that arise from hybridization of strands from different parents (i.e., heteroduplex molecules) thereby ensuring the production of full-length hybrid products as described previously (Skarfstad, J. Bact, vol 182, No 11, P.3008-3016).

As used herein the term “cleaving” means digesting the polynucleotide with enzymes or otherwise breaking phosphodiester bonds within the polynucleotide.

As used herein the term “complementary base pair” refers to the correspondence of DNA (or RNA) bases in the double helix such that adenine in one strand is opposite thymine (or uracil) in the other strand and cytosine in one strand is opposite guanine in the other.

As used herein the term “complementary to” is used herein to mean that the complementary sequence is identical to the reverse-complement of all or a portion of a reference polynucleotide sequence or that each nucleotide in one strand is able to form a base-pair with a nucleotide, or analog thereof in the opposite strand. For illustration, the nucleotide sequence “TATAC” is complementary to a reference sequence “GTATA”.

As used herein the term “denaturing” or “denatured,” when used in reference to nucleic acids, refers to the conversion of a double stranded nucleic acid to a single stranded nucleic acid. Methods of denaturing double stranded nucleic acids are well known to those skilled in the art, and include, for example, addition of agents that destabilize base-pairing, increasing temperature, decreasing salt, or combinations thereof. These factors are applied according to the complementarity of the strands, that is, whether the strands are 100% complementary or have one or more non-complementary nucleotides.

As used herein the term “desired functional property” means a phenotypic property, which include but are not limited to, encoding a polypeptide, promoting transcription of linked polynucleotides, binding a protein, improving the function or biological properties of a polypeptide, organism or vector, and the like, which can be selected or screened for. Polynucleotides with such desired functional properties, can be used in a number of ways, which include but are not limited to expression from a suitable plant, animal, fungal, yeast, or bacterial expression vector, integration to form a transgenic plant, animal or microorganism, expression of a ribozyme, and the like.

As used herein the term “DNA shuffling” is used herein to indicate reassortment of sequence information between substantially homologous but non-identical sequences.

As used herein, the term “effective amount” refers to the amount of an agent necessary for the agent to provide its desired activity. For the present invention, this determination is well within the knowledge of those of ordinary skill in the art.

As used herein the term “exonuclease” refers to an enzyme that cleaves nucleotides one at a time from an end of a polynucleotide chain, that is, an enzyme that hydrolyzes phosphodiester bonds from either the 3′ or 5′ terminus of a polynucleotide molecule. Such exonucleases, include but are not limited to T4 DNA polymerase, T7 DNA polymerase, E. coli Pol 1, and Pfu DNA polymerase. The term “exonuclease activity” refers to the activity associated with an exonuclease. An exonuclease that hydrolyzes in a 3′ to 5′ direction is said to have “3′ to 5′ exonuclease activity.” Similarly an exonuclease with 5′ to 3′ activity is said to have “5′ to 3′ exonuclease activity.” It is noted that some exonucleases are known to have both 3′ to 5′, 5′ to 3′ activity, such as, E. coli Pol I.

As used herein the term “Genetic Reassortment by Mismatch Resolution (GRAMMR)” refers to a method for reassorting sequence variations among related polynucleotides by an in vitro method of redistributing sequence variations between non-identical polynucleotide sequences, by making a heteroduplex polynucleotide from two non-identical polynucleotides; introducing a nick in one strand at or near a base pair mismatch site; removing mismatched base(s) from the mismatch site where the nick occurred; and using the opposite strand as template to replace the removed base(s) with bases that complement base(s) in the first strand. By this method, information is transferred from one strand to the other at sites of mismatch.

Multiple sites in a partially complementary molecule can be addressed independently and simultaneously in this procss. The result is an increase in the percentage of complementary base pairs in the polynucleotide sequence.

One or more base pair mismatches between two strands of the heteroduplex polynucleotide sequence are resolved by an in vitro method of mixing the heteroduplex polynucleotide sequence with an effective amount of mismatch-directed strand cleavage activity, proofreading activity, and ligase activity to resolve one or more of the mismatches. By this method, information is transferred from one strand to the other at sites of a mismatch.

A mismatch can be the result of two non-complementary bases occurring opposite each other. A mismatch site can consist of a cluster of any number of unpaired nucleotides, including nucleotide base-pairs that are made unstable by neighboring mismatches. A mismatch can also be the result of one or more bases occurring on one strand that do not have a numerical opposite on the opposite strand. For example, at the site of a mismatch there might be 1 unpaired base on one strand and no unpaired bases on the other strand. This would result in a site of sequence length heterogeneity in which a single unpaired nucleotide is contained in one strand at that site. Depending on the strand that is initially nicked at this site of mismatch, the process of this invention would result in either the insertion of a single base relative to the shorter strand, or in the deletion of a single base relative to the strand that originally had the extra unpaired nucleotide. This principle of transfer of sequence length information from one strand to the other can apply to any site of mismatch where the number of mismatched bases on the two strands do not equal one-another.

Usually many copies of the heteroduplex polynucleotide are present in the reaction. In this situation, sequence information at a mismatch site might be templated from the top strand on one copy of the polynucleotide and from the bottom strand in another copy. Assuming a sufficient number of copies are available, if a single mismatch is present, then two output variants are possible. If two mismatch sites are present then 2 times 2 variants can result. If n mismatch sites are present, then at least 2 to the n power or 2ⁿ genetic reassortments are possible by mismatch resolution. The possible result is at least 2ⁿ variant polynucleotides. We say at least, because the exact mechanism is not fully understood. It can be speculated that for a mismatch site that is 2 or more bases in length, an individual event might template 1, 2 or more of the mismatched bases. If that is the case, then the result would be an increase in the probable number of variants.

As used herein, the term “GENEWARE” or “GENEWARE®” refers to a viral vector derived at least in part from a Tobamovirus and modified to contain an additional (usually heterologous) subgenomic promoter. A Tobamovirus found in nature, typically contains subgenomic promoters for the movement protein and the coat protein. GENEWARE® is a registered trademark of Large Scale Biology corporation.

As used herein the term “granularity” refers to the amount of a nucleic acid's sequence information from a given parental polynucleotide sequence that occurs as a contiguous sequence in a given progeny polynucleotide.

As used herein the term “template sequence” refers to a first single stranded polynucleotide sequence that is partially complementary to a second polynucleotide sequence such that treatment by GRAMMR results in transfer of genetic information from the template strand to the second strand.

The larger the units of sequence information transferred from a template strand, the higher the granularity. The smaller the blocks of sequence information transferred from the template strand, the lower or finer the granularity. Lower granularity indicates that a DNA shuffling or reassortment method is able to transfer smaller discrete blocks of genetic information from the template strand to the second strand. The advantage of a DNA shuffling or reassortment method with lower granularity is that it is able to resolve smaller nucleic acid sequences from others, and to transfer the sequence information. DNA shuffling or reassortment methods that return primarily high granularity are not readily able to resolve smaller nucleic acid sequences from others.

As used herein the term “heteroduplex polynucleotide” refers to a double stranded polynucleotide formed by annealing single strands, typically separate strands, where the strands are non-identical. A heteroduplex polynucleotide may have unpaired regions existing as single strand loops or bubbles. A heteroduplex polynucleotide region can also be formed by one single-strand polynucleotide wherein partial self-complementarity allows the formation of a stem-loop structure where the annealing portion of the strand is non-identical.

As used herein the term “heteroduplex DNA” refers to a double-stranded DNA formed by annealing single strands, typically separate strands), where the strands are non-identical. A heteroduplex DNA may have unpaired regions existing as single strand loops or bubbles. A heteroduplex DNA region can also be formed by one single-strand polynucleotide wherein partial self-complementarity allows the formation of a stem-loop structure where the annealing portion of the strand is non-identical.

As used herein the term “homologous” means that one single-stranded nucleic acid sequence may hybridize to an at least partially complementary single-stranded nucleic acid sequence. The degree of hybridization may depend on a number of factors including the amount of identity between the sequences and the hybridization conditions such as temperature and salt concentrations as discussed later.

Nucleic acids are “homologous” when they are derived, naturally or artificially, from a common ancestor sequence. During natural evolution, this occurs when two or more descendent sequences diverge from a parent sequence over time, i.e., due to mutation and natural selection. Under artificial conditions, divergence occurs, e.g., in one of two basic ways. First, a given sequence can be artificially recombined with another sequence, as occurs, e.g., during typical cloning, to produce a descendent nucleic acid, or a given sequence can be chemically modified, or otherwise manipulated to modify the resulting molecule. Alternatively, a nucleic acid can be synthesized de novo, by synthesizing a nucleic acid that varies in sequence from a selected parental nucleic acid sequence. When there is no explicit knowledge about the ancestry of two nucleic acids, homology is typically inferred by sequence comparison between two sequences. Where two nucleic acid sequences show sequence similarity over a significant portion of each of the nucleic acids, it is inferred that the two nucleic acids share a common ancestor. The precise level of sequence similarity that establishes homology varies in the art depending on a variety of factors.

For purposes of this disclosure, two nucleic acids are considered homologous where they share sufficient sequence identity to allow GRAMMR-mediated information transfer to occur between the two nucleic acid molecules.

As used herein the term “identical” or “identity” means that two nucleic acid sequences have the same sequence or a complementary sequence. Thus, “areas of identity” means that regions or areas of a polynucleotide or the overall polynucleotide are identical or complementary to areas of another polynucleotide.

As used herein the term “increase in percent complementarity” means that the percentage of complementary base-pairs in a heteroduplex molecule is made greater.

As used herein the term, “ligase” refers to an enzyme that establishes a phosphodiester bond between adjacent nucleotides in a nucleic acid.

As used herein the term “mismatch” refers to a base-pair that is unable to form normal base-pairing interactions (i.e., other than “A” with “T” (or “U”), or “G” with “C”).

As used herein the term “mismatch resolution” refers to the conversion of a mismatched base-pair into a complementary base-pair.

As used herein the term “mutations” means changes in the sequence of a wild-type or reference nucleic acid sequence or changes in the sequence of a polypeptide. Such mutations can be point mutations such as transitions or transversions. The mutations can be deletions, insertions or duplications.

As used herein the term “nick translation” refers to the property of a polymerase where the combination of a 5′-to-3′ exonuclease activity with a 5′-to-3′ polymerase activity allows the location of a single-strand break in a double-stranded polynucleotide (a “nick”) to move in the 5′-to-3′ direction.

As used herein, the term “nucleic acid” or “nucleic acid molecule” means a polynucleotide such as deoxyribonucleic acid (DNA) or ribonucleic acid (RNA) and encompasses single-stranded and double-stranded nucleic acid as well as an oligonucleotide. Nucleic acids useful in the invention include genomic DNA, cDNA, mRNA, plasmids, cosmids, PCR products, and synthetic oligonucleotides, and can represent the sense strand, the anti-sense strand, or both. A nucleic acid generally incorporates the four naturally occurring nucleotides adenine, guanine, cytosine, and thymidine/uridine. An invention nucleic acid can also incorporate other naturally occurring or non-naturally occurring nucleotides, including derivatives thereof, so long as the nucleotide derivatives can be incorporated into a polynucleotide by a polymerase at an efficiency sufficient to generate a desired polynucleotide product.

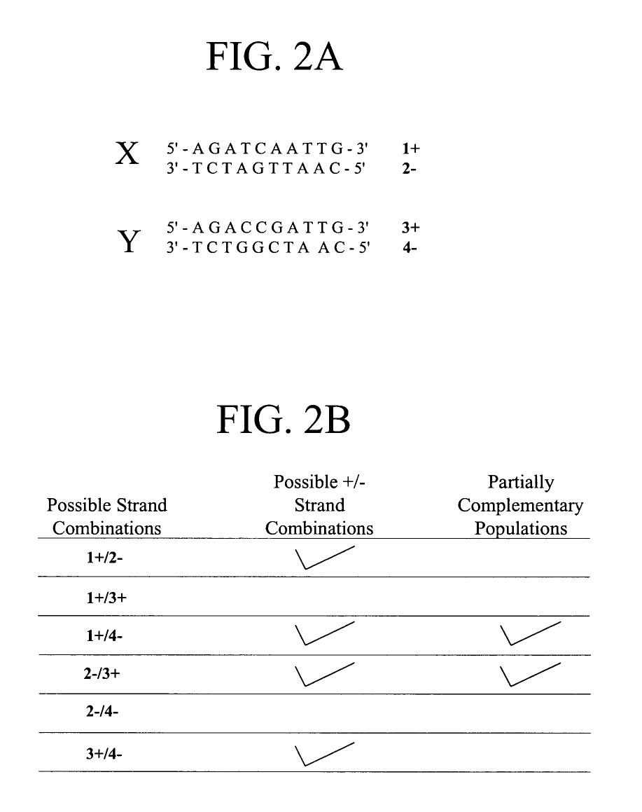

As used herein the term a “parental nucleic acid” refers to a nucleic acid having a sequence that is 100% identical to an original single stranded nucleic acid in a starting population of partially complementary nucleic acids. Parental nucleic acids would include, for example in the illustration of FIG. 2, nucleic acids X and Y if partially complementary nucleic acid combinations 1+/4− or 2−/3+ were used as a starting population in an invention method.

As used herein the term, “partially complementary” refers to a nucleic acid having a substantially complementary sequence to another nucleic acid but that differs from the other nucleic acid by at least two or more nucleotides.

As used herein the term, “partially complementary nucleic acid population” refers to a population of nucleic acids comprising individual groups of nucleic acids having substantially complementary sequences but no nucleic acids belonging to a particular group having an exact complementary sequence for any other group of sequences in the population.

As used herein, any member of a partially complementary nucleic acid population differs from another nucleic acid of the population, or the complement thereto, by two or more nucleotides. As such, a partially complementary nucleic acid specifically excludes a population containing sequences that are exactly complementary, that is, a complementary sequence that has 100% complementarity. Therefore, each member of such a partially complementary nucleic acid population differs from other members of the population by two or more nucleotides, including both strands. One strand is designated the top strand, and its complement is designated the bottom strand.

As used herein the term, “top” strand refers to a polynucleotide read in the 5′ to 3′ direction and the “bottom” its complement. It is understood that, while a sequence is referred to as bottom or top strand, such a designation is intended to distinguish complementary strands since, in solution, there is no orientation that fixes a strand as a top or bottom strand.

For example, a population containing two nucleic acid members can be derived from two double stranded nucleic acids, with a potential of using any of the four strands to generate a single stranded partially complementary nucleic acid population. An example of potential combinations of strands of two nucleic acids that can be used to obtain a partially complementary nucleic acid population of the invention is shown in FIG. 2. The two nucleic acid sequences that are potential members of a partially complementary nucleic acid population are designated “X” (AGATCAATTG) and “Y” (AGACCGATTG)(FIG. 2A). The nucleic acid sequences differ at two positions (positions 4 and 6indicated by “*”). The “top” strand of nucleic acids X and Y are designated “1+” and “3+,” respectively, and the “bottom” strand of nucleic acids X and Y are designated “2−” and “4−,” respectively.

FIG. 2B shows the possible combinations of the four nucleic acid strands. Of the six possible strand combinations, only the combination of 1+/2−, 1+/4−, 2−/3+, or 3+/4− comprise the required top and bottom strand of a partially complementary nucleic acid population. Of these top/bottom sequence combinations, only 1+/4− or 2−/3+ comprise an example of a partially complementary nucleic acid population of two different molecules because only these combinations have complementary sequences that differ by at least one nucleotide. The remaining combinations, 1+/2− and 2+/4−, contain exactly complementary sequences and therefore do not comprise a partially complementary nucleic acid population of the invention.

In the above described example of a population of two different molecules, a partially complementary population of nucleic acid molecules excluded combinations of strands that differ by one or more nucleotides but which are the same sense, for example, 1+/3+ or 2−/4−. However, it is understood that such a combination of same stranded nucleic acids can be included in a larger population, so long as the population contains at least one bottom strand and at least one top strand. For example, if a third nucleic acid “Z,” with strands 5+ and 6− is included, the combinations 1+/3+/6− or 2−/4−/5+ would comprise a partially complementary nucleic acid population. Similarly, any number of nucleic acids and their corresponding top and bottom strands can be combined to generate a partially complementary nucleic acid population of the invention so long as the population contains at least one top strand and at least one bottom strand and so long as the population contains no members that are the exact complement.

The populations of nucleic acids of the invention can be about 3 or more, about 4 or more, about 5 or more, about 6 or more, about 7 or more, about 8 or more, about 9 or more, about 10 or more, about 12 or more, about 15 or more, about 20 or more, about 25 or more about 30 or more, about 40 or more, about 50 or more, about 75 or more, about 100 or more, about 150 or more, about 200 or more, about 250 or more, about 300 or more, about 350 or more, about 400 or more, about 450 or more, about 500 or more, or even about 1000 or more different nucleic acid molecules. A population can also contain about 2000 or more, about 5000 or more, about 1×10⁴ or more, about 1×10⁵ or more, about 1×10⁶ or more, about 1×10⁷ or more, or even about 1×10⁸ or more different nucleic acids. One skilled in the art can readily determine a desirable population to include in invention methods depending on the nature of the desired reassortment experiment outcome and the available screening methods, as disclosed herein.

As used herein the term, a “polymerase” refers to an enzyme that catalyzes the formation of polymers of nucleotides, that is, polynucleotides in a template-directed fashion. A polymerase useful in the invention can be derived from any organism or source, including animal, plant, bacterial and viral polymerases. A polymerase can be a DNA polymerase, RNA polymerase, or a reverse transcriptase capable of transcribing RNA into DNA.

As used herein the term “proofreading” describes the property of an enzyme where a nucleotide, such as, a mismatched nucleotide, can be removed in a 3′-to-5′ fashion and replaced by, typically, a base-paired nucleotide. In the case of addressing a loop caused by insertion or deletion, proofreading may involve only removal of the mismatched nucleotide(s) or only addition of base-paired nucleotide(s).

As used herein the term, a “recombinant” polynucleotide refers to a polynucleotide that comprises sequence information from at least two different polynucleotides.

As used herein the term “related polynucleotides” means that regions or areas of the polynucleotides are identical and regions or areas of the polynucleotides are non-identical.

As used herein the term DNA “reassortment” is used herein to indicate a redistribution of sequence variations between non-identical sequences.

As used herein the term “replicon” refers to a genetic unit of replication including a length of polynucleotide and its site for initiation of replication.

As used herein the term “sequence diversity” refers to the abundance of non-identical polynucleotides. The term “increasing sequence diversity in a population” means to increase the relative abundance of non-identical polynucleotides in a population.

As used herein the term “sequence variant” refers to a molecule (DNA, RNA polypeptide, and the like) with one or more sequence differences compared to a reference molecule. For example, the sum of the separate independent mismatch resolution events that occur throughout the heteroduplex molecule during the GRAMMR process results in reassortment of sequence information throughout that molecule. The sequence information will reassort in a variety of combinations to generate a complex library of “sequence variants”.

As used herein the term “strand cleavage activity” or “cleavage” refers to the breaking of a phosphodiester bond in the backbone of the polynucleotide strand, for example forming a nick. Strand cleavage activity can be provided by an enzymatic agent. Such agents include, but are not limited to CEL I, RES I, T4 endonuclease VII, or T7 endonuclease I. S1 nuclease, BAL-31 nuclease, FEN1, cleavase, pancreatic DNase I, SP nuclease, mung bean nuclease, and nuclease P1; by a chemical agent, such agents include, but are not limited to potassium permanganate, tetraethylammonium acetate, sterically bulky photoactivatable DNA intercalators, [Rh(bpy)2(chrysi)]3+, osmium tetroxide with piperidine, and hydroxylamine with piperidine; or by energy in the form of ionizing radiation, or kinetic radiation.

As used herein the term “Mismatch-directed strand cleavage” means strand cleavage activity by an agent that recognizes a site of a mismatched base pair, group of mismatched base pairs, or extrahelical base or bases on a heteroduplex polynucleotide sequence and cleaves one strand at the site of the mismatch. While a small amount of double stranded cleavage and/or random single stranded cleavage may occur, the primary focus of the reaction is to nick at the site of a mismatched base pair.

As used herein the term “sufficient time” refers to the period of time necessary for a reaction or process to render a desired product. For the present invention, the determination of sufficient time is well within the knowledge of those of ordinary skill in the art. It is noted that “sufficient time” can vary widely, depending on the desires of the practitioner, without impacting on the functionality of the reaction, or the quality of the desired product.

As used herein the term “wild-type” means that a nucleic acid fragment does not contain any mutations. A “wild-type” protein means that the protein will be active at a level of activity found in nature and typically will be the amino acid sequence found in nature. In an aspect, the term “wild type” or “parental sequence” can indicate a starting or reference sequence prior to a manipulation of the invention.

In the polypeptide notation used herein, the left-hand direction is the amino terminal direction and the right-hand direction is the carboxy-terminal direction, in accordance with standard usage and convention. Similarly, unless specified otherwise, the left-hand end of single-stranded polynucleotide sequences is the 5′ end; the left-hand direction of double-stranded polynucleotide sequences is referred to as the 5′ direction. The direction of 5′ to 3′ addition of nascent RNA transcripts is referred to as the transcription direction.

### DETAILED DESCRIPTION OF THE INVENTION

The present invention provides an in vitro method of making sequence variants from at least one heteroduplex polynucleotide wherein the heteroduplex has at least two non-complementary nucleotide base pairs, the method comprising: preparing at least one heteroduplex polynucleotide; combining said heteroduplex polynucleotide with an effective amount of an agent or agents with strand cleavage activity proofreading activity, and ligase activity; and allowing sufficient time for the percentage of complementarity to increase, wherein at least one or more variants are made.

Another aspect of the present invention is where the heteroduplex polynucleotides are circular, linear or a replicon.

Another aspect of the present invention is where the desired variants have different amounts of complementarity.

Another aspect of the present invention is where the strand cleavage activity, proofreading activity, and ligase activity is added sequentially, or concurrently.

Another aspect of the present invention provides the addition of ligase activity, provided by agents such as, T4 DNA ligase, E. coli DNA ligase, or Taq DNA ligase.

In another aspect of the present invention, the strand cleavage activity is provided by an enzyme, such as, CEL I, RES I, T4 endonuclease VII, or T7 endonuclease I. S1 nuclease, BAL-31 nuclease, FEN1, cleavase, pancreatic DNase I, SP nuclease, mung bean nuclease, and nuclease P1; a chemical agent, such as, potassium permanganate, tetraethylammonium acetate, sterically bulky photoactivatable DNA intercalators, [Rh(bpy)2(chrysi)]3+, osmium tetroxide with piperidine, and hydroxylamine with piperidine or a form of energy, such as, ionizing or kinetic radiation.

In another aspect of the present invention, polymerase activity is provided by Pol beta.

Another aspect of the present invention is where both polymerase activity and 3′ to 5′ exonuclease activity is provided T4 DNA polymerase, T7 DNA polymerase, E. coli Pol 1, or Pfu DNA polymerase.

Another aspect of the present invention is where the agent with both polymerase activity and 5′ to 3′ exonuclease activity is E. coli Pol 1.

Another aspect of the present invention is where the agent with polymerase activity lacks 3′ to 5′ exonuclease activity (such as Taq DNA polymerase, VentR (exo-) DNA polymerase, Deep VentR (exo-) DNA polymerase, Therminator DNA polymerase, or Klenow Fragment (3′ to 5′exo-) (enzymes available from New England BioLabs), T4 DNA polymerase (3′ to 5′ exo-), or Klentaq (Barnes, Gene112(92)29), and the like), and the agent with the 3′ to 5′ exonuclease activity lacks polymerase activity (such as E. coli exonuclease III (Exo III) or Ape1 (Hadi, et al., J Mol Biol 316, (02)853)). In the case of polymerases with strand displacement activity, it is preferred to also add an agent with flap endonuclease activity such as T4 RNaseH (Bhagwat, et al., J Biol Chem 272 (1997) 28523) and the like.

In another aspect of the present invention, proofreading activity is provided T4 DNA polymerase or T7 DNA polymerase.

In another aspect of the present invention, the effective amount of strand cleavage activity, and proofreading activity and ligase activity are provided by RES I, T4 DNA polymerase, and E. coli DNA ligase.

In another aspect of the present invention, the effective amount of strand cleavage activity, and proofreading activity and ligase activity are provided by RES I, T7 DNA polymerase, and T4 DNA ligase.

Another embodiment of the present invention provides an in vitro method of increasing diversity in a population of sequences, comprising, preparing at least one heteroduplex polynucleotide; combining the heteroduplex polynucleotide with an effective amount of an agent or agents with proofreading activity, ligase activity and strand cleavage activity; and allowing sufficient time for the percentage of complementarity to increase, wherein diversity in the population is increased.

Another embodiment of the present invention provides a method of obtaining a polynucleotide encoding a desired functional property, comprising: preparing at least one heteroduplex polynucleotide; combining said heteroduplex polynucleotide with an effective amount of an agent or agents with proofreading activity, ligase activity and strand cleavage activity; allowing sufficient time for the percentage of complementarity between strands of the heteroduplex polynucleotide to increase, wherein diversity in the population is increased; and screening or selecting a population of variants for the desired functional property.

Another embodiment of the present invention provides a method of obtaining a polynucleotide encoding a desired functional property, comprising: preparing at least one heteroduplex polynucleotide; combining said heteroduplex polynucleotide with an effective amount of an agent or agents with proofreading activity, ligase activity and strand cleavage activity; allowing sufficient time for the percentage of complementarity between strands of the heteroduplex polynucleotide to increase, wherein diversity in the population is increased; converting DNA to RNA; and screening or selecting a population of ribonucleic acid variants for the desired functional property.

Yet another embodiment of the present invention provides a method of obtaining a polypeptide having a desired functional property, comprising: preparing at least one heteroduplex polynucleotide; combining said heteroduplex polynucleotide with an effective amount of an agent or agents with proofreading activity, ligase activity and strand cleavage activity; allowing sufficient time for the percentage of complementarity between strands of said heteroduplex polynucleotide to increase, converting said heteroduplex polynucleotide to RNA, and said RNA to a polypeptide; and screening or selecting a population of polypeptide variants for said desired functional property.

Still another embodiment of the present invention provides a method of obtaining a polynucleotide encoding a desired functional property, comprising: preparing at least one heteroduplex polynucleotide, where the heteroduplex is optionally, about 95%, 90%, 85%, 80%, 75%, 62%, 58% or 47% identical, and about 100 base pairs, 1000 base-pairs, 10,000 base-pairs, or 100,000 base-pairs or more in size; combining said heteroduplex polynucleotide with an effective amount of an agent or agents with proofreading activity, ligase activity and strand cleavage activity; allowing sufficient time for the percentage of complementarity between strands of the heteroduplex polynucleotide to increase, screening or selecting for a population of variants having a desired functional property; denaturing said population of variants to obtain single strand polynucleotides; annealing said single strand polynucleotides to form at least one second heteroduplex polynucleotide; combining said second heteroduplex polynucleotide with an effective amount of an agent or agents with proofreading activity, ligase activity and strand cleavage activity; and allowing sufficient time for the percentage of complementarity between strands of the heteroduplex polynucleotide to increase and optionally screening or selecting for a population of variants having a desired functional property. The second heteroduplex may be formed from the population of variants previously formed alone or with one or both single stranded parent polynucleotides or with an alternative single stranded polynucleotide.

While the two strands may have many mismatches, the heteroduplex may also concentrate the mismatches in a relatively small region, as small as 15-20 base pairs, preferably at least about 100 base pairs and maybe much more as listed above. For example, when the heteroduplex is inserted in a vector such as in POP-GRAMMR, the majority of the plasmid is completely complemetary with only this relatively small region primarily containing the mismatches. The percentage of identity mentioned above may apply only to the relatively small region in such a circumstance.

The present invention is directed to a method for generating an improved polynucleotide sequence or a population of improved polynucleotide sequences, typically in the form of amplified and/or cloned polynucleotides, whereby the improved polynucleotide sequence(s) possess at least one desired phenotypic characteristic (e.g., encodes a polypeptide, promotes transcription of linked polynucleotides, binds a protein, improves the function of a viral vector, and the like) which can be selected or screened for. Such desired polynucleotides can be used in a number of ways such as expression from a suitable plant, animal, fungal, yeast, or bacterial expression vector, integration to form a transgenic plant, animal or microorganism, expression of a ribozyme, and the like.

GRAMMR provides for resolution of mismatched base pairs on heteroduplex DNA strands in an in vitro reaction. This reaction begins with cleavage of one strand or the other at or near a mismatch followed by excision of mismatched bases from the cleaved strand and polymerization to fill in the resulting gap with nucleotides that are templated to the sequence of the other strand. The resulting nick can be sealed by ligation to rejoin the backbone. The sum of the separate independent mismatch resolution events that occur throughout the heteroduplex molecule will result in reassortment of sequence information throughout that molecule. The sequence information will reassort in a variety of combinations to generate a complex library of sequence variants.

In one embodiment of GRAMMR, a library of mutants is generated by any method known in the art such as mutagenic PCR, chemical mutagenesis, etc. followed by screening or selection for mutants with a desired property. The mutant DNAs are mixed, denatured to single strands, and allowed to anneal. Partially complementary strands that hybridize will have non-base-paired nucleotides at the sites of the mismatches. Treatment with CEL I (Oleykowski et al., 1998; Yang et al., 2000), or a similar mismatch-directed activity, such as RES I, will cause nicking of one or the other polynucleotide strand 3′ of each mismatch. (In addition, CEL I or RES I can nick 3′ of an insertion/deletion resulting in reassortment of insertions/deletions.) The presence of a polymerase containing a proofreading activity (e.g., T4 DNA Pol) will allow excision of the mismatch, and subsequent 5′-to-3′ polymerase activity will fill in the gap using the other strand as a template. A polymerase that lacks 5′-3′ exonuclease activity and strand-displacement activity will fill in the gap and will cease to polymerize when it reaches the 5′ end of DNA located at the original CEL I cleavage site, thus re-synthesizing only short patches of sequence. DNA ligase (e.g., T4 DNA ligase or E. coli DNA ligase) can then seal the nick by restoring the phosphate backbone of the repaired strand. This process can occur simultaneously at many sites and on either strand of a given heteroduplex DNA molecule. The result is a randomization of sequence differences among input strands to give a population of sequence variants that is more diverse than the population of starting sequences. These output polynucleotides can be cloned directly into a suitable vector, or they can be amplified by PCR before cloning. Alternatively, the reaction can be carried out on heteroduplex regions within the context of a double-stranded circular plasmid molecule or other suitable replicon that can be directly introduced into the appropriate host following the GRAMMR reaction. In another alternative, the output polynucleotides can be transcribed into RNA polynucleotides and used directly, for example, by inoculation of a plant viral vector onto a plant, such as in the instance of a viral vector transcription plasmid. The resulting clones are subjected to a selection or a screen for improvements in a desired property. The overall process can then be repeated one or more times with the selected clones in an attempt to obtain additional improvements.

If the output polynucleotides are cloned directly, there is the possibility of incompletely resolved molecules persisting that, upon replication in the cloning host, could lead to two different plasmids in the same cell. These plasmids could potentially give rise to mixed-plasmid colonies. If it is desired to avoid such a possibility, the output polynucleotide molecules can be grown in the host to allow replication/resolution, the polynucleotides isolated and retransformed into new host cells.

In another embodiment, when sequence input from more than two parents per molecule is desired, the above procedure is performed in a cyclic manner before any cloning of output polynucleotides. After the GRAMMR reaction, the double stranded polynucleotides are denatured, allowed to anneal, and the mismatch resolution process is repeated. After a desired number of such cycles, the output polynucleotides can be cloned directly, introduced into a suitable vector, or they can be amplified by PCR before cloning. The resulting clones are subjected to a selection or a screen for improvements in a desired property.

In another embodiment, a “molecular backcross” is performed to help eliminate the background of deleterious mutations from the desired mutations. A pool of desired mutant DNAs can be hybridized to wild-type DNA to perform the method. Clones can be selected for improvement, pooled, and crossed back to wild-type again until there is no further significant change.

The efficiency of the process is improved by various methods of enriching the starting population for heteroduplex molecules, thus reducing the number of unaltered parental-type output molecules. The mismatched hybrids can be affinity purified using aptamers, dyes, or other agents that bind to mismatched DNA. A preferred embodiment is the use of MutS protein affinity matrix (Wagner et al., Nucleic Acids Res. 23(19):3944-3948 (1995); Su et al., Proc. Natl. Acad. Sci. (U.S.A.), 83:5057-5061(1986)) or mismatch-binding but non-cleaving mutants of phage T4 endonuclease VII (Golz and Kemper, Nucleic Acids Research, 1999; 27: e7).

In one embodiment, the procedure is modified so that the input polynucleotides consist of a single strand of each sequence variant. For example, single-stranded DNAs of opposite strandedness are produced from the different parent sequences by asymmetric PCR to generate partially complementary single-stranded molecules. Annealing of the strands with one-another to make heteroduplex is performed as described in Example 1. Alternatively, single-stranded DNAs can be generated by preferentially digesting one strand of each parental double-stranded DNA with Lambda exonuclease followed by annealing the remaining strands to one-another. In this embodiment, the annealing strands have no 100% complementary strand present with which to re-anneal. Hence, there is a lower background of unmodified polynucleotides, that is, “parental polynucleotides” among the output polynucleotides leading to a higher efficiency of reassorting sequence variations. This increased efficiency will be particularly valuable in situations where a screen rather than a selection is employed to test for the desired polynucleotides.

Another method for heteroduplex formation is to mix the double-stranded parent DNAs, denature to dissociate the strands, and allow the single-stranded DNAs to anneal to one-another to generate a population of heteroduplexes and parental homoduplexes. The heteroduplexes can then be selectively enriched by a heteroduplex capture method such as those described above using MutS or a non-cleaving T4 endonuclease VII mutant. Alternatively, the parental homoduplex molecules in the population may be cleaved by restriction enzymes that overlap with sites of mismatch such that they are not cleaved in the heteroduplex but are cleaved in the parental homoduplex molecules. Uncleaved heteroduplex DNA can then be isolated by size fractionation in an agarose gel as was performed to generate full-length plasmid on full-length plasmid heteroduplex DNA molecules as describe in Example 5. Nick-sealing in those full-length heteroduplexed plasmid molecules was then brought about by incubation with DNA ligase.

In another embodiment, the parental, or input, double-stranded polynucleotides are modified by the addition of “clamp” sequences. One input polynucleotide or pool of polynucleotides is amplified by PCR with the addition of a unique sequence in the 5′ primer. The other input polynucleotide or pool is amplified by PCR with the addition of a unique sequence in the 3′ primer. The clamp sequences can be designed to contain a unique restriction enzyme site for the 5′ end of the gene of interest and another for the 3′ end such that, at the step of cloning the products of the GRAMMR reaction, only products with the 5′ clamp from the first polynucleotide (or pool) and the 3′ end from the second polynucleotide (or pool) will have appropriate ends for cloning. Alternatively, the products of the GRAMMR reaction can be PCR amplified using the unique sequences of the 5′ and 3′ clamps to achieve a similar result. Hence, there is a lower background of unmodified polynucleotides, that is, “parental polynucleotides” among the output polynucleotide clones leading to a higher efficiency of reassorting sequence variations. This increased efficiency will be particularly valuable in situations where a screen rather than a selection is employed to test for the desired polynucleotides. Optionally, oligonucleotide primers can be added to the GRAMMR reaction that are complementary to the clamp primer sequences such that either parent can serve as the top strand, thus permitting both reciprocal heteroduplexes to participate in the mismatch-resolution reaction.

Another method for generating cyclic heteroduplexed polynucleotides is performed where parental double-stranded DNAs have terminal clamp sequences as described above where the single-stranded clamp sequences extending from one end of the heteroduplex are complementary to single-stranded clamp sequences extending from the other end of the heteroduplex. These complementary, single-stranded clamps are allowed to anneal, thereby circularizing the heteroduplexed DNA molecule. Parental homoduplexes that result from re-annealing of identical sequences have only one clamp sequence and therefore, no complementary single-stranded sequences at their termini with which circularization can occur. Additionally, a DNA polymerase and a DNA ligase can be used to fill-in any gaps in the circular molecules and to seal the nicks in the backbone, respectively, to result in the formation of a population of covalently closed circular heteroduplex molecules. As the covalently-closed circular heteroduplex molecules will not dissociate into their component strands if subjected to further denaturating conditions, the process of denaturation, circularization, and ligation can be repeated to convert more of the linear double-stranded parental duplexes into closed into closed circular heteroduplexes.

In another embodiment, a region of a single-stranded circular vector such as a phagemid DNA can be hybridized to a related, but non-identical linear DNA, which can then be extended with a polymerase such as T7 DNA polymerase or T4 DNA polymerase plus T4 gene 32 protein, then ligated at the resulting nick to obtain a circular, double-stranded molecule with heteroduplexed regions at the sites of differences between the DNAs. GRAMMR can then be carried out on this molecule to obtain a library of sequence-reassorted molecules.

Alternately, two single-stranded circular vectors/phagemid DNAs of opposite strand polarity relative to the plasmid backbone, and parent gene sequences that are the target of the reassortment are annealed to one and other. A region of extensive mismatch will occur where the phage f1 origin sequences reside. Upon GRAMMR treatment, however, this region of extensive mismatch can revert to either parental type sequence restoring a functional f1 origin. These double-stranded molecules will also contain mismatch regions at the sites of differences between the strands encoding the parent genes of interest. GRAMMR can then be carried out on this molecule to obtain a library of sequence re-assorted molecule.

As discussed in the preceding paragraphs, the starting DNA or input DNA can be of any number of forms. For example, input DNA can be full-length, single stranded and of opposite sense, as is taught in Example 1. Alternatively, the input DNA can also be a fragment of the full-length strand. The input DNAs can be double-stranded, either one or both, or modified, such as by, methylation, phosphorothiolate linkages, peptide-nucleic acid, incorporation of uracil into the DNA, substitution of RNA in one or both strands, or the like. These modifications permit hybridization but may prevent amplification by various techniques such as PCR. If one does not wish for one strand to be amplifiable because it may not contain a variant sequence, that one strand may incorporate such a hybridizable but not amplifiable modification. Either strand of a duplex can be continuous along both strands, discontinuous but contiguous, discontinuous-with overlaps, or discontinuous with gaps.

GRAMMR can also be applied to DNA fragmentation and reassembly-based DNA shuffling schemes. For instance, in methods where gene fragments are taken through cycles of denaturation, annealing, and extension in the course of gene reassembly, GRAMMR can be employed as an intermediate step.

In one such embodiment, the DNA from a gene, or pool of mutant genes is fragmented by enzymatic, mechanical or chemical means, and optionally a size range of said fragments is isolated by a means such as separation on an agarose gel. The starting polynucleotide, such as a wild-type, or a desired variant, or a pool thereof, is added to the fragments and the mixture is denatured and then allowed to anneal. The annealed polynucleotides are treated with a polymerase to fill in the single stranded gaps using the intact strand as a template. The resulting partially complementary double strands will have non-base-paired nucleotides at the sites of the mismatches. Treatment with CEL I (Oleykowski et al., 1998; Yang et al., 2000), or an agent with similar activity, such as RES I, will cause nicking of one or the other polynucleotide strand 3′ of each mismatch. Addition of a polymerase containing a proofreading activity, such as T4 DNA Polymerase, will allow excision of the mismatch, and subsequent 5′-to-3′ polymerase activity will fill in the gap using the other strand as a template. A DNA ligase, such as, T4 DNA Ligase, can then seal the nick by restoring the phosphate backbone of the repaired strand. The result is a randomization of sequence variation among input strands to give output strands with potentially improved properties. These output polynucleotides can be cloned directly into a suitable vector, or they can be amplified by PCR before cloning. The resulting clones are subjected to a selection or a screen for improvements in a desired property.

In one such embodiment, the DNA from a pool of mutant genes is fragmented by enzymatic, mechanical or chemical means, or fragments are generated by limited extension of random oligonucleotides annealed to parental templates (U.S. Pat. No. 5,965,408), and optionally a size range of said fragments is isolated by a means such as separation on an agarose gel. The mixture is denatured and then allowed to anneal. The annealed polynucleotides are optionally treated with a polymerase to fill in the single stranded gaps. The resulting partially complementary double-strand fragments will have non-base paired nucleotides at the sites of the mismatches. Treatment with CEL I (Oleykowski et al., 1998; Yang et al., 2000), or an agent with similar activity, such as RES I, will cause nicking of one or the other polynucleotide strand 3′ of each mismatch. The activity of a polymerase containing a proofreading activity, such as T4 DNA Polymerase, will allow excision of the mismatch, and subsequent 5′-to-3′ polymerase activity will fill in the gap using the other strand as a template. Optionally, DNA ligase, such as, T4 DNA Ligase, can then seal the nick by restoring the phosphate backbone of the repaired strand. The result is a randomization of sequence variation among input strands to give output strands with potentially improved properties. Subsequent rounds of denaturing, annealing, and GRAMMR allows gene reassembly. PCR can be used to amplify the desired portion of the reassembled gene. These PCR output polynucleotides can be cloned into a suitable vector. The resulting clones are subjected to a selection or a screen for the desired functional property.

Another embodiment of the present invention provides starting with a continuous scaffold strand to which fragments of another gene or genes anneal. The flaps and gaps are trimmed and filled as is described in Coco, et al., Nature Biotech 19 (01)354; U.S. Pat. No. 6,319,713, and GRAMMR is performed. In this process, GRAMMR would bring about further sequence reassortment by permitting transfer of sequence information between the template strand and the strand resulting from flap and gap trimming and ligation. This method provides the benefits of incorporating specific sequence patches into one continuous strand followed by GRAMMR of residues that mismatch with the scaffold. By annealing many fragments simultaneously to the same sequence or gene, many individual sites can be addressed simultaneously, thereby allowing reassortment of multiple sequences or genes at once. In the present embodiment, the scaffold is not necessarily degraded, rather the duplex can be directly cloned, or amplified by PCR prior to cloning. Exhaustive mismatch resolution will result in a perfectly duplexed DNA. Partial mismatch resolution will result in essentially two different reassorted products per duplex.

As can be appreciated from the present disclosure, GRAMMR can also be applied to a variety of methods that include the annealing of related DNAs as a step in their process. For example, many site-directed mutagenesis protocols call for the annealing of mutant-encoding DNA molecules to a circular DNA in single-stranded form, either phagemid or denatured plasmid. These DNAs are then extended with a polymerase, followed by treatment with ligase to seal the nick, with further manipulation to remove the parental sequence, leaving the desired mutation or mutations incorporated into the parental genetic background. Though these protocols are generally used to incorporate specific mutations into a particular DNA sequence, it is feasible that the GRAMMR reaction can be applied to the heteroduplexed molecules generated in such a process to reassort sequence variations between the two strands, thereby resulting in a diverse set of progeny with reassorted genetic variation.

Another embodiment provides for sequential rounds of reassortment on only a particular region of the DNA of interest. For example, DNA fragments are annealed to a circular single-strand phagemid DNA, and GRAMMR is performed. The fragments can be treated in order to prevent them from being physically incorporated into the output material. For example, they can be terminated at the 3′ end with di-deoxy residues making them non-extendible. Multiple rounds of reassortment can be performed, but only modified molecules from the original input single stranded DNA clone will be recovered. The consequence will be that the DNA fragments used in this reassortment will contribute only sequence information to the final product and will not be physically integrated into the final recoverable product.

In instances where it is desired to resolve only sites of significant mismatch, that is patches of more than about 1 to 3 mismatches, S1 nuclease can be used. S1 nuclease is an endonuclease specific for single-stranded nucleic acids. It can recognize and cleave limited regions of mismatched base pairs in DNA:DNA or DNA:RNA duplexes. A mismatch of at least about 4 consecutive base pairs is generally required for recognition and cleavage by S1 nuclease. Mismatch resolution will not occur if both strands are cleaved, so the DNA must be repaired after the first nick and before the counter-nick. Other nucleases may be preferable for specifically tuning cleavage specificity according to sequence, sequence context, or size of mismatch.

In addition, other means of addressing mismatched residues, such as chemical-cleavage of mismatches may be used. Alternatively, one can choose to subject the strands of heteroduplexed DNA to random nicking with an activity such as that exhibited by DNaseI or an agent that cleaves only in duplexed regions. If nick formation occurs in a region of identity between the two genes, the DNA ligase present in the reaction will seal the nick with no net transfer of sequence information. However, if nick formation occurs near a site of mismatch, the mismatched bases can be removed by 3′-5′ exonuclease and the gap filled in by polymerase followed by nick sealing by ligase. Alternatively, application of nick-translation through regions of heterogeneity can bring about sequence reassortment. These processes, though not directed exclusively by the mismatch status of the DNA, will serve to transfer sequence information to the repaired strand, and thus result in a reassorted sequence.

GRAMMR can be used for protein, peptide, or aptamer display methods to obtain recombination between library members that have been selected. As fragmentation of the input DNAs is not required for GRAMMR, it may be possible to reassort sequence information between very small stretches of sequence. For instance, DNAs encoding small peptides or RNA aptamers that have been selected for a particular property such as target binding can be reassorted. For annealing to occur between the selected DNA molecules, some level of sequence homology should be shared between the molecules, such as at the 5′ and 3′ regions of the coding sequence, in regions of the randomized sequence segment that bear similarity because of similar binding activities, or through the biasing of codon wobble-base identity to a particular set of defaults. Complementary regions may be added to the 5′ and/or 3′ end to increase the annealing ability of strands with less homology, in order to aid in forming a heteroduplex

Manipulation of the reaction temperature at which GRAMMR is conducted can be useful. For example, lower temperatures will help to stabilize heteroduplexes allowing GRAMMR to be performed on more highly mismatched substrates. Likewise, additives that affect base-pairing between strands, such as salts, PEG, formamide, etc, can be used to alter the stability of the heteroduplex in the GRAMMR reaction, thereby affecting the outcome of the reaction.

In another embodiment, the mismatched double stranded polynucleotides are generated, treated with a DNA glycosylase to form an apurinic or apyrimidinic site, (that is an “AP site”) an AP endonuclease activity to cleave the phosphodiester bond, deoxyribulose phosphodiesterase to remove the deoxyribose-phosphate molecules, DNA polymerase β or other DNA polymerase to add a single nucleotide to the 3′ end of the DNA strand at the gap, and DNA ligase to seal the gap. The result is a reassortment of sequence variations between input strands to give output strands with potentially improved properties. These output polynucleotides can be cloned directly into a suitable vector, or they can be amplified by PCR before cloning. The resulting clones are subjected to a selection or a screen for improvements in a desired property.

Another embodiment provides for zonal mutagenesis by GRAMMR, that is, random or semi-random mutations at, and in the immediate vicinity of (generally within about 30 bases), mismatched residues using nucleotide analogues that have multiple base-pairing potential. This provides for concentration of essentially random mutagenesis at a particular point of interest, and adds another benefit to the present invention. Groups of genes that are similar, but have slightly different functions from one-another, for example, many enzymes, will exhibit moderate sequence differences from one-another in regions that will be operative for their own particular activities. These activities, can include substrate preference, binding partners, regulatory sites, or the like. Gene sequences that govern these functions should be heterogeneous within the population of related genes. Since it is known that the specificity of such function is associated with these amino acids and their neighbors, GRAMMR mutagenesis, in addition to reasserting sequence information between genes, may also be used to direct random mutagenesis to these regions to evolve their function, while not disturbing other sequences, such as structural framework, invariant residues, and other such important sites, that are potentially less tolerant to randomization.

Different enzymes with distinct functions will not differ just in the operative regions, such as active sites and regulatory sites. They are likely to have other differences from one another that arise through genetic drift. Further randomization in the locales of such changes might therefore be considered neutral, minimally important, or deleterious to the outcome of a mutagenesis experiment. In order to direct the random mutagenesis away from such inconsequential sites, and toward sites that might present a better result for random mutagenesis, such as the active site of an enzyme, the codon usage bias of the genes could be manipulated to decrease or increase the overall level of nucleotide complementarity in those regions. If regions of greater complementarity are less susceptible to GRAMMR than regions of lesser complementarity, then the degree of GRAMMER-directed zonal random mutagenesis at a given site can be modulated.

In another embodiment, after heteroduplex molecules are formed, an enzyme with a 3′ to 5′ exonuclease activity is added such that one strand of each end of the heteroduplex is digested back. At a point at which, on average, a desired amount of 3′ to 5′ digestion has occurred, dNTPs are added to allow the 5′ to 3′ polymerase activity from the same or an additional enzyme to restore the duplex using the opposite strand as a template. Thus mismatches in the digested regions are resolved to complementarity. Optionally, the resultant duplexes are purified, denatured and then allowed to anneal. The process of digestion, then polymerization is repeated resulting in new chimeric sequences. Additional cycles of the process can be performed as desired. Output duplex molecules are cloned and tested for the desired functional property. This process requires no fragmentation and reassembly. In addition, this process requires no endonucleolytic cleavages.

In another embodiment, after the heteroduplex molecules are formed, an enzyme with a 5′ to 3′ exonuclease activity, such as, T7 Gene6 Exonuclease as disclosed in Enger, M J and Richardson, C C, J Biol Chem 258(83)11197), is added such that one strand of each end of the heteroduplex is digested. At a point at which, on average, a desired amount of 5′ to 3′ digestion has occurred, the reaction is stopped and the exonuclease inactivated. Oligonucleotide primers complementary to the 5′ and 3′ ends of the target polynucleotides are added and annealed. A DNA polymerase, such as, T4 DNA Polymerase, a DNA ligase and dNTPs are added to allow the 5′ to 3′ polymerase activity to extend the primers and restore the duplex using the opposite strand as a template, with ligase sealing the nick. Thus mismatches in the digested regions are resolved to complementarity. Optionally, the resultant duplexes are purified, denatured and then allowed to anneal. The process of digestion then polymerization is repeated resulting in new chimeric sequences. Additional cycles of the process can be performed as desired. Output duplex molecules are cloned and tested for the desired functional property. This process requires no fragmentation and reassembly. In addition, this process requires no endonucleolytic cleavages.

In any DNA shuffling experiment, it is desirable to minimize the proportion of non-shuffled, or parental, DNAs that are obtained within the population of shuffled progeny. Numerous approaches may be used to accomplish this. In a plasmid-on-plasmid DNA shuffling format, where the genes to be shuffled are present on separate, but otherwise identical plasmids, each plasmid is linearized at one or another different unique restriction sites that are present. After removal of the restriction endonucleases, the linearized DNAs are mixed, melted apart, and allowed to anneal so that populations of heteroduplex DNA form that are either nicked, closed circular heteroduplex molecules, or are double stranded and linear homoduplexes. It is the population of circular double-stranded heteroduplex DNA molecules that represents the desired substrate for the GRAMMR reaction. One can either enrich this desired population by gel fractionation or use one or a number of methods that do not require physical separation of this population, but rather, discourages the recovery of non-shuffled parental molecules. Several such methods are listed below.

First, after GRAMMR reaction of the mixed population of linear parental homoduplex and circular double-stranded heteroduplex, transformation of E. coli is generally performed. Since circular DNA is vastly more efficient at transforming E. coli than its linearized counterpart, the parental homoduplexes can be strongly discriminated against at this step by preventing their circularization into transformation-competent molecules. The use of E. coli DNA ligase as the ligase component of the GRAMMR reaction will serve to prevent recircularization of parental homoduplex, as it more efficiently seals nicks than joins short cohesive termini that result from restriction endonuclease cleavage. Additionally, this enzyme very inefficiently ligates blunt ends. As a result of using this strategy, the progeny resulting from transformation of E. coli with the GRAMMR reaction are depleted of non-shuffled parental genes and enriched for molecules that entered the GRAMMR reaction as heteroduplex substrates.

Another method for excluding parental gene contamination from the population of GRAMMR output molecules is to position the plasmid linearization sites within a selectable marker. The sites should be of sufficient distance from one another to allow annealing to take place between staggered ends of a heteroduplex, and should either have overhangs that can be filled-in or trimmed off, or cause a deletion of sequence upon cleavage. As above, the plasmids containing the genes to be shuffled are linearized at one or other of the sites. After removal of the restriction endonucleases, the linearized DNAs are mixed, melted, and allowed to anneal. The resulting sample is made up of a mixture of circular heteroduplexes and of linear homoduplexes. This sample can then be treated with a proofreading polymerase such as T4 DNA polymerase in the presence of dNTPs. The circular homoduplexes should be unaffected, whereas the linear parental homoduplexes will have been blunted at their termini, effectively adding or deleting bases to the sequence of the selectable marker if that molecule becomes recircularized at any point in the GRAMMR reaction or after transformation into E. coli. If the addition or deletion of these sequences results in disruption of the function of the selectable marker, then the resulting molecules will not be recovered under appropriate selection.

Another method one can use to prevent unshuffled parental contamination of the shuffled library is to dephosphorylate the linearized DNAs prior to melting and annealing. Linear homoduplex molecules will be rendered unable to ligate into circular molecules whereas circular heteroduplexes will simply contain a single nick in each strand, but will still remain circular, and thus competent for efficient transformation into E. coli.

Another method one can use to prevent unshuffled parental contamination of the shuffled library is to digest with enzymes whose recognition sites are overlapped by mismatches in the heteroduplexed molecules. Digestion of the parental homoduplexes at those sites will render the resulting molecules linear so that they may be subject to any of the treatments described above to reduce parental contamination. The resulting molecules may also be made smaller, facilitating separation from the intact circular heteroduplex molecules.

If, in addition to excluding unshuffled parental molecules from a shuffling experiment, one desires to prevent shuffling between any two or more genes of a population of two or more parent genes, the same principles described above can be applied.

In the current invention the random reassortment occurs in an in vitro DNA mismatch-resolution reaction. This method does not require any steps of “gene reassembly” that serve as the foundation for the earlier mutation reassortment (“shuffling”) methods. Instead, it is based upon the ability of a reconstituted or artificial DNA mismatch resolving system to transmit sequence variations from one or more strands of DNA into another DNA strand by hybridization and mismatch resolution in vitro.

In general, standard techniques of recombinant DNA technology are described in various publications, e.g., (Ausubel, 1987; Ausubel, 1999; Sambrook et al., 1989), each of which is incorporated herein in their entirety by reference. Polynucleotide modifying enzymes were used according to the manufacturers recommendations. If desired, PCR amplimers for amplifying a predetermined DNA sequence may be chosen at the discretion of the practitioner.

It is noted that each of the activities taught in the present invention that are involved in the GRAMMR reaction can be interchanged with a functional equivalent agent with similar activity, and that such changes are within the scope of the present invention. For instance, as was indicated in Example 2, Taq DNA ligase could substitute for T4 DNA ligase. Other ligases can be substituted as well, such as E. coli DNA ligase. Likewise, as shown in Example 8, T7 DNA polymerase can be substituted for T4 DNA polymerase. Other enzymes with appropriate proofreading activity can function in place of any of these enzymes for the proofreading activity needed for the GRAMMR reaction. In a similar way, any polymerase with functionally equivalent activity to those demonstrated to work for GRAMMR can be used for substitution.

Strand cleavage may be brought about in a number of ways. In addition to CEL I, a number of functionally equivalent, and potentially similar activities found in extracts from a variety of plant species (Oleykowski, Nucleic Acids Res 1998;26:4597-602) may be used. Other mismatch-directed endonucleases such as T4 endonuclease VII, T7 endonuclease I, and SP nuclease (Oleykowski, Biochemistry 1999; 38: 2200-5) may be used. Another particularly useful mismatch-directed endonuclease is RES I. Other nucleases which attack single stranded DNA can be used, such as S1 nuclease, FEN1, cleavase, mung bean nuclease, and nuclease P1. Enzymes that make random cleavage events in DNA, such as pancreatic DNase I may also be substituted for the strand cleaving activity in GRAMMR. A number of methods for bringing about strand cleavage through other means are also envisioned. These include potassium permanganate used with tetraethylammonium acetate, the use of sterically bulky photoactivatable DNA intercalators such as [Rh(bpy)2(chrysi)]3+, osmium tetroxide with piperidine alkaloid, and hydroxylamine with piperidine alkaloid, as well as the use of radiation energy to bring about strand breakage.

Another embodiment to the present invention is directed to recombinant plant viral nucleic acids and recombinant viruses which are stable for maintenance and transcription or expression of non-native (foreign) nucleic acid sequences and which are capable of systemically transcribing or expressing such foreign sequences in the host plant. More specifically, recombinant plant viral nucleic acids according to the present invention comprise a native plant viral subgenomic promoter, at least one non-native plant viral subgenomic promoter, a plant viral coat protein coding sequence, and optionally, at least one non-native, nucleic acid sequence.

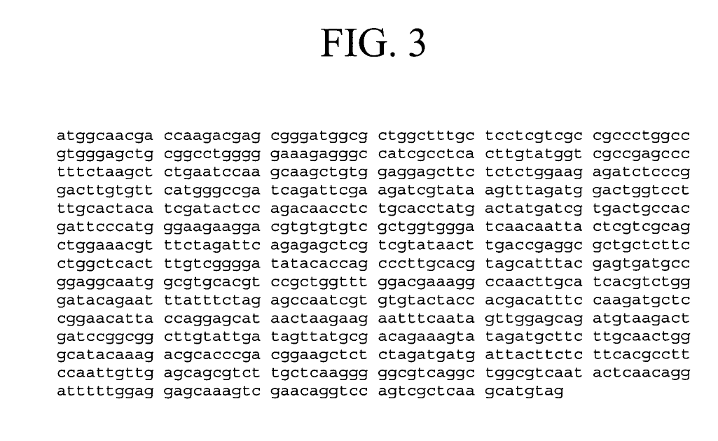

The present invention provides nucleic acid molecules comprising a nucleic acid sequence which include SEQ ID NO:01, SEQ ID NO:02, SEQ ID NO:03, or SEQ ID NO:04, useful as vectors or plasmids for the expression of CEL I endonuclease. The nucleic acid molecules of SEQ ID NO:03, and SEQ ID NO:04 are CEL I open reading frames contained within SEQ ID NO:01 and SEQ ID NO:02, respectively. The preparation and use of the nucleic acid molecules of SEQ ID NO:01, SEQ ID NO:02, SEQ ID NO:03 and SEQ ID NO:04, are further taught in Example 12 herein. The present invention also provides nucleic acid molecules comprising the nucleic acid sequence of FIG. 3 (SEQ ID NO: 16), useful as vectors or plasmids for the expression of RES I endonuclease.

The present invention further provides a plant cell comprising a vector or plasmid comprising of a nucleic acid sequence selected from the group consisting of SEQ ID NO:01, SEQ ID NO:02, SEQ ID NO:03, SEQ ID NO:04, or FIG. 3 (SEQ ID NO:16) where the plant cell is a host cell, or production cell.

The present invention also provides a recombinant plant viral nucleic acid comprising of at least one sub-genomic promoter capable of transcribing or expressing CEL I or RES I endonuclease in a plant cell, wherein the plant cell is a host cell, or production cell.

The present invention also provides a process for expressing CEL I or RES I endonuclease using a recombinant plant viral nucleic acid comprising of a nucleic acid sequence selected from the group consisting of SEQ ID NO:01, SEQ ID NO:02, SEQ ID NO:03, SEQ ID NO:04, or FIG. 3 (SEQ ID NO:16).

As used herein, the term “host” refers to a cell, tissue or organism capable of replicating a vector or plant viral nucleic acid and which is capable of being infected by a virus containing the viral vector or plant viral nucleic acid. This term is intended to include prokaryotic and eukaryotic cells, organs, tissues or organisms, where appropriate.

As used herein, the term “phenotypic trait” refers to an observable property resulting from the expression of a gene.

As used herein, the term “plant cell” refers to the structural and physiological unit of plants, consisting of a protoplast and the cell wall.

As used herein, the term “plant organ” refers to a distinct and visibly differentiated part of a plant, such as root, stem, leaf or embryo.

As used herein, the term “plant tissue” refers to any tissue of a plant in planta or in culture. This term is intended to include a whole plant, plant cell, plant organ, protoplast, cell culture, or any group of plant cells organized into a structural and functional unit.

As used herein, the term “production cell” refers to a cell, tissue or organism capable of replicating a vector or a viral vector, but which is not necessarily a host to the virus. This term is intended to include prokaryotic and eukaryotic cells, organs, tissues or organisms, such as bacteria, yeast, fungus and plant tissue.

As used herein, the term “promoter” refers to the 5′-flanking, non-coding sequence adjacent a coding sequence which is involved in the initiation of transcription of the coding sequence.

As used herein, the term “protoplast” refers to an isolated plant cell without cell walls, having the potency for regeneration into cell culture or a whole plant.

As used herein, the term “recombinant plant viral nucleic acid” refers to plant viral nucleic acid, which has been modified to contain non-native nucleic acid sequences.

As used herein, the term “recombinant plant virus” refers to a plant virus containing the recombinant plant viral nucleic acid.

As used herein, the term “substantial sequence homology” refers to nucleotide sequences that are substantially functionally equivalent to one another. Nucleotide differences between such sequences having substantial sequence homology will be de minimus in affecting function of the gene products or an RNA coded for by such sequence.

As used herein, the term “transcription” refers to production of an RNA molecule by RNA polymerase as a complementary copy of a DNA sequence.

As used herein, the term “vector” refers to a self-replicating DNA molecule which transfers a DNA segment between cells.

Useful phenotypic traits in plant cells include, but are not limited to, improved tolerance to herbicides, improved tolerance to extremes of heat or cold, drought, salinity or osmotic stress; improved resistance to pests (insects, nematodes or arachnids) or diseases (fungal, bacterial or viral) production of enzymes or secondary metabolites; male or female sterility; dwarfness; early maturity; improved yield, vigor, heterosis, nutritional qualities, flavor or processing properties, and the like. Other examples include the production of important proteins or other products for commercial use, such as lipase, melanin, pigments, antibodies, hormones, pharmaceuticals, antibiotics and the like. Another useful phenotypic trait is the production of degradative or inhibitory enzymes, such as are utilized to prevent or inhibit root development in malting barley. The phenotypic trait may also be a secondary metabolite whose production is desired in a bioreactor.

A still further feature of the invention is a process for the production of a specified polypeptide or protein product such as, but are not limited to, enzymes, complex biomolecules, a ribozyme, or polypeptide or protein products resulting from anti-sense RNA. Such products include, but are not limited to: IL-1, IL-2, IL-3, IL-4, IL-5, IL-6, IL-7, IL-8, IL-9, IL-10, IL-11, IL-12, etc.; EPO; CSF including G-CSF, GM-CSF, hPG-CSF, M-CSF, etc; Factor VIII; Factor IX; tPA; hGH; receptors and receptor antagonists; antibodies; neuro-polypeptides; melanin; insulin; vaccines and the like. The non-native nucleic acid of the RPVNA comprises the transcribable sequence, which leads to the production of the desired product. This process involves the infection of the appropriate plant host with a recombinant virus or recombinant plant viral nucleic acid such as those described above, the growth of the infected host to produce the desired product, and the isolation of the desired product, if necessary. The growth of the infected host is in accordance with conventional techniques, as is the isolation of the resultant product.

CEL I is a Mismatch Endonuclease

CEL I is a mismatch endonuclease isolated from celery. The use of CEL I in a diagnostic method for the detection of mutations in targeted polynucleotide sequences, in particular, those associated with cancer, is disclosed in U.S. Pat. No. 5,869,245. Methods of isolating and preparing CEL I are also disclosed in this patent. However, there is no disclosure in this patent relating to the use of CEL I in DNA sequence reassortment.

Nucleic acid molecules that encode CEL I are disclosed in PCT Application Publication No. WO 01/62974 A1. As with U.S. Pat. No. 5,869,245, the use of CEL I in a diagnostic method for the detection of mutations in targeted polynucleotide sequences associated with cancer is disclosed. Also similarly, there is no disclosure relating to the use of CEL I in DNA sequence reassortment.

RES I is a Mismatch Endonuclease

The use of RES I mismatch endonuclease is contemplated in diagnostic methods for the detection of mutations in targeted polynucleotide sequences, in particular, those associated with cancer. Examples of some of these types of diagnostic methods are disclosed in U.S. Pat. No. 5,869,245, Sokurenko, et al., and Del Tito, et al..

The reactivity of Endonuclease VII of phage T4 with DNA-loops of eight, four, or one nucleotide, or any of 8 possible base mismatches in vitro is disclosed in “Endonuclease VII of Phage T4 Triggers Mismatch Correction in Vitro” Solaro, et al., J Mol Biol 230(93)868. The publication reports a mechanism where Endonuclease VII introduces double stranded breaks by creating nicks and counternicks within six nucleotides 3′ of the mispairing. The publication discloses that a time delay between the occurrence of the first nick and the counternick was sufficient to allow the 3′-5′ exonuclease activity of gp43 to remove the mispairing and its polymerase activity to fill in the gap before the occurrence of the counternick. Nucleotides are erased from the first nick, which is located 3′ of the mismatch on either strand and stops 5′ of the mismatch at the first stable base-pair. The polymerase activity proceeds in the 5′ to 3′ direction towards the initial nick, which is sealed by DNA ligase. As a result, very short repair tracks of 3 to 4 nucleotides extend across the site of the former mismatch. The publication concludes with a discussion regarding the various activities Endonuclease VII may have within phage T4. However, the publication does not disclose any practical utility for Endonuclease VII outside of phage T4, and there is no disclosure regarding its applicability in DNA reassortment.

A method for creating libraries of chimeric DNA sequences in vivo in Escherichia coli is disclosed in Nucleic Acids Research, 1999, Vol 27, No. 18, e18, Volkov, A. A., Shao, Z., and Arnold, F. H. The method uses a heteroduplex formed in vitro to transform E. coli where repair of regions of non-identity in the heteroduplex creates a library of new, recombined sequences composed of elements of each parent. Although the publication discloses the use of this method as a convenient addition to existing DNA recombination methods, that is, DNA shuffling, the disclosed method is limited to the in vivo environment of E. coli. The publication states that there is more than one mechanism available for mismatch repair in E. coli, and that the ‘long patch’ repair mechanism, which utilizes the MutS/L/H enzyme system, was probably responsible for the heteroduplex repair.

The following non-limiting examples are provided to illustrate the present invention.

### CITED REFERENCES

1. Arkin, A. P. and Youvan, D. C. (1992) An algorithm for protein engineering: simulations of recursive ensemble mutagenesis. Proc Natl Acad Sci USA, 89, 7811-7815.

2. Ausubel, F. M. (1987) Current protocols in molecular biology. Published by Greene Pub. Associates and Wiley-Interscience: J. Wiley, New York.

3. Ausubel, F. M. (1999) Short protocols in molecular biology: a compendium of methods from Current protocols in molecular biology. Wiley, New York.

4. Barnes, W. M. (1994) PCR amplification of up to 35-kb DNA with high fidelity and high yield from lambda bacteriophage templates. Proc Natl Acad Sci USA, 91, 2216-2220.

5. Bartel, D. P. and Szostak, J. W. (1993) Isolation of new ribozymes from a large pool of random sequences. Science, 261, 1411-1418.

6. Cadwell, R. C. and Joyce, G. F. (1992) Randomization of genes by PCR mutagenesis. PCR Methods Appl, 2, 28-33.

7. Calogero, S., Bianchi, M. E. and Galizzi, A. (1992) In vivo recombination and the production of hybrid genes. FEMS Microbiol Lett, 76, 41-44.

8. Caren, R., Morkeberg, R. and Khosla, C. (1994) Efficient sampling of protein sequence space for multiple mutants. Biotechnology (N Y), 12, 517-520.

9. Delagrave, S., Goldman, E. R. and Youvan, D. C. (1993) Recursive ensemble mutagenesis. Protein Eng, 6, 327-331.

10. Delagrave, S. and Youvan, D. C. (1993) Searching sequence space to engineer proteins: exponential ensemble mutagenesis. Biotechnology (N Y), 11, 1548-1552.

11. Goldman, E. R. and Youvan, D. C. (1992) An algorithmically optimized combinatorial library screened by digital imaging spectroscopy. Biotechnology (N Y), 10, 1557-1561.

12. Gram, H., Marconi, L. A., Barbas, C. F. d., Collet, T. A., Lerner, R. A. and Kang, A. S. (1992) In vitro selection and affinity maturation of antibodies from a naive combinatorial immunoglobulin library. Proc Natl Acad Sci USA, 89, 3576-3580.

13. Hayashi, N., Welschof, M., Zewe, M., Braunagel, M., Dubel, S., Breitling, F. and Little, M. (1994) Simultaneous mutagenesis of antibody CDR regions by overlap extension and PCR. Biotechniques, 17, 310, 312, 314-315.

14. Hermes, J. D., Blacklow, S. C. and Knowles, J. R. (1990) Searching sequence space by definably random mutagenesis: improving the catalytic potency of an enzyme. Proc Natl Acad Sci USA, 87, 696-700.

15. Holland, J. H. (1992) Adaptation in natural and artificial systems: an introductory analysis with applications to biology, control, and artificial intelligence. MIT Press, Cambridge, Mass.

16. Ji, G. and Silver, S. (1992) Regulation and expression of the arsenic resistance operon from Staphylococcus aureus plasmid pI258. J Bacteriol, 174, 3684-3694.

17. Kauffman, S. A. (1993) The origins of order: self-organization and selection in evolution. Oxford University Press, New York.

18. Marton, A., Delbecchi, L. and Bourgaux, P. (1991) DNA nicking favors PCR recombination. Nucleic Acids Res, 19, 2423-2426.

19. Meyerhans, A., Vartanian, J. P. and Wain-Hobson, S. (1990) DNA recombination during PCR. Nucleic Acids Res, 18, 1687-1691.

20. Nissim, A., Hoogenboom, H. R., Tomlinson, I. M., Flynn, G., Midgley, C., Lane, D. and Winter, G. (1994) Antibody fragments from a ‘single pot’ phage display library as immunochemical reagents. EMBO J, 13, 692-698.

21. Oleykowski, C. A., Bronson Mullins, C. R., Godwin, A. K. and Yeung, A. T. (1998) Mutation detection using a novel plant endonuclease. Nucleic Acids Res, 26, 4597-4602.

22. Oliphant, A. R., Nussbaum, A. L. and Struhl, K. (1986) Cloning of random-sequence oligodeoxynucleotides. Gene, 44, 177-183.

23. Sambrook, J., Maniatis, T. and Fritsch, E. F. (1989) Molecular cloning: a laboratory manual. Cold Spring Harbor Laboratory, Cold Spring Harbor, N.Y.

24. Stemmer, W. P. (1994a) DNA shuffling by random fragmentation and reassembly: in vitro recombination for molecular evolution. Proc Natl Acad Sci USA, 91, 10747-10751.

25. Stemmer, W. P. (1994b) Rapid evolution of a protein in vitro by DNA shuffling. Nature, 370, 389-391.

26. Stemmer, W. P., Morris, S. K. and Wilson, B. S. (1993) Selection of an active single chain Fv antibody from a protein linker library prepared by enzymatic inverse PCR. Biotechniques, 14, 256-265.

27. Winter, G., Griffiths, A. D., Hawkins, R. E. and Hoogenboom, H. R. (1994) Making antibodies by phage display technology. Annu Rev Immunol, 12, 433-455.

28. Yang, B., Wen, X., Kodali, N. S., Oleykowski, C. A., Miller, C. G., Kulinski, J., Besack, D., Yeung, J. A., Kowalski, D. and Yeung, A. T. (2000) Purification, cloning, and characterization of the CEL I nuclease. Biochemistry, 39, 3533-3541.

29. Sokurenko, E. V., Tchesnokova, V., Yeung, A. T., Oleykowski, C. A., Trintchina, E., Hughes, K. T., Rashid, R. A., Brint, J. M., Moseley, S. L., Lory, S. (2001) Detection of simple mutations and polymorphisms in large genomic regions. Nucleic Acids Res, 29, e111.

30. Yang, T. T., Sinai, P., Green, G., Kitts, P. A., Chen, Y. T., Lybarger, L., Chervenak, R., Patterson, G. H., Piston, D. W., Kain, S. R. (1998) Improved fluorescence and dual color detection with enhanced blue and green variants of the green fluorescent protein. J Biol Chem 273, 8212-8216

31. Crameri, A., Whitehorn, E. A., Tate, E., Stemmer, W. P. (1996) Improved green fluorescent protein by molecular evolution using DNA shuffling. Nat Biotechnol 14, 315-319.

32. Heim, R., Prasher, D. C., Tsien, R. Y. (1994) Wavelength mutations and posttranslational autoxidation of green fluorescent protein. Proc Natl Acad Sci USA 91, 12501-12504.

33. Del Tito, B. J., Jr., Poff, H. E., 3^{rd}, Novotny, M. A., Cartledge, D. M., Walker, R. I., 2^{nd}, Earl, C. D., Bailey, A. L. (1998) Automated fluorescent analysis procedure for enzymatic mutation detection. Clin Chem 44, 731-739.

34. Barnes, W. M.: The fidelity of Taq polymerase catalyzing PCR is improved by an N-terminal deletion. Gene 112 (1992) 29-35.

35. Bhagwat, M., Hobbs, L. J. and Nossal, N. G.: The 5′-exonuclease activity of bacteriophage T4 RNase H is stimulated by the T4 gene 32 single-stranded DNA-binding protein, but its flap endonuclease is inhibited. J Biol Chem 272 (1997) 28523-30.

36. Hadi, M. Z., Ginalski, K., Nguyen, L. H. and Wilson, D. M., 3rd: Determinants in nuclease specificity of Ape1 and Ape2, human homologues of Escherichia coli exonuclease III. J Mol Biol 316 (2002) 853-66.

### EXAMPLE 1

### Cleavage of Mismatched DNA Substrate by CEL I

This example teaches the preparation of CEL I enzyme and its use in the cleavage of mismatched DNA substrate.

CEL I enzyme was prepared from celery stalks using the homogenization, ammonium sulfate, and Concanavalin A-Sepharose protocol described by Yang et al. (Biochemistry, 39:3533-3541 (2000), incorporated herein by reference. A 1.5 kg sample of chilled celery stalks was homogenized with a juice extractor. One liter of juice was collected, adjusted to 100 mM Tris-HCL, pH 7.7 with 100 micromolar phenylmethylsulfonyl fluoride (PMSF), and filtered through two layers of miracloth. Solid (NH₄)₂SO₄ was slowly added to 25% saturation while stirring on ice. After 30 minutes, the suspension was centrifuged at 27,000 g for 1.5 hours at 4° C. The supernatants were collected and adjusted with solid (NH₄)₂SO₄ to 80% saturation while stirring on ice followed by centrifugation at 27,000 g for 2 hours. The pellets were re-suspended in buffer B (0.1 M Tris-HCL, pH 7.7, 0.5 M KCl, 100 micromolar PMSF) and dialyzed against the same buffer.

Conconavalin A (ConA) Sepharose affinity chromatography was performed by first incubating the dialyzed sample with 2 ml of ConA resin overnight with gentle agitation. The ConA resin was then packed into a 0.5 cm diameter column and washed with several column volumes of buffer B. Elution was performed using 0.3 M alpha-methyl-mannoside in buffer B. Fractions were collected in 1 ml aliquots. Fractions were assayed for mismatch cleavage activity on a radiolabeled mismatch substrate by incubating 0.1 microliter of each fraction with the mismatched probe in buffer D (20 mM Tris-HCL, pH 7.4, 25 mM KCL, 10 mM MgCl₂) for 30 minutes at 45° C. as described by Oleykowski et al. (Nucleic Acids Research 26: 4597-4602 (1998), incorporated herein by reference. Reaction products were visualized by separation on 10% TBE-PAGE gels containing 7% urea (Invitrogen), followed by autoradiography. Aliquots of the CEL I fractions having mismatch cleavage activity were stored frozen at −20° C. A series of five-fold dilutions of CEL I fraction #5 were then analyzed for mismatch cleavage of radiolabeled mismatch substrate. Reactions were performed either in buffer D, New England BioLabs (NEB) T4 DNA ligase buffer (50 mM Tris-HCL, pH 7.5, 10 mM MgCl₂, 10 mM dithiothreitol (DTT), 1 mM ATP, 25 microgram/ml BSA), or Gibco/BRL T4 DNA ligase buffer (50 mM Tris-HCL, pH 7.6, 10 mM MgCl₂, 1 mM DTT, 1 mM ATP, 5%(w/v) polyethylene glycol-8000). Reaction products were visualized as above. Cleavage activity in buffer D and in NEB T4 DNA ligase buffer were found to be roughly equivalent, whereas cleavage in the PEG-containing Gibco/BRL ligase buffer was enhanced by five to ten-fold compared to the other buffers.

Additional analysis of CEL I activity was carried out using defined heteroduplex DNAs from two different Green Fluorescent Protein (GFP) genes as substrate. This GFP heteroduplex substrate was prepared by annealing single stranded DNAs corresponding to cycle 3 GFP (SEQ ID NO: 30) on the sense strand and wild-type GFP (SEQ ID NO: 29) on the antisense strand. The single-stranded DNAs had been synthesized by asymmetric PCR and isolated by agarose gel electrophoresis. After annealing by heating to 90° C. and cooling to room-temperature in the presence of 1×NEB restriction enzyme buffer 2 (10 mM Tris-HCL, pH 7.9, 10 mM MgCl₂, 50 mM NaCl, 1 mM dithiothreitol), the heteroduplex DNA was isolated by agarose gel electrophoresis followed by excision of the heterduplex band and extraction using Qiaquick DNA spin columns. A total of twenty eight mismatches, one or two nucleotides in length, occur throughout the length of the heteroduplex molecule. The distribution of the mismatches ranges from small clusters of several mismatches separated by one or two nucleotides to mismatches separated by more than thirty base pairs on either side.

A series of three-fold dilutions of CEL I in 1×NEB T4 DNA ligase buffer were prepared and one microliter aliquots of each were incubated in two separate series of 10 microliter reactions, each containing as substrate either 0.5 microgram of a supercoiled plasmid preparation or one hundred nanograms of the cycle3/wild-type GFP heteroduplex. All reactions took place in 1×NEB T4 DNA ligase buffer. Reactions were incubated at 45° C. for 30 minutes and run on 1.5% TBE-agarose gel in the presence of ethidium bromide.

Treatment of the supercoiled plasmid preparation with increasing amounts of CEL I resulted in the conversion of supercoiled DNA to nicked circular, then linear molecules, and then to smaller fragments of DNA of random size. Treatment of the mismatched GFP substrate with the CEL I preparation resulted in the digestion of the full-length heteroduplex into laddered DNA bands which are likely to represent cleavage on opposite DNA strands in the vicinity of clusters of mismatches. Further digestion resulted in the conversion of the mismatched GFP substrate to smaller DNAs that may represent a limit digest of the heteroduplex DNA by the CEL I preparation.

### EXAMPLE 2

### Conservation of Full Length GFP Gene with Mismatch Resolution Cocktails

This example teaches various mismatch resolution cocktails that conserve the full length GFP Gene.

Mismatched GFP substrate was treated with various concentrations of CEL I in the presence of cocktails of enzymes that together constitute a synthetic mismatch resolution system. The enzymes used were CEL I, T4 DNA polymerase, Taq DNA polymerase and T4 DNA ligase. CEL I activity should nick the heteroduplex 3′ of mismatched bases. T4 DNA polymerase contains 3′-5′ proofreading activity for excision of the mismatched base from the nicked heteroduplex. T4 DNA polymerase and Taq DNA polymerase contain DNA polymerase capable of filling the gap. T4 DNA ligase seals the nick in the repaired molecule. Taq DNA polymerase also has 5′ flap-ase activity.

Matrix experiments were performed to identify the reaction conditions that would serve to resolve mismatches in the GFP heteroduplex substrate. In one experiment, cycle 3/wild-type GFP heteroduplex was incubated in a matrix format with serial dilutions of CEL I fraction number five (described above) at eight different concentrations. Each reaction contained 100 nanograms of heteroduplex substrate and 0.2 microliters of T4 DNA ligase (Gibco BRL) in 1×NEBT4 DNA ligase buffer and dNTPs at 250 micromolar each, in a reaction volume of 10 microliters. In all, the matrix contained 96 individual reactions. One full set of reactions was incubated at room temperature for 30 minutes while another full set was incubated at 37° C. for 30 minutes.

After incubation, PCR was used to amplify the GFP gene from each reaction. Aliquots from each PCR were then digested with HindIII and HpaI and electrophoresed on 3% agarose gels with ethidium bromide. Only cycle 3 GFP has a HindIII site and only wild-type encodes a HpaI site.

If DNA mismatch resolution occurred at either the HindIII or HpaI mismatched sites, then a proportion of the PCR product would be expected to contain both sites, yielding a novel band. The band was observed in all samples, including the negative control samples that had neither CEL I, nor T4 DNA polymerase, nor Taq DNA polymerase. The results suggested that a basal level of background recombination may have occurred at some point in the experiment other than in the GRAMMR reaction; possibly in the PCR step. PCR-mediated recombination is known to occur at some frequency between related sequences during amplification Paabo, et al., J Biol Chem 265(90)4718-4721.

In another experiment, 200 nanograms of cycle 3/wild-type GFP heteroduplex was treated with CEL I and T4 DNA polymerase in various concentrations along with 2.5 units of Taq DNA polymerase in the presence or absence of T4 DNA ligase (0.2 units; Gibco BRL). Each reaction contained 1×NEB T4 DNA ligase buffer with 0.05 mM each dNTP in a final volume of 20 microliters. Reactions were incubated for 30 minutes at 37° C. and 10 microliters were run on a 2% TBE-agarose gel in the presence of ethidium bromide. Results showed that in the presence of DNA ligase, but in the absence of T4 DNA polymerase, increasing amounts of CEL I caused greater degradation of the heteroduplexed DNA, but that this effect could be counteracted by increasing the amount of T4 DNA polymerase in the reaction. These results indicated that the various components of the complete reaction could act together to conserve the integrity of the full-length gene through DNA mismatch resolution.

Another matrix experiment was conducted to expand on these results and to identify additional conditions for DNA mismatch resolution for this synthetic system. 60 nanograms of cycle3/wild-type GFP heteroduplex were treated with CEL I and T4 DNA polymerase at various concentrations in the presence of 2.5 units of Taq DNA polymerase and 0.2 units of T4 DNA ligase in 1×NEB T4 DNA ligase buffer containing 0.5 mM of each dNTP in a reaction volume of 10 microliters. Each set of reactions was incubated for 1 hour at 20° C., 30° C., 37° C., or 45° C. All reactions were then run on a 1.5% TBE-agarose gels in the presence of ethidium bromide. The results showed that the GFP heteroduplex was cleaved into discrete fragments by the CEL I preparation alone. The success of DNA mismatch resolution was initially gauged by the degree to which the apparent full-length integrity of the GFP sequence was maintained by the other components of the mismatch resolution system in the presence of CEL I. Conditions of enzyme concentration and temperature were identified that conserved a high proportion of the DNA as full-length molecules in this assay. Namely, one microliter of the CEL I fraction five preparation (described in Example 1) with one microliter (1 unit) of the T4 DNA polymerase in the presence of the other reaction components which were held constant in the experiment. It was found that as the reaction temperature increased, the degradative activity of CEL I increased accordingly. Furthermore, it was shown that the other components of the repair reaction acted to conserve the integrity of the full-length DNA at 20° C., 30° C., and 37° C., but was remarkably less efficient at conserving the full-length DNA at 45° C. From these results, we concluded that under these experimental conditions, incubation at 45° C. was not optimal for the process of GRAMMR, and that incubation at 20° C., 30° C., and 37° C. were permissible.

Another experiment was performed in which alternative enzymes were used for the DNA mismatch resolution reaction. Instead of T4 DNA ligase, Taq DNA ligase was used. Pfu DNA polymerase (Stratagene) was employed in a parallel comparison to a set of reactions that contained T4 DNA polymerase as the 3′ exonuclease/polymerase. Reactions were carried out in Taq DNA ligase buffer containing 8 units of Taq DNA ligase (NEB), 2.5 units Taq DNA polymerase, 0.5 mM of each dNTP, various dilutions of CEL I, and either T4 DNA polymerase or Pfu DNA polymerase). Reactions were run on a 1.5% TBE-agarose gels in the presence of ethidium bromide. It was found that in the presence of the Pfu DNA polymerase, Taq DNA polymerase, and Taq DNA ligase, the full-length integrity of the CEL I-treated substrate DNA was enhanced compared to DNA incubated with CEL I alone. This result shows that enzymes with functionally equivalent activities can be successfully substituted into the GRAMMR reaction.

### EXAMPLE 3

### Restoration of Restriction Sitesto GFP Heteroduplex DNA after DNA Mismatch Resolution (GRAMMR)

This experiment teaches the operability of genetic reassortment by DNA mismatch resolution (GRAMMR) by demonstrating the restoration of restriction sites.

The full-length products of a twenty-fold scale-up of the GRAMMR reaction, performed at 37° C. for one hour, using the optimal conditions found above (the 1× reaction contained sixty nanograms of heteroduplex DNA, one microliter of CEL I fraction five (described in Example 1), one unit T4 DNA polymerase in the presence of 2.5 units of Taq DNA polymerase and 0.2 units of T4 DNA ligase in 1×NEB T4 DNA ligase buffer containing 0.5 mM of each dNTP in a reaction volume of 10 microliters) were gel-isolated and subjected to restriction analysis by endonucleases whose recognition sites overlap with mismatches in the GFP heteroduplex, thereby rendering those sites in the DNA resistant to restriction enzyme cleavage. The enzymes used were BamHI, HindIII, HpaI, and XhoI. Negative controls consisted of untreated GFP heteroduplex. Positive controls consisted of Cycle 3 or wild type GFP sequences, individually. All controls were digested with the same enzymes as the product of the DNA mismatch resolution reaction. All samples were run on a 2% TBE-agarose gel in the presence of ethidium bromide.

After treatment with the mismatch resolution cocktail, a proportion of the DNA gained sensitivity to BamHI and XhoI restriction endonucleases, indicating that DNA mismatch resolution had occurred. The HpaI-cut samples could not be interpreted since a low level of cleavage occurred in the negative control. The HindIII, BamHI and XhoI sites displayed different degrees of cleavage in the GRAMMR-treated samples. Restoration of the XhoI site was more extensive than that of the BamHI site, which was in turn, more extensive than restoration at HindIII site.

The extent to which cleavage occurs is indicative of the extent to which mismatches in the DNA have been resolved at that site. Differences in mismatch resolution efficiency may relate to the nature or density of mismatches present at those sites. For example, the XhoI site spans a three-mismatch cluster, whereas the BamHI site spans two mismatches and the HindIII site spans a single mismatch.

### EXAMPLE 4

### GRAMMR-Treated GFP Genes

This example demonstrates that GRAMMR can reassort sequence variation between two gene sequences in a heteroduplex and that there are no significant differences in GRAMMR products that were directly cloned, or PCR amplified prior to cloning.

The GRAMMR-treated DNA molecules of Example 3 were subsequently either directly cloned by ligation into pCR-Blunt II-TOPO (Invitrogen), or amplified by PCR and ligated into pCR-Blunt II-TOPO according to the manufacturer's instructions, followed by transformation into E. coli. After picking individual colonies and growing in liquid culture, DNA was prepared and the sequences of the GFP inserts were determined. As negative controls, the untreated GFP heteroduplex substrate was either directly cloned or PCR amplified prior to cloning into the plasmid.

In GRAMMR, reassortment of sequence information results from a process of information transfer from one strand to the other. These sites of information transfer are analogous to crossover events that occur in recombination-based DNA shuffling methods. For the purposes of relating the results of these reassortment experiments, however, the GRAMMR output sequences are described in terms of crossovers. Sequences of twenty full-length GFP clones that were derived from the GRAMMR-treated GFP genes were analyzed. Four of these clones were derived from DNA that had been directly cloned into pZeroBlunt (Invitrogen) following GRAMMR reaction (no PCR amplification). The other sixteen sequences were cloned after PCR amplification. Analysis of these full-length GFP sequences revealed that all twenty sequences had undergone sequence reassortment having between one and ten crossovers per gene. A total of 99 crossovers were found in this set of genes, giving an average of about 5 crossovers per gene. With the distance between the first and last mismatches of about 590 nucleotides, an overall frequency of roughly one crossover per 120 base-pairs was calculated. Within this set of twenty clones, a total of seven point mutations had occurred within the sequences situated between the PCR primer sequences, yielding a mutation frequency of roughly 0.05%.

Thirty-five clones that had not been subjected to the GRAMMR reaction were sequenced. Of these controls, fourteen were derived from direct cloning and twenty-one were obtained after PCR amplification using the GFP heteroduplex as template. Of these thirty-five non-GRAMMR treated control clones, eight were recombinants, ranging from one to three crossovers, with most being single crossover events. A total of twenty-five point mutations had occurred within the sequences situated between the PCR primers, yielding a mutation frequency of roughly 0.1%.

No significant differences were observed between the GRAMMR-treated products that were either directly cloned or PCR amplified. Notably, though, in the non-GRAMMR-treated controls, the frequency of recombinants was higher in the PCR amplified DNAs than in the directly cloned DNAs. This higher frequency is consistent with results obtained by others in which a certain level of recombination was found to be caused by “jumping PCR.” (Paabo, et al., DNA damage promotes jumping between templates during enzymatic amplification. J Biol Chem 265(90)4718-4721).

### EXAMPLE 5

### Heteroduplex Substrate Preparation for Plasmid-on-Plasmid Genetic Reassortment By DNA Mismatch Resolution (POP GRAMMR) of GFP Plasmids

This example teaches that heteroduplex substrate for Genetic Reassortment by DNA Mismatch Resolution can be in the form of intact circular plasmids. Cycle 3-GFP and wild-type GFP heteroduplex molecules were prepared plasmid-on-plasmid (POP) format. In this format, the GFP sequences were reasserted within the context of a circular double-stranded plasmid vector backbone. This made possible the recovery of the reasserted product by direct transformation of E. coli using an aliquot of the GRAMMR reaction. Consequently, neither PCR amplification nor other additional manipulation of the GRAMMR-treated DNA was necessary to obtain reasserted clones.

Mismatched DNA substrate for POP-GRAMMR reactions was generated containing wild-type GFP (SEQ ID NO: 29) and Cycle 3 GFP (SEQ ID NO: 30), resulting in the two pBluescript-based plasmids, pBSWTGFP (SEQ ID NO: 31) and pBSC3GFP (SEQ ID NO: 17), respectively. The GFPs were inserted between the KpnI and EcoRI sites of the pBluescript polylinker so that the only sequence differences between the two plasmids occurred at sites where the wild-type and Cycle 3 GFPs differ from one-another. Both plasmids were linearized by digestion of the plasmid backbone with SapI, cleaned up using a DNA spin-column, mixed, amended to 1×PCR buffer (Barnes, 1994; PNAS, 91, 2216-2220), heated in a boiling water bath for three minutes, and slow-cooled to room temperature to anneal the denatured DNA strands. Denaturing and annealing these DNAs led to a mixture of duplexes; the re-formation of parental duplexes, and the formation of heteroduplexes from the annealing of strands from each of the two input plasmids. Parental duplexes were deemed undesirable for GRAMMR and were removed by digestion with restriction enzymes that cut in one or the other parental duplex but not in the heteroduplexed molecules. PmlI and XhoI were chosen for this operation since PmlI cuts only in the wild-type GFP sequence and XhoI cuts only Cycle 3 GFP. After treatment with these enzymes, the products were resolved on an agarose gel. The full-length, uncut heteroduplex molecules were resolved from the PmlI- and XhoI-cut parental homoduplexes in an agarose gel and purified by excision of the band and purification with a DNA spin column.

The resulting population of heteroduplexed molecules was treated with DNA ligase to convert the linear DNA into circular, double-stranded DNA heteroduplexes. After confirmation by agarose gel-shift analysis, the circular double-stranded GFP heteroduplexed plasmid was used as substrate for GRAMMR reactions. Examples of the resulting clones are included as SEQ ID NO: 5, SEQ ID NO: 6, SEQ ID NO: 7, and SEQ ID NO: 8.

### EXAMPLE 6

### Exemplary Reaction Parameters for Genetic Reassortment by DNA Mismatch Resoluton: CEL I and T4 DNA Polymerase Concentrations Compared

The GRAMMR reaction involves the interaction of numerous enzymatic activities. Several parameters associated with the GRAMMR reaction were examined, such as CEL I concentration, T4 DNA polymerase concentration, reaction temperature, substitution of T4 DNA polymerase with T7 DNA polymerase, the presence of Taq DNA polymerase, and the source of the CEL I enzyme. A matrix of three different CEL I concentrations versus two concentrations of T4 DNA polymerase was set up to examine the limits of the in vitro DNA mismatch resolution reaction.

Twenty-one nanograms (21 ng) of the circular double-stranded heteroduplexed plasmid, prepared as described in example 5, was used as substrate in a series of ten microliter reactions containing 1×NEB ligase buffer, 0.5 mM each dNTP, 1.0 unit Taq DNA polymerase, 0.2 units T4 DNA ligase (Gibco/BRL), either 1.0 or 0.2 units T4 DNA polymerase, and either 0.3, 0.1, or 0.03 microliters of a CEL I preparation (fraction 5, described in Example 1). Six reactions representing all six combinations of the two T4 DNA polymerase concentrations with the three CEL I concentrations were prepared, split into equivalent sets of five microliters, and incubated at either 20 degrees C. or 37 degrees C. A control reaction containing no CEL I and 0.2 unit of T4 DNA polymerase with the other reaction components was prepared and incubated at 37 degrees C. After 30 minutes, one microliter aliquots of each reaction were transformed into competent DH5-alpha E. coli which were then plated on LB amp plates. Colonies were picked and cultured. Plasmid DNA was extracted and examined by restriction fragment length polymorphism analysis (RFLP) followed by sequence analysis of the GFP gene sequences. RFLP analysis was based on differences in several restriction enzyme recognition sites between the wild-type and Cycle 3 GFP genes. The RFLP results showed that throughout the CEL I/T4 DNA polymerase/temperature matrix, reassortment of restriction sites, that is GRAMMR, had occurred, and that no such reassortment had occurred in the zero CEL I control clones. DNA sequence analysis confirmed that reassortment had occurred in all of the CEL I -containing samples. Sequencing also confirmed that the zero- CEL I controls were not reassorted, with the exception of a single clone of the 16 control clones, which had a single-base change from one gene sequence to the other, presumably resulting either from repair in E. coli or from random mutation. The sequences of several exemplary GRAMMR-output GFP clones are shown; all of which came from the reaction containing 0.3 microliters of the CEL I preparation and 1.0 unit of T4 DNA polymerase incubated at 37 degrees C. The parental wild-type and Cycle 3 GFP genes are shown first for reference.

### EXAMPLE 7

### Taq DNA Polymerase is Not Required for Genetic Reassortment by DNA Mismatch Resolution

This experiment teaches that Taq DNA Polymerase does not dramatically, if at all, contribute or interfere with the functioning of GRAMMR. Taq DNA polymerase is reported to have a 5′ flap-ase activity, and had been included in the teachings of the previous examples as a safeguard against the possible formation and persistence of undesirable 5′ flaps in the heteroduplexed DNA undergoing the GRAMMR reaction.

GRAMMR reactions were set up, as in Example 6, with twenty-one nanograms of the circular double-stranded heteroduplexed GFP plasmid substrate in ten microliter reactions containing 1×NEB ligase buffer, 0.5 mM each dNTP, 0.2 units T4 DNA ligase, 1.0 unit T4 DNA polymerase, 1.0 microliter of a CEL I preparation (fraction 5, described in Example 1), and either 2.5 units, 0.5 units of Taq DNA polymerase, or no Taq DNA polymerase. After 30 minutes, one microliter aliquots of each reaction were transformed into competent DH5-alpha E. coli which were then plated on LB amp plates. Colonies were picked and cultured. Plasmid DNA was extracted and examined by RFLP analysis followed by sequence analysis of the GFP gene sequences. The RFLP results showed that reassortment of restriction sites, that is, GRAMMR, had occurred both in the presence and the absence of Taq DNA polymerase in the GRAMMR reaction. DNA sequence analysis confirmed these results. Therefore, the data shows that Taq DNA polymerase was unnecessary for GRAMMR.

### EXAMPLE 8

### Alternate Proofreading DNA Polymerases for Genetic Reassortment by DNA Mismatch Resolution

This experiment teaches that Genetic Reassortment by DNA Mismatch Resolution is not limited to the use of T4 DNA polymerase, and that alternate DNA polymerases can be substituted for it.

Reactions were set up, as in Example 6, with twenty-one nanograms of the circular double-stranded heteroduplexed GFP plasmid substrate in ten microliter reactions containing 1×NEB ligase buffer, 0.5 mM each DNTP, 0.2 units T4 DNA ligase (Gibco/BRL), 10 units or 2 units of T7 DNA polymerase, 1.0 microliter of a CEL I preparation (fraction 5, described in Example 1), and 2.5 units of Taq DNA polymerase. After 30 minutes, one microliter aliquots of each reaction were transformed into competent DH5-alpha E. coli which were then plated on LB amp plates. Colonies were picked and cultured. Plasmid DNA was extracted and examined by RFLP analysis followed by sequence analysis of the GFP gene sequences. The RFLP results showed that reassortment of restriction sites, that is GRAMMR, had occurred in both T7 DNA polymerase-containing reactions. DNA sequence analysis confirmed these results. Therefore, the data shows that T7 DNA polymerase can substitute for T4 DNA polymerase for GRAMMR. In addition, it shows that individual components and functionalities can be broadly substituted in GRAMMR, while still obtaining similar results.

### EXAMPLE 9

### Use of Cloned CEL I in the GRAMMR Reaction

This example teaches that CEL I from a cloned source can be used in place of native CEL I enzyme purified from celery in Genetic Reassortment By DNA Mismatch Resolution without any noticeable change in results.

The cDNA of CEL I was cloned from celery RNA. The gene was inserted into a TMV viral vector and expressed. Transcripts of the construct were used to infect Nicotiana benthamiana plants. Infected tissue was harvested, and the CEL I enzyme was purified. The results of the GRAMMR reaction obtained using the purified enzyme were compared to those using CEL I purified from celery, and were found to be similar.

Reactions were set up using twenty-one nanograms of the circular double-stranded heteroduplexed GFP plasmid substrate, as described in Example 5, in ten microliters containing 1×NEB ligase buffer, 0.5 mM each dNTP, 0.2 units T4 DNA ligase (Gibco/BRL), 1 unit of T4 DNA polymerase, and either 1.0 microliter of CEL I purified from celery (fraction 5, described in Example 1), or 0.3 microliters of CEL I purified from a cloned source. After 30 minutes, one microliter aliquots of each reaction were transformed into competent DH5-alpha E. coli which were then plated on LB amp plates. Colonies were picked and cultured. Plasmid DNA was extracted and examined by RFLP analysis followed by sequence analysis of the GFP gene sequences. The RFLP results showed that reassortment of restriction sites, that is, GRAMMR had occurred in both celery-derived CEL I, as well as cloned CEL I-containing reactions. DNA sequence analysis confirmed these results. Therefore, the data shows CEL I from a cloned source can be used in lieu of CEL I from celery for GRAMMR. In addition, the data demonstrates that it is CEL I activity that is part of the GRAMMR reaction, rather than a coincidental effect resulting from the purifying steps used in extracting CEL I from celery.

### EXAMPLE 10

### Molecular Breeding of Tobamovirus 30K Genes in a Viral Vector

In the preceding examples, Genetic Reassortment by DNA Mismatch Resolution has been taught to be useful for reassorting sequences that are highly homologous, for example, wtGFP and Cycle 3 GFP are 96% identical. The present example teaches that GRAMMR can be used to reassort more divergent nucleic acid sequences, such as genes encoding tobamovirus movement protein genes.

Heteroduplexes of two tobamovirus movement protein (MP) genes that are approximately 75% identical were generated. The heteroduplex substrate was prepared by annealing partially-complementary single-stranded DNAs of opposite strandedness synthesized by asymmetric PCR; one strand encoding the movement protein gene from the tobacco mosaic virus U1 type strain (TMV-U1) (SEQ ID NO: 9), and the other strand encoding the movement protein gene from tomato mosaic virus (ToMV) (SEQ ID NO: 10). The sequences of the two partially complementary movement protein genes were flanked by 33 nucleotides of absolute complementarity to promote annealing of the DNAs at their termini and to facilitate PCR amplification and cloning. The annealing reaction took place by mixing 2.5 micrograms of each single-stranded DNA in a 150 microliter reaction containing 333 mM NaCl, 33 mM MgCl2, 3.3 mM dithiothreitol, 166 mM Tris-HCl, pH 7, and incubating at 95° C. for one minute followed by slow cooling to room-temperature. GRAMMR was performed by incubating 5 microliters of the heteroduplex substrate in a 20 microliter reaction containing 1×NEB ligase buffer, 0.5 mM each dNTP, 0.4 units T4 DNA ligase (Gibco/BRL), 2.0 units of T4 DNA polymerase, and CEL I. The CEL I was from a cloned preparation and the amount that was used varied from 2 microliters of the prep, followed by five serial 3-fold dilutions. A seventh preparation with no CEL I was prepared, which served as a control.

After one hour at room-temperature, DNA was purified from the reactions using Strataprep spin DNA purification columns (Stratagene, LaJolla, Calif.) and used as templates for PCR reactions using primers designed to anneal to the flanking primer-binding sites of the two sequences. PCR products from each reaction were purified using Strataprep columns, digested with AvrII and PacI, and ligated into the movement protein slot of similarly-cut pGENEWARE®-MP-Avr-Pac. This plasmid contained a full-length infectious tobamovirus-GFP clone modified with AvrII and PacI sites flanking the movement protein gene to permit its replacement by other movement protein genes. After transformation of DH5-alpha E. coli and plating, colonies were picked, cultures grown, and DNA was extracted. The movement protein inserts were subjected to DNA sequence analysis from both directions and the sequence data confirmed that in the majority of inserts derived from the GRAMMR-treated material were reassorted sequences made up of both TMV-U1 and ToMV movement protein gene sequences. The DNA sequences of several exemplary GRAMMR output MP clones are shown as SEQ ID NO: 11, SEQ ID NO: 12, SEQ ID NO: 13, SEQ ID NO: 14, and SEQ ID NO: 15.

### EXAMPLE 11

### GRAMMR to Generate Improved Arsenate Detoxifying Bacteria

Arsenic detoxification is important for mining of arsenopyrite-containing gold ores and other uses, such as environmental remediation. Plasmid pGJ103, containing an arsenate detoxification operon (Ji and Silver, 1992)(Ji, G. and Silver, S., Regulation and expression of the arsenic resistance operon from Staphylococcus aureus plasmid pI258, J. Bacteriol. 174, 3684-3694 (1992), incorporated herein by reference), is obtainable from Prof. Simon Silver (U. of Illinois, Chicago, Ill.). E. coli TG1 containing pGJ103, containing the pI258 ars operon cloned into pUC19, has a MIC (minimum inhibitory concentration) of 4 μg/ml on LB ampicillin agar plates. The ars operon is amplified by mutagenic PCR, cloned into pUC19, and transformed into E. coli TG1. Transformed cells are plated on a range of sodium arsenate concentrations (2, 4, 8, 16 mM). Colonies from the plates with the highest arsenate levels are picked. The colonies are grown in a mixed culture with appropriate arsenate selection. Plasmid DNA is isolated from the culture. The plasmid DNA is linearized by digestion with a restriction endonuclease that cuts once into the pUC19 plasmid backbone. The linearized plasmids are denatured by heating 10 min. at 94° C. The reaction is allowed to cool to promote annealing of the single strands. Partially complementary strands that hybridize have non-base paired nucleotides at the sites of the mismatches. Treatment with CEL I (purified by the method of Example 9) causes nicking of one or the other polynucleotide strand 3′ of each mismatch. The presence of a polymerase containing a proofreading activity, such as T4 DNA polymerase allows excision of the mismatch, and subsequent 5′-to-3′ polymerase activity fills in the gap using the other strand as a template. T4 DNA ligase then seals the nick by restoring the phosphate backbone of the repaired strand. The result is a randomization of mutations among input strands to give output strands with potentially improved properties. These output polynucleotides are transformed directly into E. coli TG1 and the cells are plated at higher arsenate levels; 8, 16, 32, 64 mM. Colonies are picked from the plates with the highest arsenate levels and another round of reassortment is performed as above except that resulting transformed cells are plated at 32, 64, 128, 256 mM arsenate. The process can then be repeated one or more times with the selected clones in an attempt to obtain additional improvements.

### EXAMPLE 12

### Cloning, Expression and Purification of CEL I Endonuclease

This example teaches the preparation of nucleic acid molecules that were used for expressing CEL I endonuclease from plants, identified herein as, p1177MP4-CEL I Avr (SEQ ID NO: 1), and p1177MP4-CEL I 6HIS (SEQ ID NO: 2). In particular, this example refers to disclosures taught in U.S. Pat. Nos. 5,316,931, 5,589,367, 5,866,785, and 5,889,190, incorporated herein by reference.

Celery RNA Extraction:

Celery was purchased from a local market. Small amounts of celery tissue (0.5 to 0.75 grams) were chopped, frozen in liquid nitrogen, and ground in a mortar and pestle in the presence of crushed glass. After addition of 400 microliters of Trizol and further grinding, 700 microliters of the extract were removed and kept on ice for five minutes. Two hundred microliters of chloroform were then added and the samples were centrifuged, left at room temperature for three minutes, and re-centrifuged at 15,000 g for 10 minutes. The aqueous layer was removed to a new tube and an equal volume of isopropanol was added. Tubes were inverted to mix and left at room temperature for 10 minutes followed by centrifugation at 15,000 g for ten minutes at 4° C. The pellet was washed twice in 400 microliters of 70% ethanol, once in 100% ethanol, air dried, and resuspended in 40 microliters of distilled water. One microliter of RNasin was added and 3.5 microliters was run on a 1% agarose gel to check the quality of the RNA prep (Gel picture). The remainder was stored at −70° C. until further use.

CEL I Gene Cloning and Expression by a Viral Vector:

The total RNA from celery was subjected to reverse transcription followed by PCR to amplify the cDNA encoding the CEL I gene sequence. In separate reactions, eleven microliters of the total celery RNA prep was mixed with one microliter (50 picomoles) of either CelI-Avr-R, CelI-6H-R, or with two microliters of oligo dT primer. CelI-Avr-R was used to prime cDNA and amplify the native CEL I sequence at the 3′ end of the gene, while CelI-6H-R was used to add a sequence encoding linker peptide and a 6-His tag to the 3′ terminus of the CEL I gene. The samples were heated to 70° C. for one minute and quick-chilled on ice prior to the addition of 4 microliters of 5× Superscript II buffer, two microliters of 0.1 M DTT, 1 microliter of 10 mM each dNTP, and 1 microliter of Superscript II (Gibco/BRL) to each reaction. The reactions were incubated at 42° C. for one hour.

PCR amplification of the CEL I cDNA sequence was performed using the method of W. M. Barnes (Proc Natl Acad Sci. USA, 1994 Mar 15;91(6):2216-20) with a Taq-Pfu mixture or with Pfu alone. The RT reaction primed with CelI-Avr-R was used as template for a PCR using primers CelI-Pac-F (as the forward primer) paired with CelI-Avr-R (as the reverse primer). In other PCRs, the RT reaction that was primed with oligo dT was used as template for both of the above primer pairs. All PCR reactions were performed in 100 microliters with 30 cycles of annealing at 50° C. and two minutes of extension at 72° C. Aliquots of the resulting reactions were analyzed by agarose gel electrophoresis. Reactions in which Pfu was used as the sole polymerase showed no product. All reactions performed with the Taq/Pfu mixtures yielded product of the expected size. However, those amplified from cDNA primed with Cel I specific primer pairs gave more product than reactions amplified from cDNA primed with oligo-dT. DNAs from the PCR reactions that gave the most product were purified using a Zymoclean DNA spin column kit and digested with PacI and AvrII, gel-isolated, and ligated into PacI and AvrII-digested plasmid pRT130, a tobamovirus-based GENEWARE® vector. 2 microliters of each ligation were transformed into DH5α competent E. coli and cultured overnight on LB-amp agar plates. Colonies were picked and grown overnight in liquid culture, and plasmid DNA was isolated using a Qiagen plasmid prep kit. 12 clones from each construct were screened by digestion with PacI and AvrII and 11 of 12 of each set were positive for insert of the correct size. Ten of the clones for each construct were transcribed in-vitro and RNA was inoculated to N. benthamiana plants. In addition, the CEL I gene inserts in both sets of ten clones were subjected to sequence analysis. Several clones containing inserts encoding the native form of CEL I had sequence identical to the published CEL I sequence in WO 01/62974 A1. One clone containing an insert encoding CEL I fused to a 6-Histidine sequence was identical to the published CEL I sequence. One clone of each (pRT130-CEL I Avr-B3 and pRT130-CEL I 6His-A9, respectively) was selected for further work. The CEL I-encoding sequences in these clones were subsequently transferred to another GENEWARE® vector. The sequences of these clones, p1177MP4-CEL I Avr-B3, and p1177MP4-CEL I 6His-A9 are provided as SEQ ID NO: 1 and SEQ ID NO: 2, respectively.

Assay of Cloned CEL I Activities:

To determine whether the GENEWARE® constructs containing Cel I sequences could produce active CEL I enzyme, samples of pRT130-CEL I Avr (SEQ ID NO: 1) and pRT130-CEL I 6His (SEQ ID NO: 2), and GFP-GENEWARE control-infected plants were harvested and homogenized in a small mortar and pestle in Tris-HCl at pH 8.0. Extracts were clarified and assayed for supercoiled DNA nicking activity. Each supercoiled DNA nicking assay was performed in a reaction containing 0.5 micrograms of a supercoiled plasmid prep of a pUC19-derivative in 1×NEB ligase buffer in a total volume of 10 microliters. The amounts of plant extract added to the reactions were 0.1 microliter, 0.01 microliter, or 0.001 microliter, incubated at 42° C. for 30 minutes, and run on a 1% TBE-agarose gel in the presence of ethidium bromide. Little or no nicking activity was detected in the GFP-GENEWARE control-infected plant extract whereas extracts from plants infected with the CEL I-GENEWARE constructs showed appreciable amounts of activity against the plasmid DNA substrate.

Additional activity assays were performed on extracts of plants inoculated with pRT130-CEL I Avr-B3 and pRT130-CEL I 6His-A9. In these assays, intracellular fluid was washed from infected leaves and assayed separately from material obtained from the remaining washed leaf tissues. Assays were performed as described above with the exception that the incubation was at 37° C. for one hour. Samples were run on a 1% TBE-agarose gel in the presence of ethidium bromide and photographed.

Purification of 6His-tagged CEL I from Infected N. benthamiana Plants:

N. benthamiana plants were inoculated with RNA transcripts from pRT130-CEL I 6His-A9 at 20-21 days post-sowing. Tissues were harvested from 96 infected plants at 10 days post-inoculation and subjected to intracellular fluid washes. Briefly, infected leaf and stem material was vacuum infiltrated for 30 seconds twice with chilled infiltration buffer (50 mM phosphate pH 4 in the presence of 7 mM β-ME). Infiltrated tissues were blotted to adsorb excess buffer and secreted proteins were recovered by centrifugation at 2500×g for 20 min using basket rotor (Beckman). PMSF was added to the extracted intracellular fluid (IF) containing recombinant CEL_I to a final concentration of 1 mM, and incubated at 25° C. for 15 min with stirring. After addition of Imidazole (pH 6.0) and NaCl to the extract to the final concentration of 5 mM and 0.5 M respectively, IF was adjusted to pH 5.2 and filtered through 1.2μ Sartorius GF membrane (Whatman) to remove most of the Rubisco and green pigments. Immediately after clarification, pH was adjusted to 7.0 using concentrated NaOH solution and incubated on ice for 20 min to allow non-proteinaceous material to precipitate. IF was further clarified using 0.8μ or 0.65/0.45μ Sartorius G F (Whatman). Recombinant CEL I was purified from the clarified IF by metal chelating affinity chromatography using Ni²⁺ Fast Flow Sepharose (Amersham Pharmacia Biotech, NJ) equilibrated with binding buffer (50 mM phosphate, 0.5 M NaCl; pH 7.0) containing 5 mM imidazole, with a linear velocity of 300 cm/hr. Unbound protein was washed with 20 mM imidazole/binding buffer, and CEL I was eluted from Ni²⁺ Sepharose with a linear gradient of 20 to 400 M imidazole in the binding buffer. Fractions still containing imidazole were assayed for supercoiled DNA nicking activity as described above but were found to have negligible activity. The same fractions were then dialyzed against 0.1 M Tris-HCl, pH 8.0 in the presence of ZnCl₂ using 10 kD MWCOF dialysis tubing (Pierce) and assayed again. The supercoiled DNA nicking activity was restored after this dialysis.

IF and purified CEL-I protein were analyzed using Sodium Dodecyl Sulfate Polyacrylamide Gel Electrophoresis (SDS-PAGE) precast Tris-glycine gels (Invitrogen, Carlbad, Calif.) in the buffer system of Laemmli with a Xcell II Mini-Cell apparatus (Invitrogen, Carlsbad, Calif.). The protein bands were visualized by Coomassie brilliant blue and by silver staining. SDS-PAGE Gels were scanned and analyzed using Bio-Rad gel imager.

Mass Spectrometry of Purified CEL I:

The average molecular mass of the purified CEL I was determined by matrix-assisted laser/desorption ionization time-of-flight mass spectrometry (MALDI-TOF). An aliquot of CEL I was diluted 1:10 with 50% acetonitrile/water and mixed with sinapinic acid matrix (1:1 v/v) using a PE Biosystem DE-Pro mass spectrometer. The mass spectrometry was performed using an accelerating voltage of 25 kV and in the positive-linear ion mode.

Mass Spectrometry of Peptides Isolated from Purified CEL I:

CEL I was separated on SDS-PAGE on a 14% gel and stained with Coomassie brilliant blue. A single homogenous band was visible. This band was excised and de-stained completely. Protein was reduced in the presence of 10 mM DDT in 50% acetonitrile for 30 min at 37° C. and reduced sulfhydro groups were blocked in the presence of 28 mM iodoacetamide in 50% acetonitrile for 30 min at 24° C. in absence of light. Gel pieces were washed with 50% acetonitrile and after partial dehydration, the excised CEL I band was macerated in a solution of high purity trypsin (Promega). The proteolytic digestion was allowed to continue at 37° C. for 16 h. The resulting peptides were eluted from gel pieces with a 50% acetonitrile and 0.1 % tri-fluoro-acetic acid (TFA) concentrated in a SpeedVac. The peptides were analyzed by MALDI-TOF. Mixed tryptic digests were crystallized in a matrix of α-cyano-4-hydroxycinnamic acid and analyzed by using a PerSeptive Biosystem DE-STR MALDI-TOF mass spectrometer equipped with delayed extraction operated in the reflector-positive ion mode and accelerating voltage of 20 kV. Expected theoretical masses were calculated by MS-digest (Protein Prospector) or GPMAW program (Lighthouse Data, Odense, Denmark). For tandem mass spectrometry (nano electrospray ionization (ESI), peptide samples were diluted with 5% acetonitrile/0.1% formic acid and subjected to LC MS/MS, analyzed on a quadropole orthogonal time-of-flight mass spectrometry instrument (micromass, inc., Manchester, UK). The data were processed by Mslynx and database was searched by Sonar.

Virally expressed, recombinant CEL I was secreted to the IF. Clarified IF-extracted material was used to purify the His-tag CEL I activity. CEL I was purified using one step Ni²⁺ affinity chromatography separation. A highly purified homogeneous single protein band was purified as determined by Coomassie stained SDS-PAGE and mass spectrometry. The size of mature proteins and percent glycosylation concur with what has been reported for the CEL I protein isolated from celery (Yang et al., 2000). The purified CEL I has an average molecular mass of 40 kD as determined by MALDI-TOF mass spectrometry, indicates 23.5% glycosylation by mass. CEL I has four potential glycosylation cites at amino acid positions 58, 116, 134, and 208. A mono-isotopic mass of 2152.6086 (2152.0068 Theoretical) Da corresponding to the mass of the peptide 107-125 (K)DMCVAGAIQNFTSQLGHFR(H) (SEQ ID NO: 35) that was recovered by MALDI-TOF, indicates that asparagine 116 is not glycosylated. Together, these gel analyses and mass spectrometry data indicate that a significant fraction of the CEL I protein was recoverable, and that the protein was correctly processed in the N. benthamiana plant. For subsequent experiments, the 6-His tagged CEL I gene was produced using p1177MP4-CEL I 6His-A9. This clone was transcribed and inoculated onto N. benthamiana plants, which were harvested 8 days post infection. The plant material was combined with 2 volumes of extraction buffer (500 mM NaCl, 100 mM NaPi, 25 mM Tris pH 8.0, 7 mM Beta-mercaptoethanol, 2 mM PMSF) and vacuum infiltrated. Following buffer infiltration the tissue was macerated in a juice extractor, the resulting green juice adjusted to 4% w/v polyethyleneglycol, and let stand at 4° C. for one hour. The green juice was clarified by either centrifugation at low speed (3500×g) for 20 minutes or combined with perlite (2% w/v) and filtered through a 1.2 μm filter. The tagged CEL I can be selectively purified from the clarified green juice by metal affinity chromatography. The green juice was either combined with nickel-NTA resin, and batch binding of the CEL I performed, or purification was performed in column format, where the green juice was permitted to flow through a bed of nickel-NTA resin. For binding, the clarified green juice was adjusted to 10% w/v glycerol and 10 mM imidazole. Following binding the resin was washed extensively with wash buffer (330 mM NaCl, 100 mM NaPi, pH 8.0, 10 mM imidazole) and the bound CEL I enzyme eluted from the nickel-NTA resin in 2 resin-bed volumes of 1× phosphate-buffered saline (PBS) containing 400 mM imidazole. The CEL I preparation was subsequently dialyzed against 1×PBS to remove the imidazole, assayed for activity, and stored at 4 ° C. or at −20 ° C. with or without glycerol until use.

### EXAMPLE 13

### Cloning, Expression and Use of Res I Endonuclease

This example teaches the construction of a cDNA library from Selaginella lepidophylla, the identification of a nucleic acid sequence from the library that encodes an endonuclease, and the expression of the new endonuclease, herein designated as “RES I.”

RNA was extracted from tissues of the resurrection plant, Selaginella lepidophylla, using the Trizol method, and oligo-dT primed cDNA that was prepared using standand methodology. Resulting cDNAs were ligated into a GENEWARE®-based cloning vector and the ligation products were transformed into competent E. coli cells. Bacterial colonies containing GENEWARE® cDNA clones were picked at random and grown as liquid cultures prior to DNA prepping and determination of the cloned cDNA sequences. The sequence files for the cloned Selaginella cDNAs were loaded into a database which was then searched by BLAST analysis for sequences that had similarity to the DNA sequence of the CEL I gene. BLAST analysis was also performed on other DNA sequence databases containing sequences of cDNAs obtained from other species.

BLAST hits that showed some level of homology to the celery CEL I sequence were identified in libraries from several species and the corresponding GENEWARE®-cDNA clones were re-arrayed into a single set of GENEWARE®-cDNA clones. This set of cDNA clones was then transcribed in vitro to generate infectious GENEWARE® transcripts which were then inoculated onto leaves on Nicotiana benthamiana plants for expression analysis of the cDNA sequences encoded within the GENEWARE® viral genome. At seven days post-inoculation, leaf samples were taken from the infected plants and homogenized in two volumes of water. The extracts were then assayed for supercoiled DNA nicking and cleavage activity.

Each supercoiled DNA nicking assay was performed in a reaction containing 0.5 micrograms of a supercoiled plasmid prep of a pUC19-derivative in 1×NEB T4 DNA ligase buffer in a total volume of 10 microliters. The amounts of plant extract added to the reactions were 1 microliter, 0.33 microliter, or 0.011 microliter, incubated at 37° C. for 30 minutes, and run on a 1% TAE-agarose gel in the presence of Gelstar fluorescent DNA staining reagent. Little or no nicking activity was detected in uninfected plant extracts whereas only extracts from plants infected with GENEWARE® constructs containing cDNAs for a single gene from Selaginella lepidophylla showed appreciable amounts of activity against the plasmid DNA substrate.

The complete gene sequences of these clones were determined and PCR primers were designed to amplify the open reading frame minus any non-coding 5′ and 3′ sequences and to add a six histidine tail to the C-terminus of the encoded protein. The primers were then used to amplify the ORF from one of the active full-length Selaginella clones. The resulting PCR product was then cloned into the GENEWARE® vector pDN4 between the PacI and AvrII sites for expression in planta. The resulting clone, pLSB2225, which contains the RES I ORF (SEQ ID NO: 16), and which encodes the RES I protein (SEQ ID NO: 34), was sequenced to confirm that the gene had been inserted correctly, and then transcribed in vitro followed by inoculation of the infectious transcripts onto N. benthamiana plants. Seven days post inoculation, infected plant extracts were made as above and assayed for supercoiled DNA nicking and digestion activity to confirm the activity of the cloned enzyme.

Each supercoiled DNA nicking assay was performed in a reaction containing 0.5 micrograms of a supercoiled plasmid prep of a pUC19-derivative in 1×NEB E. coli DNA ligase buffer in the presence of 50 mM KCl in a total volume of 10 microliters. The amounts of plant extract added to the reactions were 0.2 microliter, 0.04 microliter, 0.008 microliter, or 0.0016 microliter, incubated at 37° C. for 30 minutes, and run on a 0.8% TAE-agarose gel in the presence of Gelstar fluorescent DNA staining reagent. Little or no nicking activity was detected in uninfected plant extracts whereas extracts from plants infected with the GENEWARE®-Selaginella construct pLSB2225 showed appreciable amounts of activity against the plasmid DNA substrate.

After positive results were obtained in that assay, extracts of pLSB2225 infected plants were used in a GRAMMR reaction to test the ability of this enzyme to operate as a component of the mismatch resolution reaction in place of the GENEWARE®-produced CEL I enzyme.

### EXAMPLE 14

### Use of RES I in the GRAMMR Reaction

This example teaches that RES I can be used in place of native CEL I enzyme purified from celery in Genetic Reassortment By DNA Mismatch Resolution without any noticeable change in results.

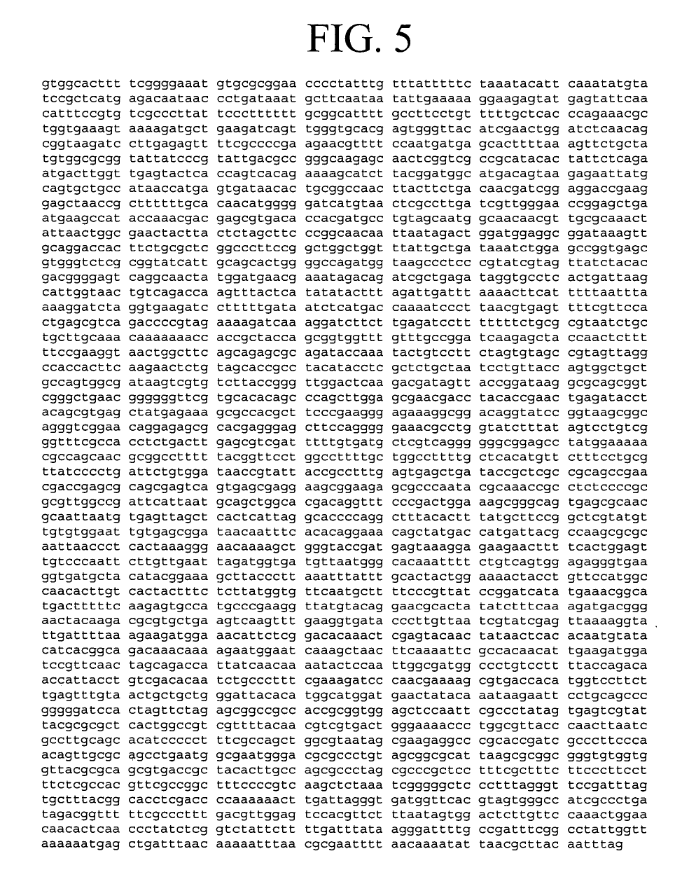

GRAMMR was performed between the wild-type Aequorea victoria GFP gene (Prasher, et al., Gene111(92)229) in a pBS derivative (Stratagene, La Jolla, Calif.) encoded by pBSWTGFP (SEQ ID NO:31) and a variant with mutations to increase fluorescence intensity in E. coli, and to alter the emission wavelength to blue light emission (Crameri, et al., Nat Biotechnol 14(96)315; Heim et al., PNAS91(94)12501; Yang, et al., J Biol Chem 273(98)8212). This variant gene, encoded by the plasmid pBSC3BFP, as shown in FIG. 5 (SEQ ID NO: 32), encodes a fluorescent protein that emits bright blue light when excited by longwave UV light.

The GRAMMR reactions were performed on GFP/c3BFP heteroduplexes in a circular, double-stranded plasmid DNA context. The circular, whole-plasmid heteroduplex DNA substrates were prepared by first linearizing pBSWTGFP (SEQ ID NO:31) and pBSC3BFP (FIG. 5, SEQ ID NO: 32) by digestion with Kpn I and NgoM IV, respectively, then purifying the digested DNA using DNA spin columns. Next, 200 nanograms of each of the two linearized plasmids were mixed and brought to 1×SSPE (180 nM NaCl, 10 mM NaH₂PO₄, 1 mM EDTA at pH 7.4) in a volume of 20 microliters. The mixture was then incubated at 95 degrees Celsius for 4 minutes, plunged into icewater where it remained for 10 minutes prior to incubation at 37 degrees Celsius. After 30 minutes, the annealed DNA sample was then transferred back to ice where it was held until use in GRAMMR reactions.

Two independent series of shuffling reactions were performed to compare CEL I with RES I in their abilities to facilitate sequence shuffling by GRAMMR. Each GRAMMR reaction contained 1 unit of T4 DNA polymerase, 2 units of E. coli DNA ligase, and 5 nanomoles of each dNTP in 1×NEB E. coli ligase buffer supplemented with KCl to 50 mM. Two separate enzyme dilution series were then performed. To each of two series of tubes containing aliquots of the above cocktail, one microliter aliquots of GENEWARE®-expressed CEL I or RES I extracts at dilutions of ⅓, 1/9, 1/27, 1/81, or 1/243 were added. An endonuclease-free control reaction was also prepared. To each of the reactions, one microliter aliquots containing 20 nanograms of the annealed DNA heteroduplex substrate were added and the reactions incubated at room temperature for one hour and on ice for 30 minutes prior to transformation into competent E. coli.

Green fluorescent protein (GFP) and blue fluorescent protein (BFP) could be visualized in the resulting colonies by long wave UV illumination. The parental wild-type GFP has dim green fluorescence, and the parental c3BFP gave bright blue fluorescence. In the genes encoding these fluorescent proteins, the sequences that determine the emission color and those that govern fluorescence intensity are at different positions from one another. It is expected that DNA shuffling would result in the “de-linking” of the sequences that determine the emission color from those that govern fluorescence intensity. As a consequence, the resultant progeny would be expected to exhibit reassortment of the functional properties of emission color and intensity. Therefore a measure of the extent of the DNA shuffling that had taken place in each reaction could be scored by examining the color and intensity of fluorescence from the bacterial colonies on the corresponding plates. In the zero-nuclease control, only dim green and bright blue colonies were observed. However, on plates with cells transformed with DNAs from the reactions containing either CEL I or RES I, some bright green as well as some dim blue colonies were observed, indicating that shuffling of DNA sequences had taken place. DNA sequence analysis confirmed that this was indeed the case and that on average, the recovery of shuffled clones was greater than 85% for both CEL I and RES I and that the number and distribution of information transfer events was similar for both enzymes. However, it appeared that the activity of RES I in this experiment was several-fold higher than that of CEL I, as indicated by the low transformation efficiency of reactions treated with the higher concentrations of the RES I preparation.

### EXAMPLE 15

### Molecular Breeding of Highly Divergent Tobamovirus 30K Genes in Viral Vectors Using Plasmid-on-Plasmid Genetic Reassortment By DNA Mismatch Resolution (POP GRAMMR)

Example 10 taught the reassortment of movement protein (MP) genes from several divergent strains of tobamovirus (approximately 75% identical; cloned into the pGENEWARE-MP-Avr-Pac vector) using GRAMMR. This example teaches the use of Plasmid-on-plasmid GRAMMR (POP GRAMMR) for reassorting even more highly divergent species.

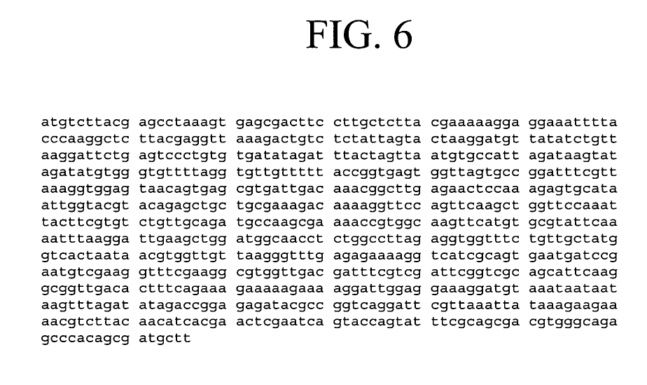

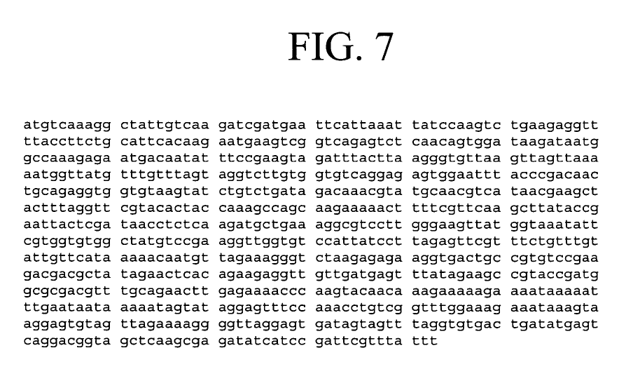

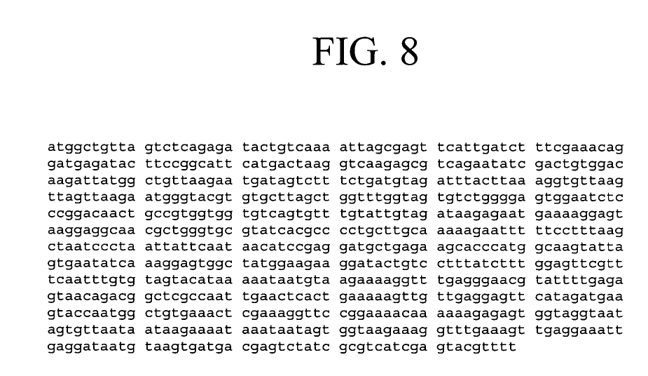

Starting parental MP genes from the tobamoviruses TMV-Cg (FIG. 6, SEQ ID NO: 18), TMV-Ob (FIG. 7, SEQ ID NO: 19), TMV-U2 (FIG. 8, SEQ ID NO: 20), TMV-U1 (SEQ ID NO: 9), and tomato mosaic virus (ToMV) (SEQ ID NO: 10) were used. The plasmid of pGENEWARE-ToMV MP was linearized by digestion with Sma I. The plasmids of pGENEWARE containing the MP genes from either TMV-Cg, TMV-Ob, TMV-U2, or TMV-U1 were digested with Stu I. The digested pGENEWARE-MP constructs were purified using DNA spin columns. The following heterduplex pairs were generated: pGENEWARE-Cg MP and pGENEWARE-ToMV MP, pGENEWARE-TMV-Ob MP and pGENEWARE-ToMV MP, pGENEWARE-TMV-U2 MP and pGENEWARE-ToMV MP, pGENEWARE-TMV-U1 MP and pGENEWARE- ToMV MP. The heteroduplexes of these MP gene sequences are approximately 47%, 58%, 62%, and 75% identical, respectively. Heteroduplex DNA was generated by mixing 200 nanograms of each of the two linearized plasmids in 1×SSPE (180 mM NaCl, 10 mM NaH₂PO₄, 1 mM EDTA, at pH 7.4) in a volume of 20 microliters. The mixture was incubated at 95 degrees Celsius for 4 minutes, plunged into ice water where it remained for 10 minutes prior to incubation at 37 degrees Celsius. After 30 minutes, the annealed DNA sample was then transferred back to ice where it was held until use in GRAMMR reactions.

Each 10 microliter GRAMMR reaction contained I unit of T4 DNA polymerase, 2 units of E. coli DNA ligase, and 0.5 mM of each dNTP in 1×NEB E. coli DNA ligase buffer supplemented with KCl to 50 mM. A one microliter aliquot of CEL I (diluted ⅓, 1/9, 1/27, 1/81, 1/243, or 1/729) was next added. An endonuclease-free control reaction was also prepared. To each of the reactions, a one microliter aliquot containing 20 nanograms of the annealed DNA heteroduplex substrate was added and the reactions were incubated at room temperature for one hour and on ice for 30 minutes prior to transformation into competent E. coli.

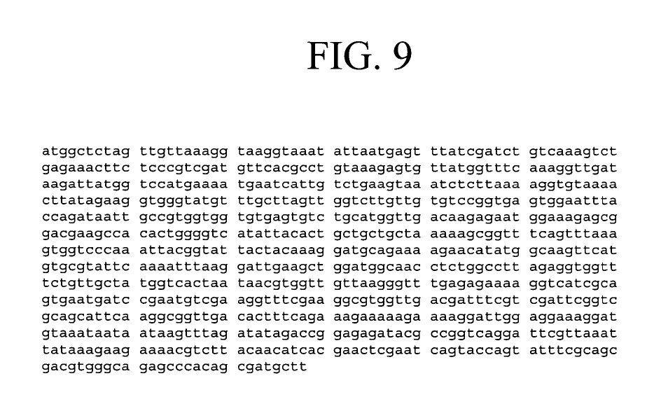

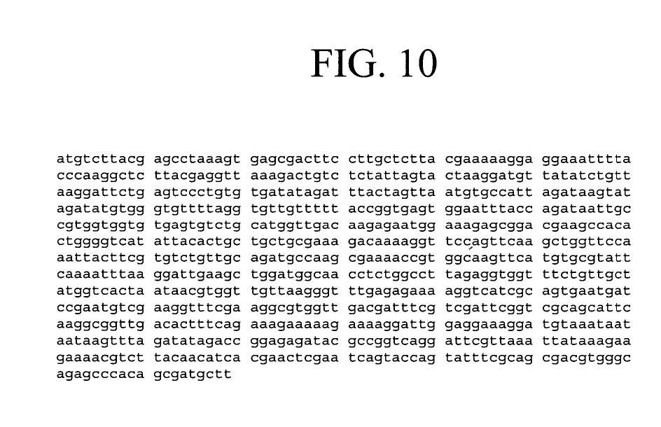

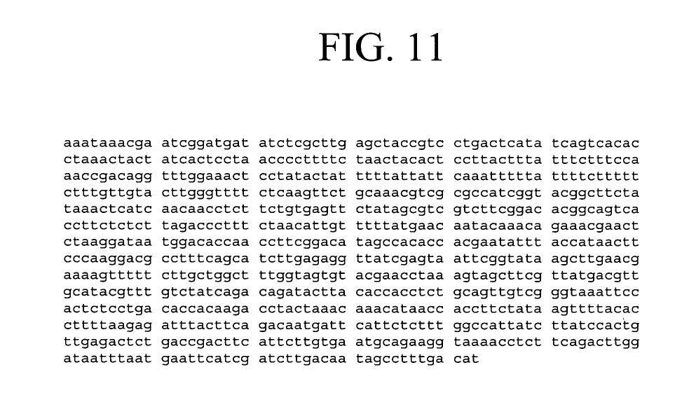

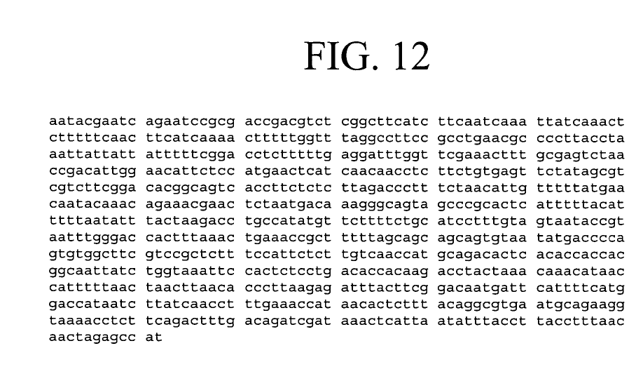

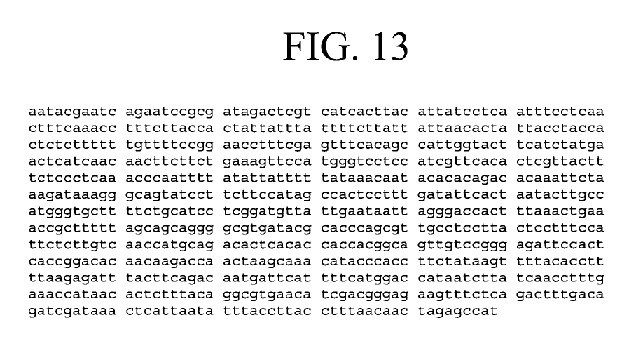

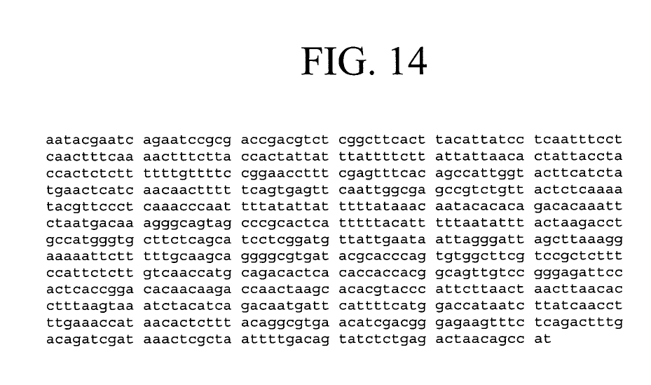

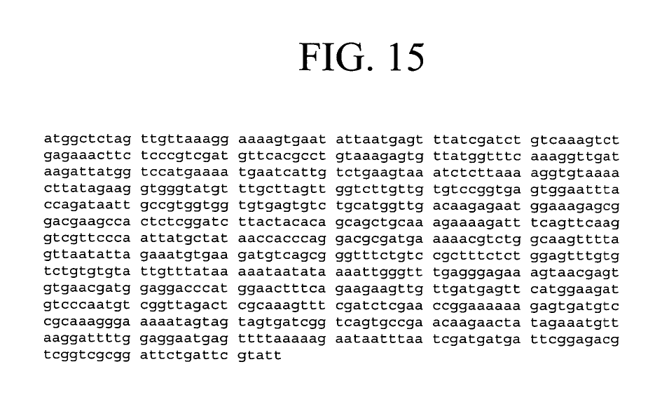

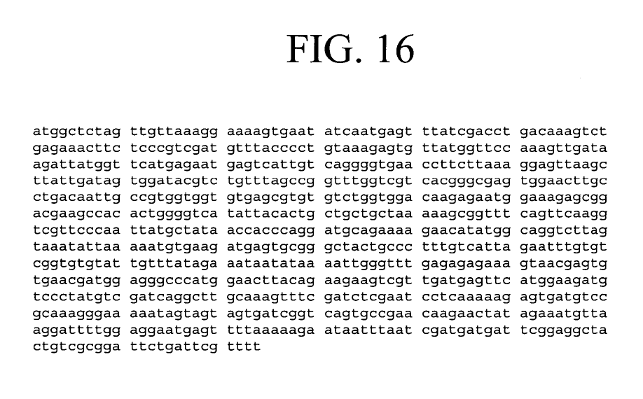

DNA sequence analysis was performed from both directions, and the sequence data showed that a significant number of clones derived from the GRAMMR-treated material were reassorted sequences containing information from both parental movement protein gene sequences. The DNA sequences of several exemplary out output pGENEWARE-MP clones from the GRAMMR reaction are shown as follows, TMV-Cg/ToMV clones, FIG. 9, SEQ ID NO: 21, and FIG. 10, SEQ ID NO: 22; TMV-Ob/ToMV clones, FIG. 11, SEQ ID NO: 23, and FIG. 12, SEQ ID NO: 24; TMV-U2/ToMV clones, FIG. 13, SEQ ID NO: 25, and FIG. 14, SEQ ID NO: 26; and TMV-U1/ToMV clones, FIG. 15, SEQ ID NO: 27, and FIG. 16, SEQ ID NO: 28.

### EXAMPLE 16

### GRAMMR On Linearized DNA Substrate Using Endonucleases That Cleave Within A Selectable Marker

This example teaches a GRAMMR reaction where DNA substrate molecules are linearized with restriction endonucleases that cleave within a selectable marker gene.

GRAMMR is performed between the wild-type Aequorea victoria GFP gene (Prasher, et al., Gene111(92)229) in a pBS derivative (Stratagene, La Jolla, Calif.) encoded by pBSWTGFP (SEQ ID NO:31) and a variant with mutations to increase fluorescence intensity in E. coli, and to alter the emission wavelength to blue light emission (Crameri, et al., Nat Biotechnol 14 (96) 315; Heim et al., PNAS91(94)12501; Yang, et al., J Biol Chem 273(98)8212). This variant gene, encoded by the plasmid pBSC3BFP (SEQ ID NO: 32), encodes a fluorescent protein that emits bright blue light when excited by longwave UV light.

The GRAMMR reactions are performed on GFP/c3BFP heteroduplexes in a circular, double-stranded plasmid DNA context. The circular, whole-plasmid heteroduplex DNA substrates are prepared by first linearizing pBSWTGFP (SEQ ID NO:31) and pBSC3BFP (SEQ ID NO: 32) by digestion with Ahd I and Bcg I, respectively, then purifying the digested DNA using DNA spin columns. Next, 200 nanograms of each of the two linearized plasmids are mixed and brought to 1×SSPE (180 nM NaCl, 10 mM NaH2PO4, 1 mM EDTA at pH 7.4) in a volume of 20 microliters. The mixture is then incubated at 95 degrees Celsius for 4 minutes, plunged into icewater where it remains for 10 minutes prior to incubation at 37 degrees Celsius. After 30 minutes, the annealed DNA sample is then transferred back to ice where it is held until use in GRAMMR reactions.

Two independent series of reassortment reactions are performed to compare CEL I with RES I in their abilities to facilitate sequence reassortment by GRAMMR. Each reaction is first treated for 10 minutes at room-temperature with 1 unit of T4 DNA polymerase in the presence of 5 nanomoles of each dNTP in 1×NEB E. coli ligase buffer supplemented with KCl to 50 mM. Subsequently, 2 units of E. coli DNA ligase are added. Two separate enzyme dilution series are then performed. To each of two series of tubes containing aliquots of the above cocktail, one microliter aliquots of GENEWARE®-expressed CEL I or RES I extracts at dilutions of ⅓, 1/9, 1/27, 1/81, or 1/243 are added. An endonuclease-free control reaction is also prepared. To each of the reactions, one microliter aliquots containing 20 nanograms of the annealed DNA heteroduplex substrate are added and the reactions incubated at room temperature for one hour and on ice for 30 minutes prior to transformation into competent E. coli.

Green fluorescent protein (GFP) and blue fluorescent protein (BFP) is visualized in the resulting colonies by long wave UV illumination. The parental wild-type GFP gives dim green fluorescence, and the parental c3BFP gives bright blue fluorescence. In the genes encoding these fluorescent proteins, the sequences that determine the emission color and those that govern fluorescence intensity are at different positions from one another.

It is expected that DNA reassortment would result in the “de-linking” of the sequences that determine the emission color from those that govern fluorescence intensity. As a consequence, the resultant progeny would be expected to exhibit reassortment of the functional properties of emission color and intensity. Therefore a measure of the extent of the DNA reassortment that had taken place in each reaction can be scored by examining the color and intensity of fluorescence from the bacterial colonies on the corresponding plates.

### EXAMPLE 17

### GRAMMR On Linearized DNA Substrate Using Endonucleases That Cleave Within A Selectable Marker

This example teaches a GRAMMR process where DNA substrate molecules are linearized with restriction endonucleases that cleave within a selectable marker gene.

GRAMMR reassortment is performed between the wild-type Aequorea victoria GFP gene (Prasher, et al., Gene111(92)229) in a pBS derivative (Stratagene, La Jolla, Calif.) encoded by pBSWTGFP (SEQ ID NO:03) and a variant with mutations to increase fluorescence intensity in E. coli, and to alter the emission wavelength to blue light emission (Crameri, et al., Nat Biotechnol 14 (96) 315; Heim et al., PNAS91(94)12501; Yang, et al., J Biol Chem 273(98)8212). This variant gene, encoded by the plasmid pBSC3BFP (SEQ ID NO: 17), encodes a fluorescent protein that emits bright blue light when excited by longwave UV light.

The GRAMMR reactions are performed on GFP/c3BFP heteroduplexes in a circular, double-stranded plasmid DNA context. The circular, whole-plasmid heteroduplex DNA substates are prepared by first linearizing pBSWTGFP (SEQ ID NO:03) and pBSC3BFP (SEQ ID NO: 17) by digestion with Ahd I and Bcg I, respectively, then purifying the digested DNA using DNA spin columns. Next, 200 nanograms of each of the two linearized plasmids are mixed and brought to 1×SSPE (180 nM NaCl, 10 mM NaH2PO4, 1 mM EDTA at pH 7.4) in a volume of 20 microliters. The mixture is then incubated at 95 degrees Celsius for 4 minutes, plunged into icewater where it remains for 10 minutes prior to incubation at 37 degrees Celsius. After 30 minutes, the annealed DNA sample is then transferred back to ice where it is held until use in GRAMMR reactions.

Two independent series of reassortment reactions are performed to compare CEL I with RES I in their abilities to facilitate sequence reassortment by GRAMMR. Each reaction is first treated for 10 minutes at room-temperature with 1 unit of T4 DNA polymerase in the presence of 5 nanomoles of each dNTP in 1×NEB E. coli ligase buffer supplemented with KCl to 50 mM. Subsequently, 2 units of E. coli DNA ligase are added. Two separate enzyme dilution series are then performed. To each of two series of tubes containing aliquots of the above cocktail, one microliter aliquots of GENEWARE-expressed CEL I or RES I extracts at dilutions of ⅓, 1/9, 1/27, 1/81, or 1/243 are added. An endonuclease-free control reaction is also prepared. To each of the reactions, one microliter aliquots containing 20 nanograms of the annealed DNA heteroduplex substrate are added and the reactions incubated at room temperature for one hour and on ice for 30 minutes prior to transformation into competent E. coli.

Green fluorescent protein (GFP) and blue fluorescent protein (BFP) are visualized in the resulting colonies by long wave UV illumination. The parental wild-type GFP gives dim green fluorescence, and the parental c3BFP gives bright blue fluorescence. In the genes encoding these fluorescent proteins, the sequences that determine the emission color and those that govern fluorescence intensity are at different positions from one another.

It is expected that DNA reassortment would result in the “de-linking” of the sequences that determine the emission color from those that govern fluorescence intensity. As a consequence, the resultant progeny would be expected to exhibit reassortment of the functional properties of emission color and intensity. Therefore a measure of the extent of the DNA reassortment that had taken place in each reaction can be scored by examining the color and intensity of fluorescence from the bacterial colonies on the corresponding plates.

### EXAMPLE 18

### Use of Other Nucleases in the GRAMMR Reaction

This example teaches that nucleases other than mismatch endonucleases can be used in Genetic Reassortment By DNA Mismatch Resolution.

GRAMMR shuffling was performed between the wild-type Aequorea victoria GFP gene (Prasher, et al., Gene111(92)229) in a pBS derivative (Stratagene, La Jolla, Calif.) encoded by pBSWTGFP (SEQ ID NO:03) and a variant with mutations to increase fluorescence intensity in E. coli, and to alter the emission wavelength to blue light emission (Crameri, et al., Nat Biotechnol 14(96)315; Heim et al., PNAS91(94)12501; Yang, et al., J Biol Chem 273(98)8212). This variant gene, encoded by the plasmid pBSC3BFP, as shown in FIG. 5 (SEQ ID NO: 17), encodes a fluorescent protein that emits bright blue light when excited by longwave UV light.

The GRAMMR reactions were performed on GFP/c3BFP heteroduplexes in a circular, double-stranded plasmid DNA context. The circular, whole-plasmid heteroduplex DNA substrates were prepared by first linearizing pBSWTGFP (SEQ ID NO:03) and pBSC3BFP (FIG. 5, SEQ ID NO: 17) by digestion with Kpn I and NgoM IV, respectively, then purifying the digested DNA using DNA spin columns. Next, 200 nanograms of each of the two linearized plasmids were mixed and brought to 1×SSPE (180 nM NaCl, 10 mM NaH₂PO₄, 1 mM EDTA at pH 7.4) in a volume of 20 microliters. The mixture was then incubated at 95 degrees Celsius for 4 minutes, plunged into icewater where it remained for 10 minutes prior to incubation at 37 degrees Celsius. After 30 minutes, the annealed DNA sample was then transferred back to ice where it was held until use in GRAMMR reactions.

A number of independent series of shuffling reactions were performed to determine whether non-mismatch-specific nucleases could facilitate sequence shuffling by GRAMMR. Each GRAMMR reaction contained 1 unit of T4 DNA polymerase, 2 units of E. coli DNA ligase, and 5 nanomoles of each dNTP in 1×NEB E. coli ligase buffer supplemented with KCl to 50 mM. Two separate enzyme dilution series were then performed. To each of six series of tubes containing aliquots of the above cocktail, one microliter aliquots of various nucleases, including Bal 31 (New England Biolabs), DNAseI (Ambion), mung bean nuclease (New England Biolabs), RQ1 DNAse (Promega), S1 nuclease (BRL), or phage T7 Endonuclease I (New England Biolabs), all adjusted to one unit per microliter by dilution in E. coli DNA ligase buffer, and at dilutions of ⅓, 1/9, 1/27, 1/81, or 1/243 were added. Another series of enzyme dilutions was prepared with TMV viral vector-expressed CEL I. An endonuclease-free control reaction was also prepared. To each of the reactions, one microliter aliquots containing 20 nanograms of the annealed DNA heteroduplex substrate were added and the reactions incubated at room temperature for one hour and on ice for 30 minutes prior to transformation into competent E. coli.

Green fluorescent protein (GFP) and blue fluorescent protein (BFP) could be visualized in the resulting colonies by long wave UV illumination. The parental wild-type GFP has dim green fluorescence, and the parental c3BFP gave bright blue fluorescence. In the genes encoding these fluorescent proteins, the sequences that determine the emission color and those that govern fluorescence intensity are at different positions from one another. It is expected that DNA shuffling would result in the “de-linking” of the sequences that determine the emission color from those that govern fluorescence intensity. As a consequence, a fraction of the resultant progeny would be expected to exhibit reassortment of the functional properties of emission color and intensity. Therefore a measure of the extent of the DNA shuffling that had taken place in each reaction could be scored by examining the color and intensity of fluorescence from the bacterial colonies on the corresponding plates.

In the zero-nuclease control, only dim green and bright blue colonies were observed. The same was observed in the reactions containing nuclease Bal 31, mung bean nuclease, S1 nuclease, and T7 endonuclease I. However, on plates with cells transformed with DNAs from the reactions containing CEL I, DNAseI, or RQ1 DNAse, some bright green as well as some dim blue colonies were observed, indicating that shuffling of DNA sequences had taken place. More bright green colonies were obtained on the plates corresponding to CEL I treatment than those representing RQ1 DNAse treatment, which in turn, had more than the number observed for the DNAseI treatment. The negative results obtained using T7 endonuclease I were subsequently discounted, however, as it was found in other experiments that the enzyme preparation that was used lacked detectable nuclease activity.

### EXAMPLE 19

### Comparison of RQ1, DNAse, and DNAse I Activity

Another set of experiments was performed to reconfirm some of the above results. Reactions were prepared as above in which RQ1 DNAse, DNAse I (diluted to one unit per microliter) and CEL I were diluted to ⅓, 1/9, 1/27, 1/81, 1/243, 1/729, or 1/2187, and one microliter aliquots of each were added to the reaction cocktail prior to addition of the heteroduplex DNA. An endonuclease-free control reaction was also prepared.

The results were similar to those obtained in the previous example. A total of ˜500 colonies were obtained for each enzyme series. A total of twenty bright green colonies were observed on the CEL I series, whereas six bright green colonies were found on the plates corresponding to RQ1 DNAse-treatment. No bright green colonies were observed on the DNAse I plates or on the no-nuclease control plates.

### EXAMPLE 18

### Alternative Method for DNA Shuffling by Using DNAase I and pol I

This experiment replicates those described by Moore et al., WO 02/24953 in which heteroduplex DNA is treated with a non-specific endonuclease (DNase I). Subsequently, heteroduplex DNA is contacted with a nick-translating DNA polymerase (Pol I) which nick-translates on the heteroduplex DNA to bring-about a form of DNA shuffling.

GFP and c3BFP genes were used in the experiment. Heteroduplexes between the GFP/c3BFP gene were generated in a circular, double-strnaded plasmid DNA context. The circular, whole-plasmid heteroduplex DNA substrates were prepared by first linearizing pBSWTGFP (SEQ ID NO:31) and pBSC3BFP (FIG. 5, SEQ ID NO: 32) by digestion with Kpn I and NgoM IV, respectively, then purifying the digested DNA using DNA spin columns. Next, 125 nanograms of each of the two linearized plasmids were mixed in a volume of 10 microliters and incubated at 95degrees Celsius for 4 minutes, plunged into ice water for 10 minutes. Subsequently, 1.1 ul of 10×SSPE (1800 mM NaCl, 100 mM NaH₂PO₄, 10 mM EDTA at pH 7.4) was added prior to incubation at 37 degrees Celsius After 30 minutes, the annealed DNA sample was then transferred back to ice. The sample was run out on a 2% low melt agarose gel and the nicked-circular heteroduplex band was gel isolated and purified using a DNA spin column.

The following reagents were mixed on ice: 5.4 microliters water; 1.0 microliters 10×NT buffer (0.5M Tris-HCl pH 7.5; 0.1M MgCl₂; 10 mM dithiothreitol [DTT]; 0.5 mg/mL BSA); 0.4 microliters Pol I (4 units), 1.8 microliters 2 mM dNTP, 0.4 microliters DNase I (0.18 units; diluted from 10 units/ul stock in 1×NT buffer in 50% glycerol); and one microliter heteroduplex DNA (20 ng). Control reactions in which lacked either or both DNase I or Pol I were also set up. All reactions were carried out at 14 degrees Celsius for 15 mins and stopped with 0.5 microliters 500 mM EDTA. One microliter of the reaction was transformed into competent E. coli.

DNA sequence analysis was performed from both directions. In reactions containing both DNase I and Pol I, results showed that 44% of the clones analyzed were chimeras of the two parent genes. Each clone contained only one crossover site. In addition, all these chimeras were made up of c3BFP sequences upstream of the crossover site and wild-type GFP sequences downstream of the crossover site. These marked polarity effects and exclusively single-crossover chimeras are consistent with what would be expected from a purely nick-translation based mechanism of DNA shuffling. In the control reaction lacking DNase I, 34% of the clones analyzed were chimeras of the two parent genes and also exhibited the same polarity effect as observed with the DNase I plus Pol I reaction. In the control reaction lacking Pol I, 17% of the clones analyzed were chimeras of the two parent genes. In the control reaction lacking both DNase I and Pol I, 10% of the clones analyzed were chimeras of the two parent genes.

### EXAMPLE 21

### Use of Varying Ratios of DNA Polymerases and DNA Ligase to Regulate the Granularity of the Genetic Reassortment by DNA Mismatch Resolution Reaction

This experiment teaches that the length of sites of information transfer (granularity) can be regulated by manipulating the concentrations of certain components of a GRAMMR reaction.

The longer the blocks of sequence information transferred, the coarser the granularity. The shorter the blocks of sequence information transferred, the finer the granularity.

The GFP and c3BFP genes were used in the experiment. Heteroduplexes between the GFP/c3BFP genes were generated in a circular, double-stranded plasmid DNA context as described in Example 14. Matrix experiments were performed in which the relative concentration of DNA polymerase and DNA ligase in the GRAMMR was varied. NEB E. coli DNA polymerase I (Pol I, which after proofreading, can nick-translate from sites on CEL I nicking) and NEB E. coli DNA ligase were used. These two enzymes were diluted from the stock concentration in 1× E. coli ligase buffer. The concentrations of Pol I used were 0.01, 0.1, 1.0, and 5.0 units/uL. The concentrations of E. coli DNA ligase used were 0.0, 0.02, 0.2, and 2.0 units/uL. In all, the matrix contained 16 individual reactions. Each reaction contained 0.5 mM of each dNTP, 1×NEB E. coli ligase buffer supplemented with KCl to 50 mM, one microliter of diluted E. coli DNA ligase, one microliter of diluted Pol I, one microliter of a GENEWARE®-expressed CEL I preparation (containing 27 ng protein), and 20 nanograms of the annealed DNA heteroduplex. The reactions were incubated at room temperature for one hour before direct transformation into competent E. coli.

DNA sequence analysis was performed from both directions on a number of randomly-selected clones, and the sequence data showed varying degrees of granularity and crossover frequency among progeny clones depending on the relative concentration of Pol I to DNA ligase used. For instance, in reactions in which no DNA ligase and 1.0 units of Pol I was used, progeny clones showed a larger granularity with only one crossover between the parental clones. In reactions in which 0.2 units of DNA ligase and 0.1 units of Pol I was used, the granularity was finer with an average of approximately three crossovers between parental clones. In reactions in which 2.0 units of DNA ligase and 0.1 units of Pol 1 was used, the granularity was relatively much finer with an average of approximately seven crossovers between parental clones.

From this experiment, a trend emerged where the higher the ligase: Pol I ratio, the finer the granularity. When the concentration of ligase is low in relation to Pol I, it is likely that the Pol I enzyme can nick-translate for longer distances before the nick becomes sealed by the ligase. However, as the concentration of ligase is increased, the potential for nick-sealing is increased, which will tend to terminate nick-translation events earlier, thus shortening the average length of the sites of information transfer.

### EXAMPLE 22

### Plasmid-on-Plasmid Zonal Mutagenesis using Genetic Reassortment by DNA Mismatch Resolution (POP zmGRAMMR) of GFP Plasmids

This example teaches that random or semi-random mutations can be incorporated at and in the immediate vicinity of mismatched residues by performing GRAMMR in the presence of nucleotide analogs that have multi base-pairing potential. The end result is a population of shuffled genes with random mutations concentrated in regions of heterogeneity between the starting genes.

Unlike conventional GRAMMR methods, zonal mutagenesis GRAMMR requires only one nucleotide pair mismatch in the heteroduplex. Instead of resolving diversity between the two polynucleic acids in the heteroduplex, one is increasing diversity. One of the polynucleotides need not be full length and may be an oligonucleotide sufficiently long to hybridize and still have one base mismatched. It is partially complementary to the desired polynucleotide strand. In this manner, one can direct mutagenesis to a particular zone using a synthetic oligo or polynucleotide without ever having full length parent strands with a mismatch at or near the zone.

The mutagenesis zone on each strand forming the heteroduplex includes the mismatched base pair and a region within 1 to about 50 nucleotides upstream and downstream on both strands. More preferably, the mutagenesis zone includes the mismatched base pair and 1 to about 10 nucleotides on either side of the mismatch.

The nucleotide analogue may be any one or a combination of plural nucleotide analogues which will induce a change in base in the same or a complementary strand immediately or after replication of the polynucleotide strand incorporating the nucleotide analogue or a complementary strand. The nucleotide analogue may also induce an insertion or deletion of one or more nucleotides on either strand. A large number of nucleotide analogues are known per se.

Heteroduplexes between the GFP/c3BFP genes were generated in a circular, double-stranded plasmid DNA context as described in Example 14. Several zmGRAMMR reactions were set up in which the ratio of ligase to polymerase and analog nucleotide to dNTP ratios varied. The zmGRAMMR reactions contained the following: 0.1 unit of Pol I DNA polymerase; 2 or 10 units of E. coli DNA ligase; 0 or 0.5 mM of 2′-deoxy-P-nucleoside-5′-triphosphate (dPTP); 0 or 0.5 mM 8-oxo-2′ -deoxyguanosine-5′-triphosphate (8-oxo-dGTP); 0, 5, 25, 50, or 500 nM of each dNTP; and 1×NEB E. coli ligase buffer supplemented with KCl to 50 mM. Reactions were also set up in which 1 unit of T4 DNA polymerase or 5 units of Klenow polymerase was used in lieu of Pol I. A one microliter aliquot of a GENEWARE®-expressed RES I preparation containing 2 ng protein was then added. An endonuclease-free control reaction was also prepared. Finally, 20 nanograms of the annealed DNA heteroduplex substrate was added and the complete reaction was incubated at 25° C. for one hour. The zmGRAMMR treated heteroduplex was then column purified and transformed into competent E. coli.

The resulting colonies were examined under UV illumination. ZmGRAMMR reactions with high concentrations of nucleotide analog relative to dNTPs gave rise to a high proportion of non-fluorescent colonies, whereas colonies resulting from control reactions performed in the absence of analog showed few, if any non-fluorescent colonies. Reactions containing Klenow polymerase gave rise to very few colonies, all of which were non-fluorescent. DNA sequence analysis performed on a number of randomly-picked clones showed that a significant number of clones derived from nucleotide analog-containing GRAMMR reactions contained mutations (i.e. sequences unrelated to both parents) focused at or very near sites of mismatch between the GFP/c3BFP genes. Reactions containing a higher ratio of nucleotide-analog to dNTP yielded a higher percentage of clones containing mutations. ZmGRAMMR reactions using Klenow were largely unsuccessful, as few colonies were recovered, even for control reactions with no analogs. Clones derived from zmGRAMMR reactions using T4 DNA polymerase showed mutations that were more focused to sites of mismatch than those from the Pol I containing reactions. This result was as expected, since T4 DNA polymerase does not nick-translate, and thus, is expected in incorporate analogs only at or very near the site of the excised mismatch.

As the base analogs are incorporated during the GRAMMR reaction, these mutations serve to mark the tract of the polymerase during the course of the reaction. By varying the ratios of ligase to Pol I as taught in example 18, the width of those mutated tracts can also be manipulated.

Deposits with The American Type Culture Collection (ATCC)

Three deposits have been made in the American Type Culture Collection (ATCC), 10801 University Blvd., Manassas, Va. A deposit has been made of a plasmid DNA construct containing a derivative of tobacco mosaic virus and cDNA of the CEL I mismatch-endonuclease gene from celery, tagged with 6HIS. The construct is internally designated P1177MP4-CEL I 6HIS, and has been assigned ATCC Number PTA-3927. A deposit has been made of a plasmid DNA construct containing a derivative of tobacco mosaic virus and cDNA of the CEL I mismatch-endonuclease gene from celery. The construct is internally designated P1177MP4-CEL I Avr, and has been assigned ATCC Number PTA-3926. A deposit has been made of a plasmid DNA construct containing a derivative of tobacco mosaic virus and a cDNA insert encoding a 34kDa protein from Selaginella lepidophylla. The cDNA insert is referred to as RES I-6HIS. RES I is a mismatch endonuclease gene. The construct is internally designated pLSB-2225, and has been assigned ATCC Number PTA-4562.

These deposits were made in accordance with the terms and provisions of the Budapest Treaty relating to deposit of microorganisms and was made for a term of at least thirty (30) years and at least five (05) years after the most recent request for the furnishing of a sample of the deposit is received by the depository, or for the effective term of a patent to issue from this application or a subsequent application citing any of these deposits, whichever is longer. Each deposit will be replaced if it becomes non-viable during that period.

It should be noted that applicant's designations for each of the clones were shortened in the deposit to the aforementioned deposit with the American Type Culture Collection, that is, p1177MP4-CEL I Avr-B3 is referred to as p 1177MP4-CEL I Avr; and p1177MP4-CEL I 6His-A9 is referred to as p1177MP4-CEL I 6His. The clone p1177MP4-CEL I Avr (SEQ ID NO:01) contained the CEL I open reading frame extending from nucleotide 5765 to 6655 (SEQ ID NO:03); and the clone p1177MP4-CEL I 6His-A9 (SEQ ID NO:02) contained the CEL I open reading frame extending from nucleotide 5765-6679 (SEQ ID NO:04).

We claim:

1. A method for preparing a variant polynucleotide having a different nucleotide sequence from at least two parent polynucleotides comprising;
preparing at least one heteroduplex between at least said two parent polynucleotides,
cleaving at least one polynucleotide strand in said heteroduplex at a mismatch site to form a cleavage site,
replacing at least one nucleotide on at least one polynucleotide strand at or near the cleavage site,
wherein at least one of said polynucleotide strands has a different nucleotide sequence from either of said at least two parent polynucleotides.

2. The method of claim 1 wherein the polynucleotides forming a heteroduplex are circular.

3. The method of claim 2 wherein said preparing is performed in vitro and said cleaving and said replacing are performed in vivo.

4. The method of claim 1 herein the heteroduplex polynucleotide sequence contains a gene.

5. The method of claim 4 wherein the heteroduplex polynucleotide sequence is a genome.

6. The method of claim 4 wherein the heteroduplex polynucleotide sequence is a region within a polynucleotide molecule of greater than about 100 bp.

7. The method of claim 1 wherein at least three parental polynucleotides are used.

8. The method of claim 1 wherein a plurality of variant polynucleotides are formed.

9. A method for obtaining a polynucleotide sequence encoding a desired functional property comprising;
preparing at least one heteroduplex from at least two parent polynucleotides;
cleaving at least one polynucleotide strand in said heteroduplex at a mismatch site to form a cleavage site,
replacing at least one nucleotide on at least one polynucleotide strand at or near the cleavage site, wherein a plurality of variant polynucleotide strands are formed which have different nucleotide sequences from either of said at least two parent polynucleotides, and
screening or selecting a population of variants for the desired functional property.

10. The method of claim 9 further comprising;
forming a second heteroduplex containing at least one variant polynucleotide strand,
cleaving at least one polynucleotide strand in said second heteroduplex at a mismatch site to form a cleavage site,
replacing at least one nucleotide on at least one polynucleotide strand at or near the cleavage site, wherein a plurality of variant polynucleotide strands are formed which have different nucleotide sequences from either parent polynucleotides present in said second heteroduplex, and
screening or selecting a population of variants for the desired functional property.

11. The method of claim 9 further comprising converting said variant polynucleotide to RNA before said screening or selecting.

12. The method of claim 9 further comprising translating said variant polynucleotide or an RNA transcribed therefrom to produce a polypeptide and wherein said screening or selecting is performed on said polypeptide.

13. A method of identifying a reasserted DNA molecule with a desired functional property, comprising:
a) providing at least one single-stranded modified polynucleotide, which single-stranded modified polynucleotide, or a complementary strand thereto has or encodes the desired functional property, wherein said modified polynucleotide is capable of hybridization but not amplification;
b) providing one or a plurality of non-identical single-stranded DNA molecules capable of hybridizing to the single-stranded modified polynucleotide, wherein said DNA molecules have or encode at least one additional variant;
contacting the single-stranded modified polynucleotide with at least one single-stranded DNA molecule of step (b), thereby producing an annealed DNA molecule;
incubating the annealed DNA molecule with a mismatch endonuclease, proofreading enzyme and a ligase, thereby producing a recombined DNA strand annealed to the single stranded modified polynucleotide; and
screening or selecting the population of reasserted DNA molecules to identify those that encode the desired functional property, thereby identifying one or more DNA molecules(s) that encode a polypeptide with the desired functional property.

14. The method of claim 13 further comprising amplifying the reasserted DNA strand under conditions wherein the modified polynucleotide is not amplified, thereby producing a population of reassorted DNA molecules before said screening or selecting.

15. The method of claim 13 wherein said modified polynucleotide contains uracil.

16. A method of identifying a recombined DNA molecule with a desired functional property, comprising:
forming an annealed double stranded molecule between at least
a. at least one single-stranded modified polynucleotide, which single-stranded modified polynucleotide, or a complementary strand thereto, wherein the modified polynucleotide is capable of hybridization but not amplification, and
b. at least one non-identical single-stranded DNA capable of hybridizing to the single-stranded modified polynucleotide, wherein said DNA molecules have or encode at least one additional variant;
cleaving at least the DNA strand of the annealed double stranded molecule to form a cleavage site, and
replacing a nucleotide at or near the cleavage site, thereby producing a recombined DNA strand annealed to the modified polynucleotide; wherein said recombined DNA strand has a different nucleotide sequence from either polynucleotide of said annealed double stranded molecule, and
screening or selecting the population of recombined DNA molecules to identify those that have or encode the desired functional property, thereby identifying one or more DNA molecules(s) that have or encode the desired functional property.

17. The method of claim 16 further comprising amplifying the recombined DNA strand under conditions wherein the modified DNA molecule is not amplified, thereby producing a population of recombined DNA molecules, before said screening or selecting.

18. The method of claim 16 wherein the cleaving is at a mismatch site.

19. The method of claim 16 wherein said modified polynucleotide contains uracil.

20. A composition for a method of making sequence variants in vitro from at least one heteroduplex polynucleotide where said heteroduplex has at least two non-complementary nucleotide base pairs, said composition comprises effective amounts of an agent or agents with exonuclease activity, polymerase activity and mismatch strand cleavage activity.

21. The composition of claim 20 further comprising a ligase activity.

22. The composition for a method of making sequence variants in vitro from at least one heteroduplex polynucleotide where said heteroduplex has at least two non-complementary nucleotide base pairs, said composition comprises effective amounts of an agent or agents with 3′ to 5′ proofreading exonuclease activity, polymerase activity and strand cleavage activity.

23. The composition of claim 22 further comprising a ligase activity.

24. A kit used for making sequence variants in vitro from at least one heteroduplex polynucleotide where said heteroduplex has at least two non-complementary nucleotide base pairs, wherein said kit comprises a plurality of vessels containing an effective amount of an agent or agents with exonuclease activity, polymerase activity and strand cleavage activity.

25. The kit of claim 24 further comprising an agent or agents having a ligase activity.

26. A nicked circular heteroduplex which has at least two non-complementary nucleotide base pairs and a nick located at at least one of the two non-complementary nucleotide base pairs.

27. The nicked circular heteroduplex of claim 26 wherein a nick is located at least two of said at least two non-complementary nucleotide base pairs.

28. A method for altering the length of polynucleic acid sequences resolved within a heteroduplex comprising;
a. preparing at least one heteroduplex polynucleotide;
b. combining said heteroduplex polynucleotide with an effective amount of an agent or agents with at least exonuclease activity, polymerase activity, ligase activity and strand cleavage activity; and
c. allowing sufficient time for the percentage of complementarity between strands of said heteroduplex polynucleotide to increase,
wherein the ratio of polymerase activity to ligase activity is altered to obtain a desired length of resolution between the polynucleotide strands of the heteroduplex.

29. The method of claim 28 wherein said strand cleavage activity is a mismatch strand cleaving activity.

30. A method of generating mutations in a zone around a particular nucleotide within a desired polynucleotide comprising;
a. preparing at least one partially complementary polynucleotide having at least one non-complementary nucleotide within the zone around the desired polynucleotide,
b. forming at least one heteroduplex between said desired polynucleotide and said partially complementary polynucleotide;
c. combining said heteroduplex with at least one nucleotide analogue, effective amount of an agent or agents with exonuclease activity, polymerase activity, and mismatch strand cleavage activity; and
d. allowing sufficient time for the nucleotide analogue to be incorporated into either said desired polynucleotide, said partially complementary polynucleotide or both.

31. The method according to claim 30 further comprising having a ligase activity.

32. A non-naturally occurring variant polynucleotide produced by the process of claim 1, 13, 18, 20 or 29.

33. The non naturally occurring variant polynucleotide of claim 32 having a desired functional property

34. A non-naturally occurring variant polypeptide produced by the process of claims 13, 18 or 20 having a desired functional property.

35. The non-naturally occurring polypeptide according to claim 34 wherein the desired functional property is different from a corresponding desired functional property of a polypeptide encoded by a parent polynucleotide.

36. A non-naturally occurring mutated polynucleotide produced by the process of claim 30.

37. The non-naturally occurring mutated polynucleotide according to claim 36 wherein the mutation is present at or within 30 nucleotides of a mismatch in the heteroduplex.

38. A non-naturally occurring variant polynucleotide comprising at least two parts, a first nucleotide sequence and a second nucleotide sequence,
wherein the first nucleotide sequence is identical to or complementary to part of a first naturally occurring polynucleotide, and not identical to and not complementary to a corresponding part of a second naturally occurring polynucleotide,
wherein the second nucleotide sequence is identical to or complementary to part of a second naturally occurring polynucleotide, and not identical to and not complementary to a corresponding part of a first naturally occurring polynucleotide.

39. The non-naturally occurring variant polynucleotide of claim 38 further comprising a third nucleotide sequence wherein the third nucleotide sequence is identical to or complementary to part of a third naturally occurring polynucleotide and not identical to and not complementary to a corresponding part of the first naturally occurring sequence or the second naturally occurring sequence.

40. A non-naturally occurring variant polypeptide comprising at least two parts, a first peptide sequence and a second peptide sequence,
wherein the first peptide sequence is identical to or complementary to part of a first naturally occurring polypeptide, and not identical to and not complementary to a corresponding part of a second naturally occurring polypeptide,
wherein the second peptide sequence is identical to or complementary to part of a second naturally occurring polypeptide, and not identical to and not complementary to a corresponding part of a first naturally occurring polypeptide.

41. The non-naturally occurring variant polypeptide of claim 40 further comprising a third peptide sequence wherein the third peptide sequence is identical to or complementary to part of a third naturally occurring polypeptide and not identical to and not complementary to a corresponding part of the first naturally occurring sequence or the second naturally occurring sequence.
